SEQUENCE LISTING (101 sequences)

SEQ ID NO: 1 - protein, 192 aa, SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 2 - protein, 254 aa, SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 3 - protein, 62 aa, SARS-CoV-2
ATVCGPKKSTNLVKNKCVNFNFNGLTGTGVLTESNKKFLPFQQFGRDIADTTDAVRDPQTLE

SEQ ID NO: 4 - protein, 192 aa, mutant SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAP

SEQ ID NO: 5 - protein, 192 aa, mutant SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAP

SEQ ID NO: 6 - protein, 192 aa, mutant SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 7 - protein, 192 aa, Artificial Sequence
  SARS-CoV-2 RBD mutant
PNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAP

SEQ ID NO: 8 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 413 aa, MERS
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSVSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 10 - protein, 422 aa, SARS
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 11 - protein, 178 aa, SARS
PSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCG

SEQ ID NO: 12 - protein, 193 aa, MERS
PQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCP

SEQ ID NO: 13 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 563 aa, Artificial Sequence
  Construct 1
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPHHHHHH

SEQ ID NO: 15 - protein, 400 aa, Artificial Sequence
  Construct 2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGGGGSGGGGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPHHHHHH

SEQ ID NO: 16 - protein, 602 aa, Artificial Sequence
  Construct 3
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGGGGSGGGGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGGGGSGGGGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPHHHHHH

SEQ ID NO: 17 - DNA, 1688 bp, Artificial Sequence
  Construct 1
aatatcacaaacctgtgtccttttggggaggtgtttaacgcaacacgctttgcaagcgtgtacgcatggaatcgcaagagaatttccaactgcgttgccgactacagcgtgctgtacaattccgcttctttttccacatttaaatgttacggggtcagccccaccaaactgaatgacctctgtttcacaaatgtgtatgccgatagctttgttatccgcggcgatgaggttcggcagatcgctcccgggcagacaggaaaaatcgccgactacaattacaagctccccgacgatttcaccggttgcgtcattgcctggaactctaacaacctggattctaaggttggaggaaactacaattatctgtacagactcttccggaaaagcaacctcaaaccttttgagcgggatatctccaccgaaatctatcaggccgggagcactccctgcaatggtgtggaaggttttaactgctacttccctctgcagagctatgggttccaacctaccaacggcgtcggctaccagccttaccgggtggtcgttctgagctttgagctgctgcacgcacctggcggctggtcctccaagccaagaaaaggcatgggaacaaatctgtctgtgcccaaccctctcggtttcttccctgatcaccaactggatcccgcctttggcgcaaacagcaataatccagattgggactttaatccaatcaaagatcattggccagccgccaaccaggtcggtgtgggcgctttcggacccggcctcactccccctcatggcggcatcctcggctggagccctcaggcccaaggcatcctgacaactgtgtccaccattcccccaccagccagcacaaaccggcaatccggcagacagcctacccctatctctcccccactcagggattcccacccacaggccatgcaatggaatagcacagcttttcaccaggcactgcaggacccccgcgttagaggactgtatttccctgccggaggtagcagcagcggcactgtgaaccccgcccccaatattgcatcccatatctcttccatctctgccagaacaggagatcctgttaccaacccaaacatcactaacctgtgtccatttggggaggtgttcaacgcaactaggtttgcttccgtgtatgcttggaacagaaagaggatttctaattgtgtggctgactattctgtgctctacaatagcgcctccttcagcacattcaaatgctacggtgttagccccacaaagctgaatgacctctgttttactaacgtgtacgccgactctttcgttatcagaggcgatgaggtccgccagattgcccccgggcagacaggcaagatcgcagattataactacaagctcccagacgactttaccggctgcgtcattgcttggaattccaataacctggattccaaggttggcgggaactacaactatctctatagactgttcaggaaatctaacctgaagccttttgaaagggacatttccaccgaaatctatcaggctggtagcactccctgcaatggcgtcgaagggttcaactgttatttcccactgcagagctatggttttcagcctacaaacggcgtgggatatcaaccttaccgcgtggtggtcctgagctttgaactgctgcatgccccacatcaccaccaccatcactg

SEQ ID NO: 18 - DNA, 1805 bp, Artificial Sequence
  Construct 3
aatatcacaaacctgtgtccttttggggaggtgtttaacgcaacacgctttgcaagcgtgtacgcatggaatcgcaagagaatttccaactgcgttgccgactacagcgtgctgtacaattccgcttctttttccacatttaaatgttacggggtcagccccaccaaactgaatgacctctgtttcacaaatgtgtatgccgatagctttgttatccgcggcgatgaggttcggcagatcgctcccgggcagacaggaaaaatcgccgactacaattacaagctccccgacgatttcaccggttgcgtcattgcctggaactctaacaacctggattctaaggttggaggaaactacaattatctgtacagactcttccggaaaagcaacctcaaaccttttgagcgggatatctccaccgaaatctatcaggccgggagcactccctgcaatggtgtggaaggttttaactgctacttccctctgcagagctatgggttccaacctaccaacggcgtcggctaccagccttaccgggtggtcgttctgagctttgagctgctgcacgcacctggcggcggaggatccggaggaggaggctctcctaatattaccaacctgtgccccttcggagaggtttttaacgccacccgcttcgctagcgtgtatgcatggaataggaaaaggatttctaattgcgtggctgactactccgtgctctataattccgcctccttttctacctttaaatgctatggggtgtccccaaccaagctcaatgatctgtgctttaccaatgtgtatgctgattccttcgttatcagaggcgatgaggtccggcagatcgcccccgggcaaacaggcaagatcgctgactacaattataaactgccagatgacttcactggttgtgtgattgcctggaattctaataacctcgacagcaaggtcgggggcaattacaactatctgtatcggctgttcaggaaatctaatctcaagcccttcgaacgggatatttctactgagatctaccaggccggtagcactccatgtaacggtgtggaaggtttcaactgttacttcccactgcagagctacggcttccaaccaaccaacggggtgggctaccaaccctacagagtcgtggtgctgtctttcgagctgctgcatgcacctggcggcggaggatccggcggaggaggaagcccaaacatcactaacctgtgtccatttggggaggtgttcaacgcaactaggtttgcttccgtgtatgcttggaacagaaagaggatttctaattgtgtggctgactattctgtgctctacaatagcgcctccttcagcacattcaaatgctacggtgttagccccacaaagctgaatgacctctgttttactaacgtgtacgccgactctttcgttatcagaggcgatgaggtccgccagattgcccccgggcagacaggcaagatcgcagattataactacaagctcccagacgactttaccggctgcgtcattgcttggaattccaataacctggattccaaggttggcgggaactacaactatctctatagactgttcaggaaatctaacctgaagccttttgaaagggacatttccaccgaaatctatcaggctggtagcactccctgcaatggcgtcgaagggttcaactgttatttcccactgcagagctatggttttcagcctacaaacggcgtgggatatcaaccttaccgcgtggtggtcctgagctttgaactgctgcatgccccacatcaccaccaccatcactg

SEQ ID NO: 19 - protein, 173 aa, HBV
GGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTN

SEQ ID NO: 20 - protein, 173 aa, HBV
GGWSSKPRKGMGTNLSVPNPLGFFPDHQLDPAFKANSDNPDWDLNPHKDNWPDSNKVGVGAFGLGFTPPHGGLLGWSPQAQGILTTVPAAPPPASTNRQVARPPTPLSPPLRDTHPQAMQWNSTTFHQTLQDPRVRALYFPAGGSSSGTVNPVQNTASSISSILSKTGDPVPN

SEQ ID NO: 21 - protein, 173 aa, HBV
GGYSSKPRKGMGTNLSVPNPLGFLPDHQLDPAFGANSNNPDWDFNPNKDPWPEAWQVGVGAFGPGFTPPHGGLLGWSPQAQGILTPVPAAPPPASTNRQSGRQPTPISPPLRDSHPQAMQWNSTTFHQALLDPRVRGLYFPAGGSSSGTTNPVPTTASPISSIFSRTGDPAPN

SEQ ID NO: 22 - protein, 162 aa, HBV
GQNLSTSNPLGFFPDHQLDPAFRANTANPDWDFNPNKDTWPDANKVGAGAFGLGFTPPHGGLLGWSPQAQGILQTLPTNPPPASTNRQSGRQPTPLSPPLRNTHPQAMQWNSTTFHQTLQDPRVRGLYFPAGGSSSGTVNPVPTTVSHISSIFSRIGDPALN

SEQ ID NO: 23 - protein, 172 aa, HBV
GLSWTVPLEWGKNHSTTNPLGFFPDHQLDPAFRANTRNPDWDHNPNKDHWTAANKVGVGAFGPGFTPPHGGLLGWSPQAQGMLKTLPADPPPASTNRQSGRQPTPITPPLRDTHPQAMQWNSTTFHQALQDPRVRGLYFPAGGSSSGTVNPVPTTASLISSIFSRIGDPAPN

SEQ ID NO: 24 - protein, 173 aa, HBV
GAPLSTTRRGMGQNLSVPNPLGFLPDHQLDPLFRANSSSPDWDFNKSKDNWPMANKVGVGGYGPGFTPPHGGLLGWSPQAQGVLTTLPADPPPASTNRRSGRKPTPVSPPLRDTHPQAMQWNSTQFHQALLDPRVRALYFPAGGSSSETQNPAPTIASLTSSIFLKTGGPAMN

SEQ ID NO: 25 - protein, 172 aa, HBV
GLSWTVPLEWGKNLSTSNPLGFLPDHQLDPAFRANTNNPDWDFNPKKDPWPEANKVGVGAYGPGFTPPHGGLLGWSPQSQGTLTTLPADPPPASTNRQSGRQPTPISPPLRDSHPQAMQWNSTAFHQALQNPKVRGLYFPAGGSSSGIVNPVPTIASHISSIFSRIGDPAPN

SEQ ID NO: 26 - protein, 173 aa, HBV
GAPLSTARRGMGQNLSVPNPLGFFPDHQLDPLFRANSSSPDWDFNTNKDNWPMANKVGVGGFGPGFTPPHGGLLGWSPQAQGILTTSPPDPPPASTNRRSGRKPTPVSPPLRDTHPQAMQWNSTQFHQALLDPRVRGLYFPAGGSSSETQNPAPTIASLTSSIFSKTGDPAMN

SEQ ID NO: 27 - protein, 337 aa, Artificial Sequence
  FLAG::RBD::GPI
MNPLLILTFVAAALADYKDHDGDYKDHDIDYKDDDDKGGGGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLESLAVSTISSFSPPGYQVSFCLVMVLLFAVDTGLYFSVKTNI

SEQ ID NO: 28 - DNA, 1035 bp, Artificial Sequence
  FLAG::RBD::GPI
aagcttctcgagatgaatccactcctgatcctgaccttcgttgccgccgccctggcagattacaaagatcacgatggggactataaggatcatgatattgattacaaggacgacgacgataagggaggagggggatctccaaatattacaaatctgtgccctttcggagaggtgtttaatgcaactaggtttgcttctgtgtatgcatggaaccggaaacggattagcaattgcgtggccgattatagcgttctgtacaactctgcctcttttagcactttcaaatgctatggcgtcagccctaccaaactgaacgatctctgttttacaaacgtgtacgcagactcttttgtgatcagaggagacgaggtccgccagattgccccagggcagacagggaagattgctgactataactacaagctccccgatgactttacaggctgtgtgatcgcttggaattctaataacctcgactccaaggttggcgggaactacaattatctctatcggctgttcaggaaatccaatctgaagccatttgaaagggatatttctaccgagatctaccaggctggatccactccttgcaacggcgtcgaaggctttaactgttacttccccctgcaaagctatggctttcagcctaccaatggcgttggatatcagccctaccgcgtcgtcgtgctgtcttttgagctgctgcacgccccagccactgtttgtgggcccaagaaatccaccaacctggtgaagaacaaatgcgtcaacttcaattttaacggcctgaccggaactggcgtgctcactgagagcaacaagaaattcctgcccttccaacaattcggcagagacatcgcagacaccacagacgctgttagagacccccagaccctggaatccctcgccgtcagcacaatcagctccttcagccctcctgggtaccaggtgtccttctgtctcgtgatggtgctgctgttcgctgtggatacagggctctacttctccgtgaagaccaatatctaaagcggccgc

SEQ ID NO: 29 - DNA, 9633 bp, Artificial Sequence
  pMD-MLVogp
agcttggcccattgcatacgttgtatccatatcataatatgtacatttatattggctcatgtccaacattaccgccatgttgacattgattattgactagttattaatagtaatcaattacggggtcattagttcatagcccatattggagttccgcgttacataacttacggtaaatggcccgcctggctgaccgcccaacgacccccgccccttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttgtggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccctaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgaagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatccgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatccccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 30 - protein, 805 aa, Homo sapiens [sequence omitted]

SEQ ID NO: 31 - protein, 5 aa, Artificial Sequence
  Linker
GGGGS

SEQ ID NO: 32 - protein, 6 aa, Artificial Sequence
  Linker
GGGGSG

SEQ ID NO: 33 - protein, 7 aa, Artificial Sequence
  Linker
GGGGSGG

SEQ ID NO: 34 - protein, 8 aa, Artificial Sequence
  Linker
GGGGSGGG

SEQ ID NO: 35 - protein, 9 aa, Artificial Sequence
  Linker
GGGGSGGGG

SEQ ID NO: 36 - protein, 10 aa, Artificial Sequence
  Linker
GGGGSGGGGS

SEQ ID NO: 37 - protein, 6 aa, Artificial Sequence
  Linker
GGSGGS

SEQ ID NO: 38 - protein, 7 aa, Artificial Sequence
  Linker
GGSGGSG

SEQ ID NO: 39 - protein, 8 aa, Artificial Sequence
  Linker
GGSGGSGG

SEQ ID NO: 40 - protein, 9 aa, Artificial Sequence
  Linker
GGSGGSGGG

SEQ ID NO: 41 - protein, 10 aa, Artificial Sequence
  Linker
GGSGGSGGGG

SEQ ID NO: 42 - protein, 11 aa, Artificial Sequence
  Linker
GGSGGSGGGGS

SEQ ID NO: 43 - protein, 5 aa, Artificial Sequence
  Linker
GGGSG

SEQ ID NO: 44 - protein, 6 aa, Artificial Sequence
  Linker
GGGSGG

SEQ ID NO: 45 - protein, 7 aa, Artificial Sequence
  Linker
GGGSGGG

SEQ ID NO: 46 - protein, 8 aa, Artificial Sequence
  Linker
GGGSGGGG

SEQ ID NO: 47 - protein, 9 aa, Artificial Sequence
  Linker
GGGSGGGGG

SEQ ID NO: 48 - protein, 10 aa, Artificial Sequence
  Linker
GGGSGGGGGS

SEQ ID NO: 49 - DNA, 26 bp, Artificial Sequence
  Primer
cggaattcatgggccagactgttacc

SEQ ID NO: 50 - DNA, 18 bp, Artificial Sequence
  Primer
agcaactggcgatagtgg

SEQ ID NO: 51 - DNA, 24 bp, Artificial Sequence
  Primer
cggaattcttagggggcctcgcgg

SEQ ID NO: 52 - DNA, 18 bp, Artificial Sequence
  Primer
actacatgctgaaccggg

SEQ ID NO: 53 - protein, 30 aa, Artificial Sequence
  Peptide
PLVSSQCVNLTTRTQLPPAYTNSFTRGVYY

SEQ ID NO: 54 - protein, 30 aa, Artificial Sequence
  Peptide
RGVYYPDKVFRSSVLHSTQDLFLPFFSNVT

SEQ ID NO: 55 - protein, 30 aa, Artificial Sequence
  Peptide
FSNVTWFHAIHVSGTNGTKRFDNPVLPFND

SEQ ID NO: 56 - protein, 29 aa, Artificial Sequence
  Peptide
LPFNDGVYFASTEKSNIIRGWIFGTTLDS

SEQ ID NO: 57 - protein, 30 aa, Artificial Sequence
  Peptide
TLDSKTQSLLIVNNATNVVIKVCEFQFCND

SEQ ID NO: 58 - protein, 30 aa, Artificial Sequence
  Peptide
QFCNDPFLGVYYHKNNKSWMESEFRVYSSA

SEQ ID NO: 59 - protein, 30 aa, Artificial Sequence
  Peptide
VYSSANNCTFEYVSQPFLMDLEGKQGNFKN

SEQ ID NO: 60 - protein, 30 aa, Artificial Sequence
  Peptide
GNFKNLREFVFKNIDGYFKIYSKHTPINLV

SEQ ID NO: 61 - protein, 29 aa, Artificial Sequence
  Peptide
PINLVRDLPQGFSALEPLVDLPIGINITR

SEQ ID NO: 62 - protein, 30 aa, Artificial Sequence
  Peptide
NITRFQTLLALHRSYLTPGDSSSGWTAGAA

SEQ ID NO: 63 - protein, 30 aa, Artificial Sequence
  Peptide
TAGAAAYYVGYLQPRTFLLKYNENGTITDA

SEQ ID NO: 64 - protein, 30 aa, Artificial Sequence
  Peptide
TITDAVDCALDPLSETKCTLKSFTVEKGIY

SEQ ID NO: 65 - protein, 28 aa, Artificial Sequence
  Peptide
EKGIYQTSNFRVQPTESIVRFPNITNLC

SEQ ID NO: 66 - protein, 25 aa, Artificial Sequence
  Peptide
FNATRFASVYAWNRKRISNCVADYS

SEQ ID NO: 67 - protein, 25 aa, Artificial Sequence
  Peptide
VADYSVLYNSASFSTFKCYGVSPTK

SEQ ID NO: 68 - protein, 30 aa, Artificial Sequence
  Peptide
VSPTKLNDLCFTNVYADSFVIRGDEVRQIA

SEQ ID NO: 69 - protein, 30 aa, Artificial Sequence
  Peptide
VRQIAPGQTGKIADYNYKLPDDFTGCVIAW

SEQ ID NO: 70 - protein, 30 aa, Artificial Sequence
  Peptide
CVIAWNSNNLDSKVGGNYNYLYRLFRKSNL

SEQ ID NO: 71 - protein, 25 aa, Artificial Sequence
  Peptide
KPFERDISTEIYQAGSTPCNGVEGF

SEQ ID NO: 72 - protein, 30 aa, Artificial Sequence
  Peptide
GVEGFNCYFPLQSYGFQPTNGVGYQPYRVV

SEQ ID NO: 73 - protein, 30 aa, Artificial Sequence
  Peptide
PYRVVVLSFELLHAPATVCGPKKSTNLVKN

SEQ ID NO: 74 - protein, 29 aa, Artificial Sequence
  Peptide
NLVKNKCVNFNFNGLTGTGVLTESNKKFL

SEQ ID NO: 75 - protein, 28 aa, Artificial Sequence
  Peptide
PFQQFGRDIADTTDAVRDPQTLEILDIT

SEQ ID NO: 76 - protein, 29 aa, Artificial Sequence
  Peptide
ILDITPCSFGGVSVITPGTNTSNQVAVLY

SEQ ID NO: 77 - protein, 30 aa, Artificial Sequence
  Peptide
AVLYQDVNCTEVPVAIHADQLTPTWRVYST

SEQ ID NO: 78 - protein, 30 aa, Artificial Sequence
  Peptide
RVYSTGSNVFQTRAGCLIGAEHVNNSYECD

SEQ ID NO: 79 - protein, 30 aa, Artificial Sequence
  Peptide
SYECDIPIGAGICASYQTQTNSPRRARSVA

SEQ ID NO: 80 - protein, 30 aa, Artificial Sequence
  Peptide
TMSLGAENSVAYSNNSIANNSIAIPTNFTI

SEQ ID NO: 81 - protein, 30 aa, Artificial Sequence
  Peptide
TSVDCTMYICGDSTECSNLLLQYGSFCTQL

SEQ ID NO: 82 - protein, 30 aa, Artificial Sequence
  Peptide
FCTQLNRALTGIAVEQDKNTQEVFAQVKQI

SEQ ID NO: 83 - protein, 28 aa, Artificial Sequence
  Peptide
QVKQIYKTPPIKDFGGFNFSQILPDPSK

SEQ ID NO: 84 - protein, 30 aa, Artificial Sequence
  Peptide
PDPSKPSKRSFIEDLLFNKVTLADAGFIKQ

SEQ ID NO: 85 - protein, 30 aa, Artificial Sequence
  Peptide
GFIKQYGDCLGDIAARDLICAQKFNGLTVL

SEQ ID NO: 86 - protein, 21 aa, Artificial Sequence
  Peptide
TDEMIAQYTSALLAGTITSGW

SEQ ID NO: 87 - protein, 30 aa, Artificial Sequence
  Peptide
ITSGWTFGAGAALQIPFAMQMAYRFNGIGV

SEQ ID NO: 88 - protein, 30 aa, Artificial Sequence
  Peptide
NGIGVTQNVLYENQKLIANQFNSAIGKIQD

SEQ ID NO: 89 - protein, 30 aa, Artificial Sequence
  Peptide
GKIQDSLSSTASALGKLQDVVNQNAQALNT

SEQ ID NO: 90 - protein, 28 aa, Artificial Sequence
  Peptide
LNTLVKQLSSNFGAISSVLNDILSRLDK

SEQ ID NO: 91 - protein, 28 aa, Artificial Sequence
  Peptide
LDKVEAEVQIDRLITGRLQSLQTYVTQQ

SEQ ID NO: 92 - protein, 28 aa, Artificial Sequence
  Peptide
YVTQQLIRAAEIRASANLAATKMSECVL

SEQ ID NO: 93 - protein, 27 aa, Artificial Sequence
  Peptide
CVLGQSKRVDFCGKGYHLMSFPQSAPH

SEQ ID NO: 94 - protein, 30 aa, Artificial Sequence
  Peptide
PHGVVFLHVTYVPAQEKNFTTAPAICHDGK

SEQ ID NO: 95 - protein, 30 aa, Artificial Sequence
  Peptide
CHDGKAHFPREGVFVSNGTHWFVTQRNFYE

SEQ ID NO: 96 - protein, 20 aa, Artificial Sequence
  Peptide
RNFYEPQIITTDNTFVSGNC

SEQ ID NO: 97 - protein, 27 aa, Artificial Sequence
  Peptide
NNTVYDPLQPELDSFKEELDKYFKNHT

SEQ ID NO: 98 - protein, 28 aa, Artificial Sequence
  Peptide
FKNHTSPDVDLGDISGINASVVNIQKEI

SEQ ID NO: 99 - protein, 808 aa, Artificial Sequence
  RBD fusion protein
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPHHHHHH

SEQ ID NO: 100 - protein, 557 aa, Artificial Sequence
  RBD fusion protein
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPGGWSSKPRKGMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYFPAGGSSSGTVNPAPNIASHISSISARTGDPVTNPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 101 - protein, 20 aa, Artificial Sequence
  Signal Peptide
MYRMQLLSCIALSLALVTNS

(19) United States
(12) Patent Application Publication
(10) Pub. No.: US 2025/0345413 A1
(43) Pub. Date: Nov. 13, 2025

(54) SARS-COV-2 SUBUNIT VACCINE

(71) Applicant: VIRAVAXX AG, Vienna (AT)

(72) Inventors: Pia GATTINGER, Vienna (AT); Rudolf VALENTA, Theresienfeld (AT); Bernhard KRATZER, Schäffern (AT); Winfried PICKL, Vienna (AT); Frank STOLZ, Vienna (AT)

(73) Assignee: VIRAVAXX AG, Vienna (AT)

(21) Appl. No.: 18/282,689

(22) PCT Filed: Mar. 29, 2022
(86) PCT No.: PCT/EP2022/058280

§ 371 Date: Sep. 18, 2023

(30) Foreign Application Priority Data
Mar. 30, 2021 (EP) 21165982.6

(51) Int. Cl. A61K 39/215; A61K 39/00; A61P 31/14

(52) U.S. Cl. CPC A61K 39/215; A61P 31/14; A61K 2039/5258; A61K 2039/6075

(57) ABSTRACT

An immunogenic subunit vaccine antigen which comprises at least two receptor-binding domains (RBDs) of the spike (S) protein of SARS-CoV-2 which are fused to a heterologous immunogenic carrier protein, wherein each of said at least two RBDs has a folded structure in an accessible conformation to bind the human angiotensin-converting enzyme 2 (ACE2) receptor protein.

### FIELD OF THE INVENTION

The present invention relates to novel vaccine antigens and vaccines, for preventing SARS-CoV-2 infections.

### BACKGROUND OF THE INVENTION

The causative agent of COVID-19, SARS-CoV-2, is a β-corona virus related phylogenetically to previously identified pathogenic agents that cause fatal respiratory disease in humans, severe acute respiratory syndrome virus SARS-CoV and Middle East respiratory syndrome virus, MERS (MERS-CoV). Coronaviruses in general are responsible for substantial human and animal morbidity and mortality and the potential for continued emergence of novel pathogens from this class is highlighted by the relatively rapid appearance of three highly severe human diseases within two decades. SARS-CoV-2 binds to and enters human cells through an interaction between the RBD of the S protein to angiotensin-converting enzyme 2 (ACE2). Potent neutralizing monoclonal antibodies against multiple epitopes on S have been isolated from convalescent patients and recent studies have shown that human antibodies can be effective for the treatment of COVID-19.

SARS-CoV and SARS-CoV-2 use angiotensin-converting enzyme 2 (ACE2) on human cells as receptor and bind to it with their receptor binding domain (RBD). The RBD is located in the spike (S) protein within S1, the receptor-binding subunit close to the C-terminal S2 membrane fusion subunit.

For certain viral diseases (e.g., respiratory syncytial virus, RSV) folded viral surface antigens or immunogens mimicking the conformation of the natural and folded antigen are required for inducing neutralizing antibodies. For other viruses (e.g., hepatitis B, HBV) unfolded surface antigens have been found to induce protective antibodies and virus attachment can be blocked with unfolded peptides derived from the viral receptor binding site. For SARS-CoV-2 it is not yet known if antibodies towards sequential or conformational epitopes or both determine the neutralizing activity of the natural polyclonal antibody response. Likewise, it has not been known if one can induce protective antibodies with a SARS-CoV-2 vaccine based on sequential and/or unfolded antigens, or if the vaccine needs to contain folded SARS-CoV-2 antigens, in particular RBD. For example, for SARS-CoV it has been reported that potent neutralizing antibodies and protective immunity can be obtained by immunization with RBD expressed in a folded form in eukaryotic cells as well as with unfolded RBD, Escherichia coli-expressed RBD (Du, L., et al. Virology. 2009; 393:144-150). These results were consistent with data obtained for several vaccines for other infectious diseases and therapeutic vaccines for allergy demonstrating that one can induce protective antibody responses against the corresponding natural, folded antigen resembling conformational epitopes with the denatured antigens, the unfolded recombinant antigen or sequential peptides thereof (Cornelius C. et al. EBioMedicine. 2016; 11:58-67; Tulaeva I. et al. EBioMedicine. 2020; 59: 102953; Ni Y., et al. Gastroenterology. 2014; 146:1070-83; Volkman, D. J. et al. J Immunol. 1982; 129:107-112; Sela, M. & Arnon, R, Vaccine. 1992; 10:991-999; Marsh, D. G. et al. Immunology. 1970; 18:705-722; Valenta, R. Nat Rev Immunol. 2002; 2:446-453). Conversely, it has been suggested for certain viral diseases that immunization with correctly folded antigens is required for obtaining protective antibody responses (McLellan, J. S., et al. Science. 2013; 342:592-598; Sesterhenn, F., et al. Science. 2020; 368(6492):eaay5051).

It has been shown that COVID-19 patients develop SARS-CoV-2-specific antibodies but it is not known if and in how many infected subjects the virus-induced antibodies are protective. Indeed, it was reported that patients who had recovered from COVID-19 again presented with detectable positive SARS-CoV-2 RNA (Fu et al., J Med Virol. 2020; 92(11):2298-2301).
Dai et al. (Cell 2020, 182:722-733) describe CoV RBD-dimer immunogens comprised of two protein subunits each containing the virus spike receptor binding domain fused together via a disulfide link or as tandem repeat single chain (sc) without introducing any exogenous sequence (RBD-sc-dimers). RBD-sc-dimers of MERS-CoV and SARS-CoV-2 were expressed in CHO cells.
Du et al. (Virology 2009, 393:144-150) describe a recombinant receptor-binding domain of SARS-CoV spike protein expressed in mammalian, insect and E. coli cells to elicit a neutralizing antibody response.

An enzyme-linked immunosorbent assay (ELISA) for detecting neutralizing antibodies against SARS-CoV-2 has been described in Tan et al. (Nature Research 2020, doi: 10.21203/rs.3.rs-24574/v1 Preprint). The test is described to identify subjects producing antibodies which inhibit the binding of the receptor binding domain (RBD) of SARS-CoV-2 to its receptor ACE2 on human cells.
Gattinger et al. (Allergy. 2021; 76(3):878-883) describe a molecular RBD-ACE2 interaction assay which could be useful for identifying subjects having developed protective antibodies and for screening candidate vaccines to induce antibodies that inhibit the RBD-ACE2 interaction.
Quinlan et al. (bioRxiv. 2020; 2020.11.18.388934. doi: 10.1101/2020.11.18.388934. Preprint) disclose RBD conjugated to two carrier proteins that elicited more potent neutralizing responses in immunized rodents than did a similarly conjugated proline-stabilized S protein ectodomain. A glyco-engineered RBD expressed more efficiently, and generated a more potent neutralizing responses as a DNA vaccine antigen, than the wild-type RBD or the full-length S protein, especially when fused to multivalent carriers such as an H. pylori ferritin 24-mer. However, this study only compared different immunogens but not different versions of RBD.
WO 2017/037280 discloses fusion proteins comprising a hepatitis B (HBV) PreS polypeptide for use in the treatment of HBV virus infection but PreS described in this application has been expressed in Escherichia coli as unfolded protein (Cornelius C. et al. EBioMedicine. 2016; 11:58-67).
Sun Shihui et al. (Cellular & Molecular Immunology 2021, 18(4): 1070-1073) describe an RBD-Fc fusion for use as a subunit vaccine. An RBD domain (aa331-524) is fused to the human IgG1 Fc fragment. Two fusion polypeptides, each containing one RBD fused to the human IgG1 Fc fragment form a dimer via the Fc fragments. Thus two RBD domains are fused through the Fc fragment to form a dimer with a Y-shaped structure, like an antibody.
CN 111944064A discloses a COVID-19 subunit vaccine comprising a trimer and/or dimer and/or monomer of a fusion protein comprising from the N-end to the C-end, human interleukin 10 signal peptide, S-S-RBD, and foldon protein. The dimer/trimer formation occurs by disulfide bridges.
CN 111533809A discloses a fusion protein composed of one RBD structure domain of SARS-CoV-2 protein S and the Fc segment of a human IgG1 antibody.
Yang Shilong et al. (The Lancet Infectious Diseases 2021, 21(8): 1107-1119) describe a tandem-repeat dimeric RBD-based protein subunit vaccine used in clinical trials.
Dai et al. (Cell 2020, 182:722-733) describe a vaccine design using a RBD-dimer as a tandem repeat single-chain.
Jeong Hyein et al. (Frontiers in Immunology 2021, 12:637654) describe a DNA vaccine encoding a chimeric protein of RBD fused to the 11 aa long N-terminal region of Hepatis preS1 with a W4P mutation.
WO2014134439 A1 discloses an immunogenic composition for MERS coronavirus infection, comprising at least a portion of MERS-CoV S protein and an immunopotentiator.

There is a need for effective vaccines inducing protective immunity against SARS-CoV-2. In particular there is a need for SARS-CoV-2 vaccines which induce high levels of RBD-specific antibodies that inhibit the binding of the virus to its receptor on host cells (ACE2) which can be used for repeated booster injections to maintain high levels of antibodies conferring sterilizing immunity.

### SUMMARY OF THE INVENTION

It is the objective of the present invention to provide new vaccine antigens to trigger a protective antibody immune response against SARS-CoV-2. The objective is solved by the subject of the present claims and as further described herein.

In the current invention antibody responses obtained by immunization were compared with folded versus unfolded RBD and their virus-neutralizing activity. For this purpose, rabbits were immunized with a folded and unfolded recombinant RBD protein. Surprisingly and in contrast to SARS-CoV we found that only immunization with folded but not with unfolded RBD induced antibodies against conformational RBD epitopes and high virus neutralizing titers (VNTs).

Collectively, the present data demonstrate that the virus-neutralizing activity of antibodies in COVID-19 patients depends on the presence of antibodies directed to conformational epitopes of RBD. However, not all COVID-19 patients develop these antibodies. Importantly, the induction of such antibodies by vaccination requires folded RBD. Thus, the present results suggest that antibodies against conformational RBD epitopes are a surrogate marker for a SARS-CoV-2 neutralizing antibody response and are important for the development of SARS-CoV-2-specific vaccines capable of inducing sterilizing immunity. In the current invention SARS-CoV-2 vaccine candidates are described based on folded RBD which are capable of inducing high levels of neutralizing antibody titers and their advantages. These vaccine candidates have the advantage that they induce higher levels of protective antibodies as vaccines based on isolated RBD or RBD dimers (Dai et al. Cell. 2020; 182:722-733) and/or offer the induction of additional protective antibodies against other viral infections.

The vaccine candidates advantageously use an immunogenic carrier protein which is heterologous to the subject receiving the vaccine. Thus, in a setting of vaccinating human subjects, the heterologous carrier protein is particularly non-human, and immunogenic in the human subject. This avoids complications of undesired autoimmune reactions. The heterologous carrier protein advantageously comprises T cell epitopes and B cell epitopes. Exemplary carrier proteins are of viral origin, such as nucleocapsid proteins or preS proteins, or protein domains thereof. Such immunogenic carrier proteins have been tested in animal models and turned out to effectively improve immunogenicity of the RBD units being fused to the carrier protein. Following vaccination of a human subject with an exemplary vaccine described herein, it has been proven that the respective anti-SARS-CoV-2 immune response not only was directed to the SARS-CoV-2 virus that comprised the RBD unit as used in the vaccine antigen, but also to variants thereof (including variants of concern), such as e.g., the Omicron variant.

In particular, the present disclosure refers to the construction and characterization of a SARS-CoV-2 subunit vaccine antigen comprising a single-chain fusion protein (“PreS-RBD”) based on a structurally folded recombinant fusion protein consisting of two SARS-CoV-2 Spike protein receptor binding domains (RBD) fused to the N- and C-terminus of hepatitis B virus (HBV) surface antigen PreS to enable that two unrelated proteins serve as immunologic carriers for each other. PreS-RBD, but not RBD or RBD dimer alone, induced a robust and uniform RBD-specific IgG response in rabbits. Currently available genetic SARS-CoV-2 vaccines induce mainly transient IgG₁ responses in vaccinated subjects. Advantageously, the PreS-RBD vaccine was found to induce RBD-specific IgG antibodies consisting of an early IgG₁ and sustained IgG4 antibody response in a SARS-CoV-2 naïve human subject. PreS-RBD-specific IgG antibodies were detected in serum and mucosal secretions, reacted with SARS-CoV-2 variants, including the omicron variant of concern and the HBV receptor binding sites on PreS of currently known HBV-genotypes. PreS-RBD-specific antibodies of the immunized subject more potently inhibited the interaction of RBD with its human receptor ACE2 and their VNTs were higher than median VNTs in a random sample of healthy subjects fully immunized with registered SARS-CoV-2 vaccines or in COVID-19 convalescent subjects. Thus, the PreS-RBD vaccine has the potential to serve as a combination vaccine for inducing sterilizing immunity against SARS-CoV-2 and HBV by stopping viral replication through the inhibition of cellular virus entry.

PreS-RBD was formulated with aluminum hydroxide (alum), an adjuvant which has been safely used both in vaccines against infectious diseases and in therapeutic allergy vaccines (i.e., allergen-specific immunotherapy, AIT) for decades. AIT-induced allergen-specific IgG responses typically consist of rapidly evolving specific IgG₁ responses and the late but sustained production of neutralizing allergen-specific IgG₄ antibodies, which persist even years after discontinuation of treatment and leads to sustained protection of allergic patients from allergen-induced allergic inflammation. Results obtained for the PreS-RBD subunit vaccine in the exemplary study described herein suggest that PreS-RBD has several features, which make it a promising SARS-CoV-2 vaccine candidate for inducing sterilizing immunity.

The present invention provides for an immunogenic subunit vaccine antigen which comprises at least two receptor-binding domains (RBDs) of the spike (S) protein of SARS-CoV-2 which are fused to a heterologous protein, wherein each of said at least two RBDs has a folded structure in an accessible conformation to bind the human SARS-CoV-2 receptor, i.e., the angiotensin-converting enzyme 2 (ACE2) protein. In particular, the heterologous protein is an immunogenic carrier protein.

Herein, the term “heterologous immunogenic carrier protein” is also used in the abbreviated form, as “heterologous protein”. Therefore, it is understood that the present disclosure of a “heterologous protein” shall specifically also refer to the “heterologous immunogenic carrier protein”.

Specifically, the immunogenic carrier protein is immunogenic in a human subject.

Specifically, the heterologous immunogenic carrier protein is an antigen comprising B cell epitopes and T cell epitopes to elicit humoral and cellular immune responses in a human subject.

Specifically, the immunogenic carrier protein is a non-human protein, or an artificial protein, such as e.g., a mutant of a non-human protein. Specific immunogenic carrier proteins are viral proteins, viral protein domains or substructures thereof, preferably comprising T cell and B cell epitopes.

Specifically, the heterologous immunogenic carrier protein is different from, or any other than an RBD of the spike (S) protein of SARS-CoV-2. Specifically, the heterologous immunogenic carrier protein is a viral protein or a domain of a viral protein, except the RBD domain of SARS-CoV-2.

It is specifically preferred that the heterologous immunogenic carrier protein is not a human protein, such as e.g., an antibody, or an antibody fragment thereof, like a human antibody Fc domain, or a human cytokine, interleukin, or fragments thereof.

Specifically, the RBD has a folded structure and is understood as “folded RBD”, such as further described herein.

Specifically, the vaccine antigen is a fusion protein comprising at least two receptor-binding domains (RBDs) of the spike (S) protein of SARS-CoV-2 which are fused to a heterologous immunogenic carrier protein, wherein each of said at least two RBDs has a folded structure in an accessible conformation to bind the human angiotensin-converting enzyme 2 (ACE2) protein.

Specifically, the at least two RBDs are composed of or include an RBD dimer which consists of two RBDs, an RBD trimer which consists of three RBDs, or an RBD oligomer which consists of four or more, preferably 4-8 RBDs.

Specifically, the RBDs included in the RBD dimer, trimer or oligomer are herein also referred to as RBD protomers. RBD protomers may comprise or consist of an identical RBD sequence, in particular over the full length of the RBD (i.e., be identical), also referred to as a symmetric dimer, trimer, or oligomer of RBD protomers. Alternatively, the RBD protomers comprised in the RBD dimer, trimer or oligomer, may differ in sequence, which is also referred to as an asymmetric dimer, trimer, or oligomer of RBD protomers.

According to a specific aspect, the RBDs included in the RBD dimer, trimer or oligomer may be comprised in only one fusion protein, in particular in a single chain fusion protein, wherein an RBD protomer is fused to another part of the fusion protein such that the C-terminus of the RBD protomer is fused to the N-terminus of the other part (with or without using a linker); or such that the N-terminus of the RBD protomer is fused to the C-terminus of the other part (with or without using a linker). Such fusion is understood as a fusion “in tandem”.

Specifically, at least two RBDs are comprised in a fusion protein comprising said RBDs fused to a heterologous immunogenic carrier protein as a single-chain fusion protein, preferably comprising one or more peptide linker sequences.

Specifically, the vaccine antigen is provided as a single-chain fusion protein comprising said at least two RBDs fused to said heterologous immunogenic carrier protein, preferably comprising one or more peptide linker sequences.

According to another specific aspect, the RBDs included in the RBD dimer, trimer or oligomer may be comprised in more than one fusion proteins, in particular wherein one or more of the RBD protomers are fused to a first heterologous protein, and one or more further RBD protomers are fused to a second heterologous protein (which first and second heterologous proteins may be copies of the same protein, or may differ from one another), such that the first and second heterologous protein display the RBDs which are fused to the respective first and second heterologous proteins in close proximity to each other, thereby obtaining an assembly of the fused RBDs comprising at least two RBDs. The assembly of RBDs is herein also referred to as a complex, or a non-fused assembly of RBD protomers, such as a non-fused dimer, trimer or oligomer. The complex specifically comprises the RBDs with parallel topology e.g., axial symmetry, in particular comprising a side-to-side dimer interface of the protomers.

According to a specific aspect, the vaccine antigen comprises at least two RBDs that are each fused to an anchor protein that displays said RBDs on the surface of a virus-like particle (VLP). Specifically, said RBDs and/or a respective RBD assembly bound to the surface of the a VLP can be determined by electron microscopy.

Specifically, said at least two RBDs consist of the same or different amino acid sequence. Specific examples comprise a diversity of RBD protomers, wherein the RBDs originate from different variants of SARS-CoV-2.

According to a specific aspect, at least one, or at least two of said RBDs, each comprises or consists of an amino acid sequence of at least any one of 180, 181, 182, 183, 184, 185, 186, 187, 188, 189, 190, 191,192, 193, 194, 195, 195 197, 198, 199, or 200 amino acids length, or more than 200 aa, e.g., up to 254 aa, which originates from the amino acid sequence of the SARS-CoV-2 S protein, such as identified as Protein ID.: GenBank: QHR63270.2, or which is even longer, such as to comprise at least part of the C-terminal extension identified as SEQ ID NO:3 e.g., comprising at least the RBD part of amino acids 318-571 from QHR63270.2 (counted without leader from S protein).

Specifically, at least one, or at least two, three, or each of said RBDs comprises or consists of at least any one of 95%, 96%, 97%, 98%, 99%, or 100% sequence identity to SEQ ID NO:1, with or without a C-terminal extension comprising at least part, or all of SEQ ID NO:3 as C-terminal extension to SEQ ID NO:1, which SEQ ID NO:1 is herein also referred to as a natural RBD sequence of SARS-CoV-2.

Specifically, at least one, or at least two, three, or each of said RBDs comprises or consists of at least any one of 95%, 96%, 97%, 98%, 99%, or 100% sequence identity to SEQ ID NO:2, which comprises the natural RBD sequence of SARS-CoV-2.

A natural RBD sequence of SARS-CoV-2 may undergo mutagenesis to comprise one or more, preferably a limited number of e.g., up to 20, or less, such as up to 19, 18, 17, 16, 15, 14, 13, 12, 11, ten, or nine, or eight, or seven, or six, or five, or four, or three, or two point mutations, or comprising no more than one point mutation.

Specifically, one or more of said point mutations, or each of said one or more of point mutations, are the same as comprised in an RBD of one or more different naturally-occurring SARS-CoV-2 mutants, or the same as comprised in one or more different RBDs (e.g., a variety of RBDs) of naturally-occurring SARS-CoV-2 mutants.

Specifically, one or more of the point mutations contained in the RBD sequence are selected from the group consisting of N501Y, E484K, and K417N. Specifically, one, two or all three of N501Y, E484K, and K417N may be contained in the RBD sequence.

Specifically, unless indicated otherwise, numbering of aa positions provided herein is according to the sequence of the respective region of the SARS-CoV-2 RBD (SEQ ID NO:1, 1-192 aa, or SEQ ID NO:2, 1-254 aa).

The number of point mutations as compared to the natural RBD sequence may be increased e.g., to cover any and all relevant naturally-occurring RBD point mutations of SARS-CoV-2 mutant, such that an RBD comprised in the vaccine antigen disclosed herein elicits a cross-reactive immune response to cover any and all of the respective mutants and those which may arise from recombination of such mutations. Specifically, the selection of point mutations is to cover mutants which are already naturally-occurring, or which may naturally evolve upon mutagenesis.

An exemplary naturally-occurring SARS-CoV-2 mutant may comprise an RBD, which comprises or consists of an amino acid sequence identified as any one of SEQ ID NO:4, 5, 6, or 7. Another exemplary naturally-occurring SARS-CoV-2 mutant may comprise an RBD comprising mutations as occurring in one or more SARS-CoV-2 variants designated by the WHO, such as e.g., new variant B.1.1.529, designated variant of concern (VOC), Omicron.

Specifically, the RBD has a folded structure and a respective conformation to present one or more conformational epitopes recognized by SARS-CoV-2 neutralizing antibodies.

According to a specific aspect, the folded structure of the RBD is
a) obtained by expression of the vaccine antigen in a recombinant eukaryotic expression system, preferably employing mammalian (such as e.g., human or hamster, like CHO cells), baculovirus-infected insect cells, or fungal cells, such as e.g., yeast or filamentous fungi, host cells; and/or
b) determined by circular dichroism (CD) spectroscopy and/or an RBD-ACE2 interaction assay.

Specifically, the RBDs have a folded structure in an accessible conformation to bind hACE2, as determined by an RBD-ACE2 interaction assay such as employing a respective immunoassay or ELISA.

According to a specific aspect, the folded RBDs and/or the vaccine antigen as described herein are recognized by anti-SARS-CoV-2 antibodies and the respective antibody preparations, such as those comprising serum or antibodies from COVID-19 convalescent patients, or a respective monoclonal antibody preparation, which antibodies block (or inhibit) the binding of RBD to ACE2 in the described RBD-ACE2 interaction assay by at least any one of 20%, 30%, 40%, or preferably at least any one of 50%, 60%, 70%, 80%, 90%, or completely (100% inhibition). Specifically, inhibition of binding of RBD to ACE2 is determined in the presence of any such antibody preparations which comprise a virus neutralization titer of at least any one of 1:50, 1:60, 1:70, 1:80, 1:90, preferably at least 1:100.

Specifically, the folded RBDs and/or the vaccine antigen as described herein is competing with any neutralizing anti-SARS-CoV-2 antibody preparation in the RBD-ACE2 interaction assay.

Specifically, the folded RBD structure is in a pre-fusion conformation.

Specifically, the folded RBD structure can be determined by far-UV circular dichroism (CD) spectroscopy. Specifically, the folded RBD may or may not comprise one or more intramolecular disulfide bonds that stabilize the RBD fold. Specifically, one or more intramolecular disulfide bonds can stabilize one or more of an alpha-helix structure and/or beta-sheet structure of the RBD e.g., 1, 2, 3, or 4 disulfide bonds, such as occurring in a natural RBD fold, and/or in particular within the RBD core and/or RBD beta-sheet regions and/or connecting the loops at the distal end of the respective receptor binding motif (RBM).

Specifically, the antigen comprises or consists of a recombinant polypeptide which is produced by recombinant expression techniques employing recombinant host cells and conditions that allow the expression or manufacturing of RBD in the folded form.

Specific recombinant host cells provide for a folded structure of RBD in a pre-fusion conformation. Such host cells are preferably eukaryotic host cells, in particular mammalian host cells such as used in mammalian expression systems, for example, employing human, non-human primate, or rodent, such as hamster or mouse, cell lines.

Specific preferred host cells are e.g., HEK293 cells, CHO cells, NS0 cells, Sf9 cells, High Five cells, Pichia pastoris, Saccharomyces cerevisiae, among many others.

Specifically, the pre-fusion conformation comprises a conformational structure and the respective conformational epitopes as comprised in the viral protein before its fusion to a target cell or a cellular receptor.

The folded structure of an RBD, and particularly its pre-fusion conformation, comprises a structure where specific regions within the RBD are accessible to binding the receptor protein ACE2, and other regions are buried in the RBD folded structure.

Specifically, functionality of the folded RBD can be determined by binding the RBD to its receptor hACE2, such as determined in a respective ACE2 binding assay, or an RBD-hACE2 interaction assay, or in a BIACORE assay. A suitable RBD-ACE2 interaction assay is described in Gattinger et al. (Allergy. 2021; 76(3):878-883), or in the Examples section below.

According to a specific example, the RBD-ACE2 interaction assay is an assay, such as a binding assay determining binding of RBD to its receptor ACE2, employing
a) an ACE2 protein, and
b) a SARS-CoV-2 polypeptide comprising or consisting of a natural RBD, in particular folded RBD;
c) at least one detection molecule or labelling molecule, such as to allow quantifying the amount of binding of a) to b); and
d) optionally a solid support immobilizing a) or b).

Specifically, the ACE2 protein is human ACE2 or a functional fragment thereof which is capable of recognizing and specifically binding to natural RBD. Human ACE2 is specifically characterized by comprising or consisting of the amino acid sequence identified as SEQ ID NO:30, Uniprot:Q9BYF1.

Specifically, the RBD-ACE2 interaction assay comprises the steps:
a) incubating the human ACE2 protein (or the functional fragment thereof capable of recognizing a natural RBD) with the SARS-CoV-2 protein comprising or consisting of a natural RBD, to determine the RBD-ACE2 interaction; and
b) comparing the RBD-ACE2 interaction in the presence of an RBD containing compound, wherein an interference or reduction of the RBD-ACE2 interaction in the presence of the RBD containing compound indicates an RBD fold that is suitably used in the vaccine antigen.

By using an RBD-ACE2 interaction assay, the RBD folded structure can be verified, if it inhibits the virus-receptor binding in a competitive way.

A compound is determined to interfere or inhibit RBD-ACE2 binding when a reduced binding level is determined (e.g., when the respective reduction of the binding levels is more than 5%, preferably more than 10%) in the presence of said compound when compared to the binding level determined in the presence of a lower amount of said compound, or in the absence of said compound. An RBD that is not folded is understood as a compound that does not affect RBD-ACE2 binding in the RBD-ACE2 interaction assay, and determined by a substantially identical binding level in such assay (e.g., when the respective differences of the binding levels are within 10%, preferably within 5%) in the presence of said compound when compared to the binding level determined in the presence of a lower amount of said compound, or in the absence of said compound.

The folded structure of an RBD can also be determined by far UV circular dichroism (CD) spectroscopy, such as described in Resch et al. (Clin Exp Allergy. 2011; 41(10):1468-77), or as described in the Examples section below.

According to a specific example, the method of determining the fold of the RBD by CD spectroscopy is a standard method, such as described in the Examples section below.

According to a specific aspect, the fusion protein comprises one or more linkers, such as peptide linker sequences. Specifically, a linker is used to link said at least two RBDs and optionally a further linker is used to link said heterologous protein.

Specifically, the fusion can be in any order by a peptide bond, with or without a linker. Fusion may be achieved by recombination of nucleic acid molecules encoding the respective elements, or otherwise by synthesizing the coding nucleic acid molecules or fused polypeptide sequences.

According to a specific embodiment, the fusion protein is a single-chain (sc) fusion protein.

Specifically, the fusion protein comprises at least one or at least two, or at least three RBDs fused to the N-terminus of the heterologous protein, with or without using one or more linker.

Specifically, the fusion protein comprises at least one, or at least two, or at least three RBDs fused to the C-terminus of the heterologous protein, with or without using one or more linker.

Specifically, the fusion protein comprises at least one (or at least two, or at least three) RBD(s) fused to the N-terminus, and at least one (or at least two, or at least three) RBD(s) fused to the C-terminus of the heterologous protein, with or without using one or more linker.

According to a specific embodiment, the fusion protein comprises only one RBD fused to the N-terminus, and only one RBD fused to the C-terminus of the heterologous protein, with or without using one or more linker.

Specifically, the linker can be a linker of varying length, such as a peptide linker (also referred to as peptidic linker). Linkers can be composed of flexible residues like glycine and serine so that the adjacent peptides are free to move relative to one another. The length of the linker is variable, typically ranging between 5 and 15 amino acids. Longer linkers can be used e.g., when necessary to ensure that two adjacent elements do not sterically interfere with each other. Exemplary peptidic linker comprise or consist of a sequence of a number of G and/or S, for example comprising or consisting of any one of GGGGS (SEQ ID NO:31), GGGGSG (SEQ ID NO:32), GGGGSGG (SEQ ID NO:33), GGGGSGGG (SEQ ID NO:34), GGGGSGGGG (SEQ ID NO:35), GGGGSGGGGS (SEQ ID NO:36), or GGSGGS (SEQ ID NO:37), GGSGGSG (SEQ ID NO:38), GGSGGSGG (SEQ ID NO:39), GGSGGSGGG (SEQ ID NO:40), GGSGGSGGGG (SEQ ID NO:41), GGSGGSGGGGS (SEQ ID NO:42), or GGGSG (SEQ ID NO:43), GGGSGG (SEQ ID NO:44), GGGSGGG (SEQ ID NO:45), GGGSGGGG (SEQ ID NO:46), GGGSGGGGG (SEQ ID NO:47), or GGGSGGGGGS (SEQ ID NO:48), or a linker comprising or consisting of any one of the foregoing which comprises one or two point mutations to insert or delete an amino acid, or to substitute an amino acid by an alternative amino acid selected from the group consisting of G and S.

According to further specific examples, linker may be used that are commonly used in a single chain variable fragment (Fv) antibody construct comprising a variable heavy (VH) domain linked to a variable light (VL) domain.

According to a specific aspect, the vaccine antigen may comprise one or more peptide spacers in addition to a linker, such as to improve the structure or stability of the polypeptide.

Yet, the fusion protein described herein may comprise the elements to be fused which can be bound to each other by bioconjugation, chemical conjugation or cross-linking. For example, the vaccine antigen may comprise multimerization domains, carriers, or devices such as nanostructures or beads that are suitably used to immobilize a series of polypeptides.

According to a specific aspect, the fusion protein is provided within one polypeptide chain e.g., a polypeptide with a length of at least any one of 400, 500, 600, 700, 800, or 900 amino acid length, preferably up to any one of 1000, 1500, 2000, 2500, or 3000 amino acid length.

Specifically, the vaccine antigen comprises at least two, three, or four RBDs, which are of the same virus species or variant (or mutant) origin, or of different virus species or variants (or mutants) origin. For example, said at least two RBDs originate from different SARS-CoV-2 species or mutants, e.g., wherein at least one RBD of the vaccine antigen originates from SARS-CoV-2 and at least another one RBD of the same vaccine antigen originates from a SARS-virus different from SARS-CoV-2 e.g., SARS-CoV, or MERS.

According to a specific example, the vaccine antigen comprises two, three or more RBDs, such as provided as a dimer (wherein the number of RBDs is two), trimer (wherein the number of RBDs is three) or oligomer (wherein the number of RBDs is more than three), preferably wherein at least two or at least three of the RBDs are fused in tandem (with or without a linker), or provided within an RBD protomer assembly, preferably as RBD protomer complex. Specifically, the RBDs comprised in such dimer, trimer or oligomer are identical or differ from each other.

According to specific examples, two RBDs fused in tandem are comprised in the construct comprising or consisting of SEQ ID NO:15 (Construct 2: RBD-L-RBD, FIG. 9), and three RBDs fused in tandem are comprised in the construct comprising or consisting of SEQ ID NO:16 (Construct 3: RBD-L-RBD-L-RBD, FIG. 9), wherein “L” identifies a linker. Such constructs may or may not comprise one or more linker sequences. SEQ ID NO:15 comprises one linker sequence GGGGSGGGGS (SEQ ID NO:36), and SEQ ID NO:16 comprises two linker sequences, each characterized by the aa sequence GGGGSGGGGS (SEQ ID NO:36). The linker is fusing the C-terminus of one RBD to the N-terminus of another RBD. The tandem RBD construct may comprise or consist of the SEQ ID NO:15, or SEQ ID NO:16 e.g., including the linker sequence comprised in any such SEQ ID NO:15 or SEQ ID NO:16, or may include an alternative linker sequence, or may be provided without any linker sequence.

Either of SEQ ID NO:15 and SEQ ID NO:16 comprises a C-terminal His-tag. It is, however, understood that such constructs can be provided with or without any such tag.

Specifically, at least two RBDs that are identical to each other may be used, e.g., one or more copies of an RBD. In an RBD-dimer wherein the RBDs are identical, it is understood that the number of RBD copies in the RBD-dimer is two. In an RBD-trimer wherein the RBDs are identical, it is understood that the number of RBD copies in the RBD-trimer is three.

The parts of the fusion protein described herein are also referred to as “elements” (or “domains”), particularly wherein the elements comprise or consist of one or more of said at least two RBDs of SARS-CoV-2, and the heterologous protein.

The fusion protein may or may not comprise two or more, or all elements e.g., fused in tandem, wherein the C-terminus of a first element is fused to the N-terminus of a second element, and optionally wherein the C-terminus of the second element is fused to the N-terminus of a third element, with or without linker sequences between the elements. A fusion protein comprising or consisting of a fusion of all elements in tandem is specifically provided as a single-chain protein.

According to a specific aspect, the vaccine antigen comprises
a) at least two RBDs of SARS-CoV-2, in particular wherein said at least two RBDs may be identical or differ in at least one amino acid, including e.g., one or more point mutations that also occur in a naturally-occurring RBD of a SARS-CoV-2 mutant; and
b) at least one or two RBDs of a different virus, such as a beta-coronavirus e.g., of SARS-CoV, MERS, HCoV-OC43 or HKU1.

Specific heterologous proteins described herein may originate or be otherwise derived from viral proteins or protein domains, such as a surface protein or nucleocapsid protein, or a protein domain of any of the foregoing.

Specifically, the heterologous immunogenic carrier protein is a polypeptide or protein that is not naturally fused to RBD. Specifically, the heterologous immunogenic carrier protein is a viral protein such as a surface protein or nucleocapsid protein, or a protein domain of any of the foregoing.

According to a certain aspect, the heterologous protein may originate from the same virus or virus mutant as any one or more of said at least one RBDs of SARS-CoV-2, and fused to at least one of said RBDs in a different way or at a different position, such as to provide a “heterologous” element of the fusion protein. Exemplary fusions comprising such heterologous element are with one or more sub-domains of the S protein, M protein or nucleocapsid (NC) protein of SARS-CoV-2 e.g., including any one or more of the following protein domains or sub-domains: RBD, S1, S2, or NC. According to a specific embodiment, at least two RBDs are fused to the nucleocapsid (NC) protein of a SARS virus, such as SARS-CoV-2, SARS-CoV or MERS e.g., where at least one RBD is fused to the N-terminus of the NC protein and at least one RBD is fused to the C-terminus of NC protein. Specifically, the NC protein sequence comprises or consists of an amino acid sequence identified as SEQ ID NO:8, Uniprot:P0DTC9 (Nucleoprotein of SARS-CoV-2, UniProtKB-P0DTC9 (NCAP_SARS2); Wu F. et al., Nature 2020; 579:265-269). Suitable NC proteins are of SARS-CoV-2 (e.g., SEQ ID NO:8), the MERS-virus (e.g., SEQ ID NO:9), or of SARS-CoV (e.g., SEQ ID NO:10), or of a naturally-occurring variant or mutant of any of the foregoing.

Specific sub-domains of the S protein may comprise or consist of the region spanning aa550-580 in the S1 domain of the S-protein, or the region spanning aa676-710 around the furin cleavage site separating the S1 and S2 regions of the S-protein, or the region spanning aa929-952 in the S2 domain of the S-protein. Numbering of aa positions provided herein is according to the sequence of the respective region of the SARS-CoV-2 S-protein (SEQ ID NO:13), see also NCBI GenBank accession number QII57161.1 (human SARS-CoV-2, S-protein, SEQ ID NO:13).

According to a specific example, the heterologous element of the fusion protein comprises or consists of at least one additional RBD of SARS-CoV-2 (also referred to as heterologous RBD), thereby providing an at least trimeric structure characterized by comprising at least three RBDs (which may or may not be identical). The heterologous RBD may or may not be fused to one or more of the other RBDs in tandem. By using a heterologous RBD in an RBD-trimer, such as in a single-chain fusion protein comprising a number of RBDs fused in tandem wherein the number of RBDs is three, it has surprisingly turned out that the immune response against SARS-CoV-2 could be increased as compared to a vaccine antigen comprising a comparable RBD-dimer (wherein the number of RBDs is two) without an additional heterologous RBD.

According to a specific example using a heterologous RBD in the vaccine antigen described herein, at least three RBDs are fused in tandem. In a single-chain fusion protein, the heterologous RBD may be positioned as an N-terminal or a C-terminal protein domain, or else comprised as a non-terminal protein domain.

Specifically, the heterologous RBD may comprise or consist of a natural RBD sequence as naturally-occurring in a SARS-CoV-2 species or mutant. However, the RBD can be an artificial molecule which differs from any natural RBD e.g., comprising any one or more, or all relevant point mutations as naturally-occurring in a variety of natural RBD domains.

Specifically, the heterologous RBD has a folded structure.

Specifically, the heterologous RBD comprises or consists of an amino acid sequence that is identical to (or a copy of) any one or more or all of said at least two RBDs of SARS-CoV-2 comprised in the vaccine antigen, or which differs from any one or more or all of said at least two RBDs of SARS-CoV-2.

Specifically, the heterologous RBD is an RBD of SARS-CoV-2, SARS-CoV, MERS, such as comprising or consisting of the respective SEQ ID NO:1, 2, 11 and 12, or a derivative or mutant of any of the foregoing (the parent sequence), which comprises at least 50% (or at least any one of 60%, 70%, 80%, 85%, 90%, 95%, 96%, 97%, 98%, 99%, or 100%) of the length of the parent sequence and at least 90% (or at least any one of 91%, 92%, 93%, 94%, 95%, 96%, 97%, 98%, 99%, or 100%) sequence identity to the parent sequence, which may or may not be an artificial mutant comprising one or more point mutations wherein point mutation(s) may characterize one or more natural virus mutants, or which point mutation(s) may evolve through mutagenesis by a directed evolution approach mutagenizing the respective parent virus sequence.

According to a further aspect, the heterologous protein does not originate from the same virus or virus mutant as any one or more of said at least one RBDs of SARS-CoV-2, but from a different virus species or a naturally-occurring or artificial mutant thereof, thereby providing a heterologous element of the fusion protein. An exemplary source is one of mammalian viruses, such as a human or non-human animal virus.

According to a specific aspect, the source virus species which is the origin of any RBD and/or heterologous protein comprised in the vaccine antigen, is also the target virus, aiming to trigger an immune response against such target virus.

According to a specific aspect, SARS-CoV-2 is the origin of said at least two RBDs and the heterologous protein of the fusion protein, and at the same time be the target virus species. When providing a vaccine comprising such a vaccine antigen, the immune response covers at least the SARS-CoV-2 target virus species, wherein SARS-CoV-2 includes naturally-occurring SARS-CoV-2 including mutants thereof which may evolve during a season of infection or a pandemic, or mutants that are artificially evolved to anticipate naturally-occurring mutants.

According to another specific aspect, SARS-CoV-2 is the origin of said at least two RBDs of the fusion protein, and the heterologous protein of the fusion protein may originate from a different source, such as from a different target virus species. When providing a vaccine comprising such a vaccine antigen, the immune response covers at least SARS-CoV-2 as a first target virus species, wherein SARS-CoV-2 includes naturally-occurring SARS-CoV-2 including mutants thereof which may evolve during a season of infection or a pandemic, or mutants that are artificially evolved to anticipate naturally-occurring mutants. In addition, the immune response covers at least a second target virus species that is the source of the heterologous protein.

Specifically, the heterologous protein can be used as a carrier protein, which may or may not be immunogenic as such. An immunogenic carrier protein may be used which elicits an immune response against a pathogen that is different from SARS-CoV-2 (and optionally also different from any one or both of SARS-CoV, MERS). By using an immunogenic carrier protein, the immune response against SARS-CoV-2 could be increased.

Specific carrier proteins are selected from the group consisting of viral proteins.

According to a specific aspect, the heterologous protein originates from any one of:
a) a virus of the Hepadnaviridae family, such as a human hepatitis virus or hepatitis B virus, preferably wherein the heterologous protein is a surface protein of hepatitis B virus, such as a PreS or S protein; or
b) a beta-coronavirus, preferably any one of SARS-CoV-2, SARS-CoV, MERS, HCoV-OC43 or HKU1, preferably wherein the heterologous protein is selected from the group consisting of the S protein, or a subdomain thereof, such as an RBD, S1 or S2 domain, or a nucleocapsid (N) protein; or
c) a human rhinovirus serotype, preferably wherein the heterologous protein is a viral capsid protein such as any one of VP1, VP2, VP3, or VP4; or
d) a RSV, preferably wherein the heterologous protein is a G-protein, or central conserved region of the G-protein; or
e) a glycolipid anchor, and wherein the RBDs fused to the anchor are surface-expressed by a virus-like particle comprising a core protein of an enveloped virus, such as Moloney murine leukemia virus (MoMLV, such as further described herein), Vesicular Stomatitis Virus (VSV; such as described in Roberts et al. 1999. J. Virol.; 73(5):3723-32), HIV (such as described in DemI et al. Molecular Immunology 2005. 42(2):259-277), Ebola Virus (such as described in Swenson et al. 2005. Vaccine. 23(23):3033-3042), preferably wherein the core protein is a Gag and/or Gag-Pol protein of the respective virus, such as MoMLV Gag and/or Gag-Pol; or
f) a naturally-occurring mutant of any one of the foregoing.

The fusion protein described herein may comprise one or more heterologous proteins as heterologous element(s).

Specific heterologous proteins are HBV PreS polypeptides comprising or consisting of a polypeptide comprising at least any one of 80%, 85%, 90%, 95%, or 100% sequence identity to the natural PreS protein or one or more fragments thereof. Specific HBV PreS polypeptides may originate from (or derived from) any one of the HBV genotypes B, C, D, E, F, G or H, or a subtype thereof. Subtypes of hepatitis B viruses include A1, A2, A3, A4, A5, B1, B2, B3, B4, B5, C1, C2, C3, C4, C5, D1, D2, D3, D4, D5, F1, F2, F3 and F4 as discussed in Schaefer et al. (World J Gastroenterol. 2007; 13:14-21).

The presence of more than one hepatitis B PreS polypeptides in the fusion protein has the advantage that more antigens are presented to the immune system allowing the formation of antibodies directed to PreS. The HBV PreS polypeptides being part of the fusion protein of the present disclosure may be derived from the same HBV genotype or from different genotypes. For example, the fusion protein described herein may comprise the PreS polypeptide of HBV genotype A only or may be combined with a further PreS polypeptide derived from any one of the HBV genotypes B, C, D, E, F, G or H, or a subtype thereof.

Fragments of a PreS protein suitably used as heterologous element in the fusion protein consist preferably of at least any one of 30, 40, or 50 consecutive amino acid residues of the PreS protein sequence, preferably between aa1-70 of the hepatitis B PreS protein consisting of any one of SEQ ID NO:19-26 whereby SEQ ID NO:21-26 belong to HBV genotypes B to H, respectively. Specific fragments may comprise PreS1 and/or PreS2 of the hepatitis B PreS protein. Using PreS as a heterologous protein in a vaccine as described herein, induces antibodies that prevent HBV infection (Cornelius C. et al. EBioMedicine. 2016; 11:58-67).

According to specific examples, a heterologous carrier protein is used which comprises or consists of at least any one of 80%, 85%, 90%, 95%, or 100% sequence identity to a viral protein, preferably selected from the group consisting of:
a) any one of a Hepatitis B PreS protein or fragment thereof, such as a polypeptide comprising or consisting of any one of SEQ ID NO:19-26; or
b) a nucleocapsid of SARS-CoV-2, SARS-CoV, or MERS, such as comprising or consisting of the respective SEQ ID NO:8, 9, and 10; or
c) an RBD of SARS-CoV-2, SARS-CoV, MERS, such as comprising or consisting of the respective SEQ ID NO:1, 2, 11 and 12;
or a derivative or mutant of any of the foregoing (the parent sequence), which comprises at least 50% (or at least any one of 60%, 70%, 80%, 85%, 90%, 95%, 96%, 97%, 98%, 99%, or 100%) of the length of the parent sequence and at least 80% (or at least any one of 85%, 90%, 95%, 96%, 97%, 98%, 99%, or 100%) sequence identity to the parent sequence, which may or may not be an artificial mutant comprising one or more point mutations which point mutation(s) may characterize one or more natural virus mutants, or may evolve through mutagenesis by a directed evolution approach mutagenizing a parent virus sequence.

Specific embodiments refer to virus-like particles (VLP), herein also referred to as virus-like nanoparticles (VNP).

VLPs and VNPs are powerful platforms for multivalent antigen presentation. Several self-assembling proteins have been successfully used as scaffolds to present complex vaccine antigens on their surface. Such particles comprise noninfectious viral core particles, surrounded by a lipid envelope derived from the host cell plasma membrane. Noninfectious enveloped particles are inducible in mammalian cells by the expression of viral structural proteins (preferably Gag of MoMLV) in the absence of viral nucleic acids or envelope proteins.

Where proteins, such as RBDs or the respective fusion proteins as described herein, are bound to, integrate within or incorporated into the lipid bilayer envelope of a VLP, such that they are surface-expressed and displayed on the surface of the VLP, self-assembled viral protein complexes can be prepared. Self-assembly can provide for the RBD complex formation on the surface of the VLP, as further described herein.

Glycosylphosphatidylinositol (GPI)-anchored proteins use a posttranslational modification to link proteins to lipid bilayer membranes. The anchoring structure typically consists of both a lipid and carbohydrate portion and is highly conserved in eukaryotic organisms regarding its basic characteristics, yet highly variable in its molecular details.

According to a specific aspect, RBDs are fused to a GPI anchor and are surface-expressed by a virus-like particle comprising a core protein of an enveloped virus, such as Moloney murine leukemia virus (MoMLV), preferably wherein the core protein is MoMLV Gag and/or Gag-Pol.

According to a specific aspect, the vaccine antigen comprises:
a) a single-chain fusion protein comprising at least two RBDs fused to a Hepatitis B PreS polypeptide of at least any one of 50%, 60%, 70%, 80%, 85%, 90%, 95%, 96%, 97%, 98%, 99%, or 100% length of any one of SEQ ID NO:19-26, and comprising at least 80% sequence identity to the corresponding region of the respective SEQ ID NO:19-26; and/or
b) at least three RBDs fused in tandem, preferably wherein
   i. said at least three RBDs originate from SARS-CoV-2 and/or a naturally-occurring SARS-CoV-2 mutant, or
   ii. at least two of said RBDs originate from SARS-CoV-2 and/or a naturally-occurring SARS-CoV-2 mutant, and at least one of said RBDs originates from a beta-coronavirus that is different from SARS-CoV-2, such as SARS-CoV or MERS; and/or
c) at least two assembled RBDs which are each fused to a glycosyl phosphatidylinositol (GPI)-anchor and associated to the membrane of a virus-like particle expressed by a mammalian cell transfected with an expression plasmid encoding MoMLV gag-pol.

Specifically, when using a Hepatitis B PreS polypeptide as a heterologous protein, in particular as heterologous carrier protein, at least one or at least two RBDs are fused to the N-terminus and at least one or at least two RBDs are fused to the C-terminus of the PreS polypeptide.

According to a specific example, two RBDs are fused to a HBV PreS amino acid sequence e.g., as comprised in the construct comprising or consisting of SEQ ID NO:14 (Construct 1: RBD-PreS-RBD, FIG. 9). SEQ ID NO:14 comprises a first RBD, a PreS sequence and a second RBD, wherein the N-terminus of the PreS sequence is fused to the C-terminus of the first RBD, and the C-terminus of the PreS sequence is fused to the N-terminus of the second RBD. Alternative constructs comprising two RBDs and one PreS sequence may be produced, such that e.g., the first and second RBDs are fused in tandem, and either the N-terminus of the PreS sequence is fused to the C-terminus of the tandem RBD construct, or the C-terminus of the PreS sequence is fused to the N-terminus of the tandem RBD construct. The tandem RBD construct may comprise or consist of the SEQ ID NO:15 e.g., including the linker sequence comprised in SEQ ID NO:15, or an alternative linker sequence, or without any linker sequence.

SEQ ID NO:14 comprises no heterologous linker sequence and a C-terminal His-tag. It is, however, understood that such construct can be provided with or without any such linker sequence or His-tag. The SEQ ID NO:14 without the His-tag is identified as SEQ ID NO:100.

The invention further provides for an isolated nucleic acid molecule encoding the vaccine antigen described herein, preferably comprising a polynucleotide sequence comprising at least 95% (or at least 96%, 97%, 98%, 99%, or 100%) sequence identity to a sequence encoding any of the fusion proteins described herein. Exemplary polynucleotide sequences are codon-optimized sequences, which are optimized for recombinant expression in the respective host cell, such as SEQ ID NO:17 (which encodes Construct 1, RBD-PreS-RBD), or SEQ ID NO:18 (which encodes Construct 3: RBD-L-RBD-L-RBD), or a codon-optimized variant of any of the foregoing, which is optimized to be expressed in a specific host cell line.

A coding nucleic acid molecule, such as a cDNA, can be used for producing the vaccine antigen in vitro. A coding nucleic acid molecule, such as a RNA, can be used to produce an RNA-vaccine.

The invention further provides for expression constructs comprising the coding nucleic acid molecules, and recombinant host cells comprising such expression constructs and/or the coding nucleic acid molecules, and a method of expressing the vaccine antigen in a host cell culture.

According to a specific aspect, the invention further provides for an expression system for producing the vaccine antigen described herein in an ex vivo cell culture, by a recombinant host cell comprising the nucleic acid molecule described herein.

Suitable host cells may be selected from the group consisting of eukaryotic host cells, such as mammalian, baculovirus-infected cells, insect, or fungal cells, such as yeast or filamentous fungi, e.g., HEK293 cells, CHO cells, NS0 cells, Sf9 cells, High Five cells, Pichia pastoris, Saccharomyces cerevisiae, among many others.

Specifically, the vaccine antigen described herein or at least one or more of its elements i.e., at least two RBDs and the heterologous protein, may be glycosylated or non-glycosylated. In a preferred embodiment, the said at least two RBDs are glycosylated.

Specifically, the RBD may or may not comprise a glycosylation such as expressed by mammalian (e.g., non-human mammalian, such as hamster or mouse), or human cells e.g., HEK cells or CHO cells.

According to a specific aspect, the invention further provides for a method of producing the vaccine antigen described herein, wherein a recombinant host cell described herein is cultivated or maintained under conditions to produce said vaccine antigen.

The invention further provides for vaccine or vaccine preparation comprising the vaccine antigen described herein, or the nucleic acid molecule described herein, optionally further comprising any one or more of a pharmaceutically acceptable carriers, an excipient or an adjuvant.

Depending on the dosage, the form and administration route the vaccine antigen described herein may be combined with excipients, diluents, adjuvants and/or carriers.

Suitable protocols for the production of vaccine formulations are known to the person skilled in the art and can be found e.g., in “Vaccine Protocols” (A. Robinson, M. P. Cranage, M. Hudson; Humana Press Inc., U.S.; 2nd edition 2003).

Specifically, the vaccine comprises the vaccine antigen and/or a nucleic acid molecule encoding the vaccine antigen in a vaccine formulation, which preferably comprises an adjuvant.

A specifically preferred adjuvant is selected from the group consisting of alum (aluminum phosphate gel or aluminum hydroxide gel or mixture of the two), AS04 (alum plus monophosphoryl lipid A), MF59 (oil-in-water emulsion adjuvant), and toll-like receptor agonist adjuvants (monophosphoryl lipid A plus CpG).

The vaccine antigen described herein may be formulated with specific adjuvants commonly used in vaccines. For instance, a suitable selection of adjuvants may include MF59, aluminum hydroxide, aluminum phosphate, calcium phosphate, cytokines (e.g. IL-2, IL-12, GM-CSF), saponins (e.g. QS21), MDP derivatives, CpG oligonucleotides, LPS, MPL, polyphosphazenes, emulsions (e.g. Freund's, SAF), liposomes, virosomes, ISCOMs, cochleates, PLG microparticles, poloxamer particles, virus-like particles, heat-labile enterotoxin (LT), cholera toxin (CT), mutant toxins (e.g., LTK63 and LTR72), microparticles and/or polymerized liposomes. Suitable adjuvants are commercially available as, for example, AS01B (MPL and QS21in a liposome formulation), AS02A, AS15, AS-2, AS-03 and derivatives thereof (GlaxoSmithKline, USA); CWS (cell-wall skeleton), TDM (trehalose-6,6′-dimycolate), LeIF (Leishmania elongation initiation factor), aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7 or -12 may also be used as adjuvants. Preferred adjuvants for use in eliciting a predominantly Th1-type response include, for example, a combination of monophosphoryl lipid A, preferably 3-O-deacylated monophosphoryl lipid A (3D-MPL), optionally with an aluminum salt.

Another preferred adjuvant is a saponin or are saponin mimetics or derivatives, preferably QS21 (Aquila Biopharmaceuticals Inc.), which may be used alone or in combination with other adjuvants. For example, an enhanced system involves the combination of a monophosphoryl lipid A and saponin derivative, such as the combination of QS21 and 3D-MPL. Other preferred formulations comprise an oil-in-water emulsion and tocopherol. A particularly potent adjuvant formulation is QS21, 3D-MPL and tocopherol in an oil-in-water emulsion. Additional saponin adjuvants for use in the present invention include QS7 (described in WO 96/33739 and WO 96/11711) and QS17 (described in U.S. Pat. No. 5,057,540 and EP 0 362 279 B1).

The invention further provides for a vaccine comprising the vaccine antigen described herein in an effective amount, such as an immunogenic effective amount.

Specific embodiments of a vaccine comprise a nucleic acid molecule encoding the vaccine antigen. Specific examples of a vaccine are RNA-vaccines encoding the vaccine antigen. In particular, an RNA molecule can be used as a vaccine agent, in a naked form or formulated with a delivery vehicle. Specific embodiments may include a viral or bacterial host as gene delivery vehicle (e.g., live vaccine vector) or may include administering the gene in a free form, e.g., inserted into a plasmid. Specifically, the nucleic acid molecule encoding the vaccine antigen described herein is capable of expressing the folded RBD in a mammalian or human cell, and in particular upon vaccinating a subject.

Specifically, the vaccine comprises an effective amount of the vaccine antigen e.g., ranging between 0.001-1 mg per dose, preferably between 50 and 150 micrograms, such as about 100 micrograms.

The amount of vaccine antigen that may be combined with excipients to produce a single dosage form will vary depending upon the particular mode of administration. The dose of the vaccine antigen may vary according to factors such as age, sex and weight of the subject, and the ability to elicit the desired antibody response in the subject.

Dosage regime may be adjusted to provide the optimum therapeutic response. For example, several divided doses may be administered daily or the dose may be proportionally reduced as indicated by the requirements of the therapeutic situation. The dose of the vaccine may also be varied to provide optimum preventive dose response depending upon the circumstances.

Specifically, the vaccine described herein can be administered to the subject in an effective amount employing a prime-boost strategy.

For instance, the vaccine described herein may be administered to a subject several times according to a prime-boost regimen, at time intervals between the subsequent vaccinations ranging between 2 weeks and 5 years, preferably between 1 month and up to 3 years, more preferably between 2 months and 1.5 years. Specifically, the vaccine described herein is administered between 2 and 10, preferably between 2 and 7, even more preferably up to 5 and most preferably up to 3 times.

According to a specific embodiment, two or three doses are administered at time intervals of 3-4 weeks, to establish a protective immune response. The immune response can be boosted by administering one dose after 6 months following a first dose, and optionally every year. Booster administrations may serve to keep the antibody levels high.

The invention further provides a kit of components for preparing a vaccine described herein e.g., a pharmaceutical kit comprising one or more containers filled with one or more kit components, such as the vaccine antigen and an adjuvant. The kit can be used to prepare a vaccine in vitro, and/or upon administration. In a particular embodiment, the kit further comprises instructions for using the kit components.

The invention further provides for the vaccine for medical use.

According to a specific aspect, the invention further provides for the medical use of the vaccine antigen or vaccine described herein, or the nucleic acid molecule(s) encoding such antigen.

Specifically, the medical use involves an immunotherapy, such as an active immunotherapy. Specific immunotherapies provide for the treatment of a subject afflicted with, or at risk of contracting or suffering a disease or recurrence of a disease, by a method comprising inducing, enhancing, suppressing or otherwise modifying an immune response.

The invention further provides for a pharmaceutical preparation comprising the vaccine antigen described herein, further comprising a pharmaceutically acceptable carrier e.g., in an immunogenic formulation.

The invention further provides for the vaccine antigen or vaccine described herein for use in vaccinating a subject for prophylactic treatment against infection with a target virus, such as SARS-CoV-2, including naturally-occurring mutants thereof, preferably to elicit neutralizing antibodies recognizing the natural RBD.

The invention further provides for the vaccine for use in treating a subject to induce antibodies against SARS-CoV-2, and/or to produce an antiserum or a blood plasma product which comprises antibodies against SARS-CoV-2, preferably wherein said antibodies are SARS-CoV-2 neutralizing antibodies. Specifically, the blood plasma product is whole plasma (e.g., fresh frozen plasma), or a plasma fraction comprising antibodies e.g., IgG, and optionally IgA and/or IgM antibodies. Specifically, the blood plasma product is an immunoglobulin product or hyperimmunoglobulin product.

The invention further provides for the vaccine for use described herein, wherein the vaccine is administered to the subject by subcutaneous, intramuscular, intranasal, microneedle, mucosal, skin, or transdermal administration.

Therefore, the invention specifically provides for a method of treating a subject in need of prophylactic treatment by administering an effective amount of the vaccine e.g., to prevent a target virus infection, such as SARS-CoV-2 infection or the outbreak of a target virus disease, such as a SARS-CoV-2 disease or COVID-19.

According to the invention, there is further provided a method of preventing infectious disease in a subject, by vaccination and immunizing a subject in need thereof.

Specifically, the infectious disease is a disease or disease condition caused by a target virus.

Specifically, the target virus is SARS-CoV-2 (optionally including mutants of SARS-CoV-2). Where a heterologous protein is used that originates from another virus, such as HBV (e.g., HBV PreS polypeptide), the target virus is SARS-CoV-2 (optionally including mutants of SARS-CoV-2), and such other virus (e.g., HBV).

The invention further provides for a method for producing the vaccine antigen described herein, comprising expressing the vaccine antigen from the nucleic acid molecule described herein or an expression construct described herein. Specifically, expression of the nucleic acid molecule is in a recombinant eukaryotic expression system.

Specifically, a vaccine comprising the vaccine antigen is produced by combining the expressed vaccine antigen with any one or more of a pharmaceutically acceptable carrier, an excipient, or an adjuvant.

The invention further provides for a method for producing the vaccine or vaccine preparation described herein, by formulating the vaccine antigen described herein with any one or more of a pharmaceutically acceptable carrier, an excipient, or an adjuvant, such as to obtain a formulated vaccine preparation.

The invention further provides for a method of producing an RBD subunit vaccine with increased immunogenicity by fusing at least a first and a second folded RBD to said heterologous immunogenic carrier protein. Specifically, the vaccine antigen is an artificial fusion protein, wherein the heterologous immunogenic carrier protein is not naturally fused to an RBD in the S protein of SARS-CoV-2.

Specifically, said first and second folded RBDs are characterized by the features of said at least two RBDs of the vaccine antigen as described herein.

Specifically, the methods described herein refer to producing a vaccine antigen as further described herein.

Specifically, the vaccine antigen described herein is characterized by one or more of the following features:
a) the vaccine antigen comprises two, three or more RBDs;
b) said at least two RBDs consist of the same or different amino acid sequence;
c) at least one of said RBDs comprises or consists of an amino acid sequence of at least 180 amino acids length, and comprising at least 95% sequence identity to SEQ 20 ID NO:1 or 2, optionally comprising one or more point mutations which are the same as comprised in an RBD of one or more different naturally-occurring SARS-CoV-2 mutants;
d) said folded structure is
   i. obtained by expression of the vaccine antigen in a recombinant eukaryotic expression system, preferably employing mammalian, baculovirus-infected cells, or fungal host cells, preferably human host cells; and/or
   ii. determined by circular dichroism (CD) spectroscopy and/or an RBD-ACE2 interaction assay, preferably wherein the vaccine antigen is competing with a neutralizing anti-SARS-CoV-2 antibody preparation in the RBD-ACE2 interaction assay.
e) the vaccine antigen is provided as a single-chain fusion protein comprising said at least two RBDs fused to said heterologous immunogenic carrier protein, preferably comprising one or more peptide linker sequences;
f) the heterologous immunogenic carrier protein is a viral protein such as a surface protein or nucleocapsid protein, or a protein domain of any of the foregoing;
g) the heterologous immunogenic carrier protein is an antigen comprising B cell epitopes and T cell epitopes to elicit humoral and cellular immune responses in a human 5 subject,
h) the heterologous immunogenic carrier protein is a polypeptide that is not naturally fused to RBD;
i) the heterologous immunogenic carrier protein originates from any one of:
   i. a virus of the Hepadnaviridae family, such as a human hepatitis virus or hepatitis B virus, preferably wherein the heterologous protein is a surface protein of hepatitis B virus, such as a PreS or S protein; or
   ii. a beta-coronavirus, preferably any one of SARS-CoV-2, SARS-CoV, MERS, HCoV-OC43 or HKU1, preferably wherein the heterologous protein is selected from the group consisting of the S protein, or a subdomain thereof, such as an S1 or S2 domain, or a nucleocapsid (N) protein; or
   iii. a human rhinovirus serotype, preferably wherein the heterologous protein is a viral capsid protein such as any one of VP1, VP2, VP3, or VP4; or
   iv. a RSV, preferably wherein the heterologous protein is a G-protein, or central conserved region of the G-protein; or
   v. a glycolipid anchor, and wherein the RBDs fused to the anchor are surface-expressed by a virus-like particle comprising a lipid bilayer envelope and a core protein of an enveloped virus, such as Moloney murine leukemia virus (MoMLV), preferably wherein the core protein is MoMLV Gag and/or Gag-Pol; or
   vi. a naturally-occurring mutant of any one of the foregoing.
j) the heterologous immunogenic carrier protein is any other than an RBD of the spike (S) protein of SARS-CoV-2.
k) the heterologous immunogenic carrier protein is any one of:
   i. a Hepatitis B PreS polypeptide of at least 50% length of any one of SEQ ID NO:19-26, and comprising at least 80% sequence identity to the corresponding region of the respective SEQ ID NO:19-26, preferably wherein at least one RBD is fused to the N-terminus and at least one peptide is fused to the C-terminus of the PreS polypeptide; and/or
   ii. a glycosyl phosphatidylinositol (GPI)-anchor which is associated to the membrane of a virus-like particle expressed by a mammalian cell transfected with an expression plasmid encoding MoMLV gag-pol.

Specifically, the heterologous protein is characterized as further described herein, preferably any one of:
a) a Hepatitis B PreS polypeptide of at least 50% length, such as comprising at least 90% sequence identity to the corresponding region of any one of SEQ ID NO:19-26, preferably wherein at least one RBD is fused to the N-terminus and at least one peptide is fused to the C-terminus of the PreS polypeptide; and/or
b) a glycosyl phosphatidylinositol (GPI)-anchor which is associated to the membrane of a virus-like particle expressed by a mammalian cell transfected with an expression plasmid encoding MoMLV gag-pol.

### FIGURES

FIG. 1: IgG responses of convalescent COVID-19 patients and historic controls to microarrayed SARS-CoV-2 proteins. Protein-specific IgG levels (x-axes; proteins; y-axes, ISU in log₁₀ scale) in COVID-19 convalescent patients according to their virus neutralization titers (VNT) and in historic controls. p values <0.0001 for differences to historic controls are indicated as ***.

FIG. 2: Virus neutralization titers correlate with IgG levels to folded RBD and inhibition of RBD binding to ACE2. Correlation of virus neutralization titers (VNTs) in sera of COVID-19 convalescent subjects (x-axes, log 2 scale) with (A) levels of IgG antibodies (y-axis: ISU values) to folded RBD, and unfolded RBD or with (B) percentages of inhibition of RBD binding to ACE2 (y-axis: % inhibition).

FIG. 3: Patients' IgG antibodies recognize mainly conformational epitopes on folded RBD. Patients' IgG binding to folded or unfolded RBD without or with pre-adsorption with folded RBD, unfolded S1 or RBD peptide mix (x-axis). Y-axis: ISU values, log₁₀ scale, significant differences compared to no inhibition are indicated. p value: *** <0.0001.

FIG. 4: Characteristics of antibody responses of rabbits immunized with unfolded or folded RBD. IgG antibody levels (optical density OD levels, y-axes) of rabbits, three per group, immunized with 20 μg unfolded RBD to unfolded S1 (A), 40 μg unfolded RBD to unfolded S1 (B), 80 μg unfolded RBD to unfolded S1 (C), 20 μg unfolded RBD to folded RBD (D), 40 μg unfolded RBD to folded RBD (E), 80 μg unfolded RBD to folded RBD (F), 20 μg unfolded RBD to unfolded RBD (G), 40 μg unfolded RBD to unfolded RBD (H), 80 μg unfolded RBD to unfolded RBD (I), 20 μg unfolded RBD to HHM0 (J), 40 μg unfolded RBD to HHM0 (K), 80 μg unfolded RBD to HHM0 (L), 20 μg folded RBD to unfolded S1 (M), 40 μg folded RBD to unfolded S1 (N), 80 μg folded RBD to unfolded S1 (O), 20 μg folded RBD to folded RBD (P), 40 μg folded RBD to folded RBD (Q), 80 μg folded RBD to folded RBD (R), 20 μg folded RBD to unfolded RBD (S), 40 μg folded RBD to unfolded RBD (T), 80 μg folded RBD to unfolded RBD (U), 20 μg folded RBD to HHM0 (V), 40 μg folded RBD to HHM0 (W), 80 μg folded RBD to HHM0 (X). His-tagged control protein (HHM0). Time points of bleeding and serum dilutions are indicated in the insets. IgG binding (y-axes: ISU) of sera from rabbits (Y) immunized with 40 or 80 μg folded RBD, day 42 or, (Z) immunized with 40 or 80 μg unfolded RBD, day 42 after pre-adsorption with folded RBD, unfolded S1, peptide mix or with buffer alone (no inhibition) to microarrayed SARS-CoV-2 proteins and RBD-derived peptides.

FIG. 5: Shown are flow cytometric analyses of HEK293T cells transiently transfected with either pEAK12 FLAG::RBD::GPI (first row), pEAK12 FLAG::S::GPI (second row) or pEAK12 FLAG::NC::GPI (third row) stained with either anti-FLAG-PE antibody (first column), serum of a COVID-19 convalescent patient (second column) or serum of a healthy control subject (third column). Visualization of binding of human antibodies from serum samples (second and third column) was performed using a secondary antibody (goat-anti-human IgG Fab conjugated to APC).

FIG. 6: Shown are non-reducing immunoblot (IB) analyses of purified SARS-CoV-2 antigen-expressing VNP (10 μg/lane) expressing the indicated CD16-GPI-anchored virus antigens (FLAG::RBD::GPI from SARS-CoV-2, FLAG::S::GPI from SARS-CoV-2, FLAG::NC::GPI from SARS-CoV-2, FLAG::Art v 1::GPI from Artemisia vulgaris, Art v 1 from Artemisia vulgaris), control VNP (without antigen), rArt v 1 (2 μg) or rHis RBD from SARS-CoV-2 probed with serum from a COVID-19 convalescent patient (left column) or a healthy control subject (right column). Loading control was performed after stripping with an anti-MoMLVp30GAG monoclonal antibody (clone R187) against MoMLV capsid protein.

FIG. 7: Shown are reducing immunoblot (IB) analyses of purified antigen-expressing VNP (10 μg/lane) expressing the indicated GPI-anchored antigens (FLAG::RBD::GPI from SARS-CoV-2, FLAG::S::GPI from SARS-CoV-2, FLAG::NC::GPI from SARS-CoV-2, FLAG::Art v 1::GPI from Artemisia vulgaris, Art v 1 from Artemisia vulgaris), control VNP (without antigen), rArt v 1 (2 μg) or rHis RBD from SARS-CoV-2 probed with serum from a COVID-19 convalescent patient.

FIG. 8: Shown is the proliferation of PBMC from COVID-19 convalescent patients (black circles) and healthy control individuals (open circles) incubated with purified SARS-CoV-2 antigen-expressing VNP (5 μg/ml) expressing the indicated GPI anchored antigens (FLAG::RBD::GPI from SARS-CoV-2, FLAG::S::GPI from SARS-CoV-2, FLAG::NC::GPI from SARS-CoV-2, empty VNP (without antigen as a control), FSME antigen (0.15 μg/ml), Tetanus toxoid (0.0125 IE/ml), PHA (12.5 μg/ml) or medium alone for 144 h followed by a 18 hours methyl-[3H]-thymidine puls (1 μCi/well).

FIG. 9: Sequences referred to herein.

FIG. 10: (a): Structure of a fusion protein (PreS-RBD) consisting of two RBD domains, one fused to the N-terminus and one fused to the C-terminus of the human hepatitis virus B (HBV)-derived PreS, the HBV surface antigen containing the binding site of HBV to the NTCP (sodium taurocholate co-transporting polypeptide) receptor on hepatocytes; (b) Coomassie blue-stained SDS-PAGE containing E. coli- and HEK cell-expressed PreS-RBD and RBD separated under reducing and non-reducing conditions. Molecular weights are indicated in kDa; (c) Circular dichroism analysis of E. coli- and HEK cell-expressed PreS-RBD and RBD. Scans show molecular ellipticities (y-axes) at given wave lengths (x-axes). Reactivity of E. coli- and HEK cell-expressed PreS-RBD and RBD with different dilutions of (d) anti-His antibodies, (e) anti-PreS-peptide antibodies, (f) anti-recombinant PreS antibodies and (g) IgG antibodies (1:50 diluted) from COVID-19-convalescent subjects (n=10) and historic controls (n=10) by ELISA. OD (405/492 nm) values (y-axes) are average values of duplicate determinations with <5% deviation and correspond to amounts of bound antibodies. Buffer without primary antibodies served as negative control;

FIG. 11: RBD-specific IgG responses in rabbits immunized with different RBD-containing vaccines. Shown are IgG responses of rabbits, immunized with two equimolar RBD doses (20 or 40 microgram) of folded RBD monomer (RBD), folded RBD Dimer, folded RBD Trimer, folded PreS-RBD or folded N-RBD. IgG antibody levels specific for folded RBD (OD405/492 nm values) of three rabbits per group are shown for different time points of bleeding and serum dilutions as indicated. OD405/492 nm values are shown as means of duplicate determinations with <5% deviation, OD values 0.5 are considered positive and are indicated in bold.

FIG. 12: Immunization scheme of a healthy, SARS-Cov-2 negative subject indicating time points and dates of injection, sampling (serum, cells, mucosal fluids) during immunization with (a) unfolded E. coli- and (b) folded HEK cell-expressed PreS-RBD.

FIG. 13: Development of specific antibody responses in the immunized subject. Serum IgG reactivity to (a) folded RBD after immunization with unfolded E. coli (white stars) and folded HEK cell-expressed (black stars) PreS-RBD (x-axes, time points). (b) IgG reactivity to RBD mutations K417N, E484K, N501Y (alpha, B.1.1.7) and K417N+E484K+N501Y (beta, B.1.351) after immunization with HEK cell-expressed PreS-RBD at different time points (x-axis). (c) PreS-specific IgG after immunization with unfolded E. coli (white stars) and folded HEK cell-expressed (black stars) PreS-RBD (x-axes, time points). Sera were diluted 1:50, OD values are average values of duplicate determinations with <5% deviation (y-axes) and correspond to amounts of bound antibodies.

FIG. 14: SARS-CoV-2-specific protective antibodies in sera obtained at different time points from the subject, from COVID-19-convalescent patients and subjects after vaccination with registered SARS-CoV-2 vaccines.

FIG. 15. IgG responses to RBD (Wuhan) and RBD variants (delta, omicron) (inset) in (a) 1:50 diluted serum samples from the subject immunized with folded PreS-RBD at indicated time points or in (b) 1:1000 diluted sera from six rabbits (numbered 7-12) obtained three weeks after immunization with two doses (equimolar to 20 or 40 μg of RBD) folded PreS-RBD. OD values (y-axes) are averages of duplicate determinations with <5% deviation and correspond to bound antibodies.

### DETAILED DESCRIPTION OF THE INVENTION

Specific terms as used throughout the specification have the following meaning.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−10% or +/−5% of the given value.

The term “antigen” (herein also referred to as “immunogen”) as used herein, refers to any molecule that is recognized by the immune system and that can stimulate an immune response. In some embodiments, the antigen is a polypeptide or protein, and in particular a component of an infectious agent.

The term “antigen” as used herein shall in particular refer to any antigenic determinant, which can be possibly recognized by a binding site of an antibody or is able to bind to the peptide groove of HLA class I or class II molecules and as such may serve as stimulant for specific T cells. The antigen is either recognized as a whole molecule or as a fragment of such molecule, especially substructures e.g., a polypeptide or carbohydrate structure, generally referred to as “epitopes” e.g., B cell epitopes, T cell epitope), which are immunologically relevant i.e., are also recognizable by natural or monoclonal antibodies.

Specifically, preferred antigens are those molecules or structures, which have already been proven to be or are capable of being immunologically or therapeutically relevant, especially those, for which a clinical efficacy has been tested. The term as used herein shall in particular comprise molecules or structures selected from antigens comprising immuno-accessible and immuno-relevant epitopes, in particular conserved antigens found in one or more species or serotype. Immuno-accessible viral epitopes are typically presented by or comprised in antigens expressed on the outer surface of a virion or on the surface of an infected cell.

Selected epitopes and polypeptides as described herein may trigger an immune response in vivo, so to induce neutralizing antibodies against the antigen and target virus, respectively. This provides for the effective protection upon active immunization with the antigen. Polypeptide antigens are preferred antigens due to their inherent ability to elicit both cellular and humoral immune responses.

The term “epitope” as used herein shall in particular refer to a molecular structure which may completely make up a specific binding partner or be part of a specific binding partner to a binding site of an antibody. Chemically, an epitope recognized by antibodies may either be composed of a peptide, a carbohydrate, a fatty acid, an organic, biochemical or inorganic substance or derivatives thereof and any combinations thereof. If an epitope is a polypeptide, it will usually include at least 3 amino acids, preferably at least 4, 5, 6, 7, 8, 9, 10, 11, 12 or 13 amino acids. There is no critical upper limit to the length of the peptide, which could comprise nearly the full length of a polypeptide sequence of a protein. Epitopes can be either linear, sequential or discontinuous and if they assemble a structure can be conformational epitopes. A linear epitope is comprised of a single segment of a primary sequence of a polypeptide or carbohydrate chain. Discontinuous conformational epitopes are comprised of amino acids or carbohydrates brought together by folding of the polypeptide to form a tertiary structure and the amino acids are not necessarily adjacent to one another in the linear sequence. The vaccine antigens used herein specifically comprise one or more conformational epitopes that are comprised in a folded RBD, such as in natural RBD.

Immunogenicity of an antigen may be determined by suitable in vitro (such as ex vivo assays employing immune cells) or in vivo assays well-known in the art.

The immunogenicity of a vaccine antigen may be increased by combining the vaccine antigen with a heterologous element, such as fusing with an additional antigen or immunogen, or an immunogenic carrier. Specifically, the vaccine antigen described herein which comprises a heterologous RBD, or at least an RBD trimer, or which comprises a heterologous immunogenic carrier protein, such as a HBV PreS polypeptide, is found to have an increased immunogenicity as compared to the vaccine antigen without such heterologous element. For example, two copies of RBD fused to PreS induce higher levels or more consistently RBD-specific antibodies than two copies of RBD fused together without containing PreS upon immunization (see e.g., the examples, FIG. 11).

The vaccine antigen described herein specifically comprises RBDs of a certain conformation or fold, as produced by a eukaryotic expression system, or when expressed in a recombinant eukaryotic host cell.

The term “expression”, “expression cassette”, or “expression system” is herein understood as follows.

An expression cassette comprises at least a nucleic acid molecule (a polynucleotide), which contains a desired coding sequence to express the encoded polypeptide or protein of interest (POI), and control sequences in operable linkage, so that hosts (or host cells) transformed or transfected with these molecules incorporate the respective sequences and are capable of producing the respective encoded polypeptide or protein. An expression construct comprising an expression cassette may be comprised in an extrachromosomal vector, or be integrated into a host cell chromosome. Expression may refer to secreted or non-secreted expression products. The term “expression” as used herein refers to both, the expression of a polynucleotide or gene, or to the expression of the respective polypeptide or protein. The term “expressing a polynucleotide” or “expressing a nucleic acid molecule” as used herein, is meant to encompass at least one step selected from the group consisting of DNA transcription into mRNA, mRNA export, mRNA maturation, mRNA translation and processing, protein folding and/or protein transport.

A recombinant host organism comprises the expression cassette and means to express the polypeptide or protein of interest is herein understood as an “expression system”.

Expression cassettes are conveniently provided in a “vector” or “plasmid”, which are typically DNA sequences that are required for the transcription of cloned recombinant nucleotide sequences and the translation of their mRNA in a suitable host organism. Expression vectors or plasmids usually comprise an origin for autonomous replication or a locus for genome integration in the host cells, selectable markers (e.g., an amino acid synthesis gene or a gene conferring resistance to antibiotics such as zeocin, kanamycin, G418, hygromycin, or nourseothricin), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences, such as artificial chromosomes e.g., a yeast artificial chromosome (YAC).

Expression vectors may include but are not limited to cloning vectors, modified cloning vectors and specifically designed plasmids. Preferred expression vectors described herein are expression vectors suitable for expression of a recombinant gene in a eukaryotic host cell and are selected depending on the host organism. Appropriate expression vectors typically comprise regulatory sequences suitable for expressing DNA encoding a POI in a eukaryotic host cell. Examples of regulatory sequences include promoters, operators, enhancers, ribosomal binding sites, and sequences that control transcription and translation initiation and termination. The regulatory sequences are typically operably linked to the DNA sequence to be expressed.

To allow expression of a recombinant nucleotide sequence in a host cell, a promoter sequence is typically regulating and initiating transcription of the downstream nucleotide sequence, with which it is operably linked. An expression cassette or vector typically comprises a promoter nucleotide sequence which is adjacent to the 5′ end of a coding sequence, e.g., upstream from and adjacent to the coding sequence, or if a signal or leader sequence is used, upstream from and adjacent to said signal and leader sequence, respectively, to facilitate translation initiation and expression of coding sequences to obtain the expression product.

Specific expression constructs described herein comprise a polynucleotide encoding the POI linked with a leader sequence (e.g., a secretion signal peptide sequence (pre-sequence), or a pro-sequence), which causes transport of the POI into the secretory pathway and/or secretion of the POI from the host cell. The presence of such a secretion leader sequence in the expression vector is typically required when the POI intended for recombinant expression and secretion is a protein which is not naturally secreted and therefore lacks a natural secretion leader sequence, or its nucleotide sequence has been cloned without its natural secretion leader sequence. In general, any secretion leader sequence effective to cause secretion of the POI from the host cell may be used.

Expression systems, genetic constructs or modifications described herein may employ tools, methods and techniques known in the art, such as described by J. Sambrook et al., Molecular Cloning: A Laboratory Manual (3rd edition), Cold Spring Harbor Laboratory, Cold Spring Harbor Laboratory Press, New York (2001). Expression vectors may include but are not limited to cloning vectors, modified cloning vectors and specifically designed plasmids. Preferred expression vectors which may be used for the purpose of expressing sequences encoding a vaccine antigen described herein, are specifically expression vectors suitable for expression of a recombinant expression construct in a eukaryotic host cell and are selected depending on the host organism. Appropriate expression vectors typically comprise regulatory sequences suitable for expressing DNA encoding a recombinant protein in a eukaryotic host cell. Examples of regulatory sequences include promoters, operators, enhancers, ribosomal binding sites, and sequences that control transcription and translation initiation and termination. The regulatory sequences are typically operably linked to the DNA sequence to be expressed.

The term “folded” as used herein in the context of an RBD is herein understood as a folded secondary structure which confers functional binding of the RBD to its receptor hACE2, such as determined in an RBD-ACE2 interaction assay e.g., as described herein.

Specifically, the folded RBD structure is as occurring in natural RBD, or at least part of the fold as naturally occurring in natural RBD, such as including e.g., one alpha-helix and/or at least one beta-sheet fold and/or at least one disulfide bridge that is stabilizing the RBD-fold, which folded RBD structure provides for functionality of the RBD as determined in an RBD-ACE2 binding assay.

The folded RBD structure may or may not be stabilized by one or more disulfide bonds. The secondary structure may or may not comprise an alpha-helix and a β sheet structure, such as occurring in natural RBD, e.g., as determined by CD.

Functionality of the folded RBD may be determined by its binding to hACE2, such as determined in an RBD-hACE2 interaction assay, or in a BIACORE assay.

Reference is made to Lan et al. Nature 2020; 581:215-220; Wrapp et al. Science 2020; 367:1260-1263; and Wan et al. J. Virol. 2020; 94:e00127-20.

The term “host cell” as used herein shall refer to a single cell, a single cell clone, or a cell line of a host cell. The term “host cell” shall particularly apply to any cell, which is suitably used for recombination purposes to produce a protein of interest (“POI”) by an in vitro (or ex vivo) production method. It is well understood that the term “host cell” does not include human beings. The term “cell line” refers to an established clone of a particular cell type that has acquired the ability to proliferate over a prolonged period of time. A cell line is typically used for expressing a recombinant nucleic acid molecule. A “production host cell line” or “production cell line” is commonly understood to be a cell line ready-to-use for cell culture in a bioreactor to obtain the product of a production process, such as a POI.

Specific embodiments described herein refer to a recombinant production host cell line which is engineered to express the fusion protein as described herein, or the at least two RBDs comprised in the fusion protein. The RBD fold can depend on the type of the production host cell. For example, E. coli cells do not easily produce folded and functional RBD as shown herein, whereas mammalian cells produce folded and functional RBD also as shown herein.

Specifically, recombinant host cells as described herein are artificial organisms and derivatives of natural (understood as being naturally-occurring or wild-type) host cells. It is well understood that the host cells, methods and uses described herein, e.g., specifically referring to those comprising one or more genetic modifications, or artificial expression constructs, said transfected or transformed host cells and recombinant proteins, are non-naturally occurring, are “man-made” or synthetic, and are therefore not considered as a result of “law of nature”.

The term “heterologous” as used herein with respect to an amino acid sequence or protein, refers to a compound which is either foreign, i.e. “exogenous”, such as not found in nature e.g., in a natural (understood as being naturally-occurring or wild-type) protein; or that is found in a natural product, however, in the context of a heterologous construct (e.g., employing a heterologous nucleic acid sequence, or amino acid sequence), e.g. an artificial fusion of natural products or parts of natural products, which artificial fusions are not found in nature.

Specifically, the vaccine antigen described herein comprises a heterologous element which may be a protein (or protein domain) e.g., of at least any one of 100, 200, 300, 400, 500, 600, 700, 800, 900, or at least 1.000 amino acids length, or a polypeptide e.g., of at least 20, 30, 40, 50, 60, 70, 80, 90, or 100 amino acids length. Specifically, the heterologous element is part of a larger structure, such as a vaccine antigen, which element is exogenous, such that it is either foreign to the other parts of such larger structure as found in a natural protein, or not foreign to the other parts, but arranged in a non-natural way. Exemplary heterologous elements may be fused to the other parts, thereby obtaining fusion proteins that are not found in nature. Any recombinant or artificial nucleotide or amino acid sequence is understood to be heterologous. For example, a part (“element”) of a molecule that is not associated or fused with the other parts of the molecule as naturally-occurring or in a natural molecule (i.e., which is not naturally associated or fused) is understood as being heterologous. As an example, any artificial linker sequence as comprised in a recombinant fusion protein to link elements of such fusion protein, is a heterologous element of the fusion protein.

A specific exemplary heterologous element comprised in the vaccine antigen described herein is a viral polypeptide or protein originating from a virus other than SARS-CoV-2, such as a HBV PreS polypeptide.

Another specific exemplary heterologous element comprised in the vaccine antigen described herein is an additional RBD (a heterologous RBD) which originates from SARS-CoV-2 (or mutant thereof) or from any other beta-coronavirus, or which is an artificial mutant RBD that is obtained by mutagenesis to comprise one or more relevant point mutations as arising in one or more of a variety of SARS-CoV-2 mutants. Such heterologous RBD can be fused to another RBD comprised in the vaccine antigen, thereby obtaining at least an RBD-dimer or trimer.

The term “isolated” or “isolation” as used herein with respect to a polypeptide, protein or nucleic acid molecule such as the vaccine antigen and the nucleic acid molecule(s) encoding such vaccine antigen as described herein, shall refer to such compound that has been sufficiently separated from the environment with which it would naturally be associated, so as to exist in “purified” or “substantially pure” form. Yet, “isolated” does not necessarily mean the exclusion of artificial or synthetic fusions or mixtures with other compounds or materials, or the exclusion of impurities that do not interfere with the fundamental activity, and that may be present, for example, due to incomplete purification. Isolated compounds can be further formulated to produce preparations thereof, and still for practical purposes be isolated—for example, a set of peptides or the respective peptide fusions described herein can be mixed with pharmaceutically acceptable carriers, including those which are suitable for analytic, diagnostic, prophylactic or therapeutic applications, or excipients when used in diagnosis, medical treatment, or for analytical purposes.

The term “purified” as used herein shall refer to a preparation comprising at least 50% (w/w total protein), preferably at least 60%, 70%, 80%, 90% or 95% of a compound (e.g., a vaccine antigen described herein). A highly purified product is essentially free from contaminating proteins, and preferably has a purity of at least 70%, more preferred at least 80%, or at least 90%, or even at least 95%, up to 100%. Purity is measured by methods appropriate for the compound (e.g., chromatographic methods, polyacrylamide gel electrophoresis, HPLC analysis, and the like). An isolated, purified vaccine antigen described herein may be obtained as a recombinant product obtained by purifying from a host cell culture expressing the product in the cell culture supernatants, to reduce or remove host cell impurities or from cellular debris.

As isolation and purification methods for obtaining a purified polypeptide or protein product, methods utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used. The following standard methods can be used: cell (debris) separation and wash by Microfiltration or Tangential Flow Filter (TFF) or centrifugation, protein purification by precipitation or heat treatment, protein activation by enzymatic digest, protein purification by chromatography, such as ion exchange (IEX), hydrophobic interaction chromatography (HIC), Affinity chromatography, size exclusion (SEC) or HPLC chromatography, protein precipitation of concentration and washing by ultrafiltration steps. An isolated and purified protein can be identified by conventional methods such as Western blot, HPLC, activity assay, or ELISA.

With reference to nucleic acid molecules as described herein, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA (e.g., mRNA) molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated—for example, the polypeptides or polynucleotides can be mixed with pharmaceutically acceptable carriers or excipients when used in diagnosis or therapy.

The term “nucleic acid molecule” used herein refers to either DNA (including e.g., cDNA) or RNA (including e.g., mRNA) molecules comprising a polynucleotide sequence. The molecule may be a single or double-stranded polymer of deoxyribonucleotide or ribonucleotide bases read from the 5′ to the 3′ end. The term includes coding sequences, such as genes, artificial polynucleotides such as comprised in an expression construct expressing the respective polypeptide sequence. A DNA or RNA molecule can be used which comprises a nucleotide sequence that is degenerate to any of the sequences or a combination of degenerate sequences, or which comprises a codon-optimized sequence to improve expression in a host. For example, a specific eukaryotic host cell codon-optimized sequence can be used. Specific RNA molecules can be used to provide a respective RNA-vaccine.

A recombinant nucleic acid may be one that has a sequence that is not naturally occurring or that has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques well-known in the art. For example, a nucleic acid can be chemically synthesized using naturally-occurring nucleotides or variously modified nucleotides designed to increase the biological stability of the molecules or to increase the physical stability of the duplex formed upon hybridization.

Mutants of a naturally-occurring or natural protein or polypeptide as naturally-occurring in a wild-type source virus such as a SARS virus or a hepatitis virus, like RBD of SARS-CoV-2 or HBV PreS, as used herein may be provided e.g., by introducing a certain number of point mutations into a parent amino acid sequence. Specifically, a mutagenesis method is used to introduce one or more point mutations.

A point mutation as described herein is typically at least one of a deletion, insertion, and/or substitution of one or more nucleotides within a nucleotide sequence to achieve the deletion, insertion, and/or substitution of one (only a single one) amino acid at a certain, defined position within the amino acid sequence encoded by said nucleotide sequence. Therefore, the term “point mutation” as used herein shall refer to a mutation of a nucleotide sequence or an amino acid sequence. Specifically, preferred point mutations are substitutions, in particular conservative ones. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

Specific mutagenesis methods provide for point mutations of one or more nucleotides in a sequence, in some embodiments tandem point mutations, such as to change at least or up to 2, 3, 4, or 5 contiguous nucleotides within a nucleotide sequence of a parent molecule.

The term “mutagenesis” as used herein shall refer to a method of preparing or providing mutants of a nucleotide sequence and the respective protein encoded by said nucleotide sequence e.g., through insertion, deletion and/or substitution of one or more nucleotides, so to obtain variants thereof with at least one change in the coding region. Mutagenesis may be through random, semi-random or site directed mutation. A mutagenesis method can encompass methods of engineering the nucleic acid or de novo synthesizing a nucleotide sequence using the respective parent sequence information as a template.

Any of the exemplary proteins or polypeptides described herein may e.g., be used as a parent molecule and be modified to produce variants and mutants, which have substantially the same or an even improved immunogenic effect as the parent one, or which may include one or more point mutations which are also found in one or more different wild-type mutants of a virus. For instance, a library of nucleotide sequences may be prepared by mutagenesis of a selected parent nucleotide sequence encoding a protein or polypeptide originating from a wild-type source virus such as SARS-CoV-2 or HBV. A library of variants may be produced and a suitable mutant of the respective protein or polypeptide be selected according to a specifically desired genotype or phenotype.

As used herein, the term “mutant” with respect to a virus species or a viral protein, also referred to as “variant”, shall include all naturally-occurring or artificial compounds which differ from the respective original (parent) compound by at least one mutation that changes the structure or amino acid sequence of the parent compound. Mutants may differ in at least one amino acid that may change immunogenicity or the respective antibody response, such that antibodies induced by the parent compound no more recognize a mutant compound. To cover such mutants, it is preferred to mutagenize a parent vaccine antigen (or a part thereof e.g., at least one or at least two RBDs comprised in the vaccine antigen) such that all relevant point mutations that naturally-occur in one or more (a variety) of mutants be comprised in the mutagenized vaccine antigen, with the effect of inducing a protective immune response that covers not only the source virus of the parent vaccine antigen (or the part thereof originating from such source virus), but also the respective mutant virus(es) which are characterized by one or more of said relevant point mutations.

The term “naturally-occurring” as used herein with respect to a protein or polypeptide, or a specific point mutation, is understood to be found (occur) in a wild-type organism or virus (including wild-type mutant viruses). Mutants may be naturally-occurring or artificial. Naturally-occurring (also referred to as “wild-type”) proteins or polypeptides are herein also referred to as being “natural”. The present disclosure specifically refers to natural RBD, which is particularly understood as a molecule defined by a structure, fold and/or functionality, which is of RBD naturally-occurring in a SARS-CoV-2 virus (or SARS-virus), or in a naturally-occurring mutant thereof. Specifically, the secondary structure, fold and/or functionality of a natural RBD as described herein is found in, or corresponds to that of, or is essentially e.g., at least any one of 20%, 30%, 40%, 50%, 60%, 70%, 80%, or 90% the same or 100% the same as in a SARS-CoV-2 virus (or SARS-virus), or in a naturally-occurring mutant thereof. Specifically, natural RBD has a folded structure as in a pre- or postfusion conformation.

A point mutation is understood as a naturally-occurring point mutation if also comprised in a natural protein or polypeptide originating from a mutant virus.

Specifically, one or more of the RBDs of the vaccine antigen described herein may be natural RBD(s) originating from a source virus, or mutagenized to comprise one or more additional point mutations that are known to be comprised in any one or more mutants of the source virus. It is understood that not all point mutations comprised in a mutagenized RBD need to originate from the same mutant. One or more point mutations may originate from the same mutant, and one or more other point mutations may originate from another mutant.

The term “neutralizing” as used herein with respect to antibodies against a target virus is herein understood as follows. Specifically, neutralizing antibodies prevent SARS-CoV-2 from infecting the corresponding host cell. This may be achieved by inhibiting the binding of the virus to its receptor, ACE2 but also by inhibiting the fusion of the virus to the host cell membrane. Neutralizing SARS-CoV-2 antibodies can be tested by classical virus neutralization tests (VNTs) and also detected in the RBD-ACE2 interaction assay. SARS-CoV-2 neutralizing antibodies as described herein, due to their specific function, are expected to protect the host from getting infected with the virus.

Neutralizing activity against a virus strain can be tested in cell-based assays and in vivo. Neutralizing antibodies can be determined e.g., by enumerating virus titers in the presence of antibodies and detecting cytopathic effect in cell-based infection assays. One possible in vivo model for testing neutralizing activity against SARS-CoV2 is the Syrian Hamster model (Imai M. et al., Proc Natl Acad Sci USA. 2020; 117(28):16587-16595).

A “protective immune response” against a target virus is herein understood as follows. A protective immune response will protect the host from getting infected with the virus and/or will protect the host from developing severe COVID-19 disease.

Protective immune responses against SARS-CoV-2 can be measured using in vivo models of SARS-CoV-2 infection. When a vaccine antigen is designed to induce protective immunity, this may be tested e.g., by immunizing the animals with the vaccine antigen, and challenging the animals with SARS-CoV-2. Alternatively, the animal is immunized and then the antibodies are tested for their virus neutralization capacity as described herein.

The term “origin” or “originating” as used herein with respect to a naturally-occurring protein or polypeptide, or a virus species is herein understood to define a respective amino acid sequence which is identical to the respective naturally-occurring sequence, which is understood as a source, or which can be produced by modifying the naturally-occurring (source) sequence to produce a mutant or derivative thereof. Such mutant is herein understood as a mutant originating from the source.

According to a specific embodiment, the vaccine antigen described herein is produced as a recombinant polypeptide, such as produced by recombinant DNA technology.

As used herein, the term “recombinant” refers to a molecule or construct that does not naturally occur in a host cell. In some embodiments, recombinant nucleic acid molecules contain two or more naturally-occurring sequences that are linked together in a way that does not occur naturally. A recombinant protein refers to a protein that is encoded and/or expressed by a recombinant nucleic acid. In some embodiments, “recombinant cells” express genes that are not found in identical form within the natural (i.e., non-recombinant) form of the cell and/or express natural genes that are otherwise abnormally over-expressed, under-expressed, and/or not expressed at all due to deliberate human intervention. Recombinant cells contain at least one recombinant polynucleotide or polypeptide. “Recombination”, “recombining”, and generating a “recombined” nucleic acid generally encompass the assembly of at least two nucleic acid fragments.

The term “recombinant” as used herein specifically means “being prepared by or the result of genetic engineering” i.e., by human intervention. A recombinant nucleotide sequence may be engineered by introducing one or more point mutations in a parent nucleotide sequence, and may be expressed in a recombinant host cell that comprises an expression cassette including such recombinant nucleotide sequence. The polypeptide expressed by such expression cassette and host cell, respectively, is also referred to as being “recombinant”. For the purpose described herein conventional molecular biology, microbiology, and recombinant DNA techniques within the skill of the art may be employed. Specific embodiments described herein refer to the production of a vaccine antigen, and the recombinant means for such production, including a nucleic acid encoding the amino acid sequence, an expression cassette, a vector or plasmid comprising the nucleic acid encoding the amino acid sequence to be expressed, and a host cell comprising any such means. Suitable standard recombinant DNA techniques are known in the art and described inter alia in Sambrook et al., “Molecular Cloning: A Laboratory Manual” (1989), 2nd Edition (Cold Spring Harbor Laboratory press).

Methods for the production of fusion proteins are well known in the art and can be found in standard molecular biology references such as Sambrook et al. (Molecular Cloning, 2^{nd} ed., Cold Spring Harbor Laboratory Press, 1989) and Ausubel et al. (Short Protocols in Molecular Biology, 3^{rd} ed; Wiley and Sons, 1995). Typically, a fusion protein is produced by first constructing a fusion gene which is inserted into a suitable expression vector, which is, in turn, used to transfect a suitable host cell. Recombinant fusion constructs can be produced by a series of restriction enzyme digestions and ligation reactions which result in the desired sequences being incorporated into a plasmid, or by specific gene editing techniques. Synthetic oligonucleotide adapters or linkers can be used as is known by those skilled in the art and described in the references cited above. The elements of a fusion protein to be fused can be assembled prior to insertion into a suitable expression construct or vector. Insertion of the sequence within the vector should be in frame so that the sequence can be transcribed into a protein. The assembly of DNA constructs is routine in the art and can be readily accomplished by a person skilled in the art.

The term “sequence identity” of a variant or mutant as compared to a parent nucleotide or amino acid sequence indicates the degree of identity of two or more sequences. Two or more amino acid sequences may have the same residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 80%) sequence identity. Sequence similarity search tools frequently used are e.g., BLAST, FASTA, and HMMER.

The term “percent (%) amino acid sequence identity” as used herein with respect to an amino acid sequence, is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

For purposes described herein, the sequence identity between two amino acid sequences can be determined using the NCBI BLAST program version BLASTP 2.8.1 with the following exemplary parameters: Program: blastp, Word size: 6, Expect value: 10, Hitlist size: 100, Gapcosts: 11.1, Matrix: BLOSUM62, Filter string: F, Compositional adjustment: Conditional compositional score matrix adjustment.

For pairwise protein sequence alignment of two amino acid sequences along their entire length the EMBOSS Needle webserver (Pairwise Sequence Alignment; EMBL-EBI, Wellcome Genome Campus, Hinxton, Cambridgeshire, CB10 1SD UK,) was used with default settings (Matrix: EBLOSUM62; Gap open:10; Gap extend: 0.5; End Gap Penalty: false; End Gap Open: 10; End Gap Extend: 0.5). EMBOSS Needle uses the Needleman-Wunsch alignment algorithm to find the optimum alignment (including gaps) of the two input sequences and writes their optimal global sequence alignment to file.

The term “percent (%) identity” as used herein with respect to a nucleotide sequence, is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

For purposes described herein (unless indicated otherwise), the sequence identity between two amino acid sequences can be determined using the NCBI BLAST program version BLASTN 2.8.1 with the following exemplary parameters: Program: blastn, Word size: 11, Expect threshold: 10, Hitlist size: 100, Gap Costs: 5.2, Match/Mismatch Scores: 2,−3, Filter string: Low complexity regions, Mark for lookup table only.

The term “subunit vaccine” as used herein refers to a vaccine preparation that presents one or more antigens of a pathogen to the immune system without introducing the whole pathogen. A subunit vaccine may contain at least one antigen or immunogen, or which comprises at least two similar or dissimilar antigens or immunogens, that can elicit an immune response to a molecule or infectious antigen. Specifically, the vaccine antigen described herein is a subunit vaccine antigen comprising at least two folded RBDs as immunogens.

Herein the term “subject” is understood to comprise human or mammalian subjects, including livestock animals, companion animals, and laboratory animals, in particular human beings, which are either patients suffering from a specific disease condition or healthy subjects. In particular the treatment and medical use described herein applies to a subject in need of prophylaxis or therapy of a disease condition associated with a SARS-CoV-2 infection. Specifically, the treatment may be by interfering with the pathogenesis of a disease condition where SARS-CoV-2 is a causal agent of the condition. The subject may be a subject at risk of such disease condition or suffering from disease.

The term “at risk of” a certain disease conditions, refers to a subject that potentially develops such a disease condition e.g., by a certain predisposition, exposure to virus or virus-infected subjects, or that already suffers from such a disease condition at various stages, particularly associated with other causative disease conditions or else conditions or complications following as a consequence of viral infection.

The term “patient” includes human and other mammalian subjects that receive either prophylactic or therapeutic treatment. Subjects described herein may be patients or healthy subjects.

The term “treatment” as used herein shall always refer to treating subjects for prophylactic (i.e., to prevent infection and/or disease status) or therapeutic (i.e., to treat diseases regardless of their pathogenesis) purposes. The vaccine antigen described herein is specifically provided for active immunotherapy.

Specifically, the term “prophylaxis” refers to preventive measures which is intended to encompass prevention of the onset of pathogenesis or prophylactic measures to reduce the risk of pathogenesis.

The term “therapy” as used herein with respect to treating subjects refers to medical management of a subject with the intent to cure, ameliorate, stabilize, reduce the incidence or prevent a disease, pathological condition, or disorder, which individually or together are understood as “disease condition”.

The vaccine described herein specifically comprises the vaccine antigen in an effective amount, which is herein specifically understood as “immunologically effective amount”.

By “immunologically effective amount”, it is meant that the administration of that amount to a subject, either in a single dose or as part of a series of doses, is effective on the basis of the therapeutic or prophylactic treatment objectives. A “prophylactically effective amount” refers to an amount effective, at dosages and for periods of time necessary, to achieve the desired prophylactic result, such as preventing a target virus infection, or inhibiting a target virus disease onset or progression. This amount will vary depending upon the health and physical condition of the subject to be treated, age, the capacity of the subject's immune system to synthesize antibodies, the type and degree of immune response desired, the formulation of the vaccine, and other conditions.

An effective amount or dosage may range from 0.001 to 1 mg, e.g., between 0.05 and 0.15 mg, e.g., about 0.1 mg, of the vaccine antigen administered to the subject in need thereof, e.g., an adult human subject. For example, the effective dosage of the vaccine antigen is capable of eliciting an immune response in a subject of effective levels of antibody titer to bind and neutralize a target virus species e.g., 1-3 months after immunization. The effectiveness can be assayed by the respective antibody titers in samples of blood taken from the subject and in particular by measuring neutralizing antibodies. It may be also done by measuring virus-specific T cell responses.

In some embodiments, an effective amount is one that has been correlated with beneficial effect when administered as part of a particular dosing regimen e.g., a single administration or a series of administrations such as in a “boosting” regimen. For treatment, the vaccine described herein may be administered at once, or may be divided into the individual components and/or a number of smaller doses to be administered at intervals of time. Typically, upon priming a subject by a first injection of a vaccine according to the invention, one or more booster injections may be performed over a period of time by the same or different administration routes. Where multiple injections are used, subsequent injections may be made e.g., within 1 to 52 weeks of the previous injection.

The vaccine described herein may comprise the vaccine antigen in an immunogenic formulation. Specific embodiments comprise one or more adjuvants and/or pharmaceutically acceptable excipients or carriers.

Pharmaceutical carriers suitable for facilitating certain means of administration are well known in the art. Specific embodiments refer to immunogenic formulations, which comprise a pharmaceutically acceptable carrier and/or adjuvant, which trigger a humoral (B cell, antibody), helper or cytotoxic (T cell) immune response. Adjuvants may specifically be used to enhance the effectiveness of the vaccine. Adjuvants may be added directly to the vaccine compositions or can be administered separately, either concurrently with or shortly before or after administration of the vaccine antigen.

The term “adjuvant” as used herein specifically refers to a compound that when administered in conjunction with an antigen augments and/or redirects the immune response to the antigen, but when administered alone does not generate an immune response to the antigen. Adjuvants can augment an immune response by several mechanisms including lymphocyte recruitment, stimulation of B cells and/or T cells, and stimulation of macrophages and other antigen presenting cells, such as, e.g., dendritic cells.

An “effective amount” of an adjuvant can be used in a vaccine described herein, which is specifically understood to be an amount which enhances an immunological response to the immunogen such that, for example, lower or fewer doses of the immunogenic composition are required to generate a specific immune response and a respective effect of preventing or combating virus infection or disease.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Specific examples of pharmaceutically acceptable carriers include sterile water, saline, phosphate buffered saline, dextrose, glycerol, ethanol, polyethylene glycol, and the like, as well as combinations of any thereof. Additional pharmaceutically acceptable carriers are known in the art and described in, e.g., Remington: The Science and Practice of Pharmacy, 22^{nd} revised edition (Allen Jr, LV, ed., Pharmaceutical Press, 2012). Liquid formulations can be solutions, emulsions or suspensions and can include excipients such as suspending agents, solubilizers, surfactants, preservatives, and chelating agents. Exemplary carriers are liposomes or cationic peptides.

The preferred preparation is in a ready-to-use, storage stable form, with a shelf-life of at least one or two years. The invention also provides a delivery device e.g., a syringe, pre-filled with the vaccine according to the invention.

The vaccine described herein can be administered by conventional routes known within/to the vaccine field, such as via a parenteral (e.g., subcutaneous, intradermal, intramuscular, intravenous, or intraperitoneal) route, to a mucosal (e.g., ocular, intranasal, pulmonary, oral, gastric, intestinal, rectal, vaginal, or urinary tract) surface, or by topical administration to the skin (e.g., via a patch). The choice of administration route depends upon a number of parameters, such as the adjuvant used in the vaccine. If a mucosal adjuvant is used, the intranasal or oral route is preferred. If a lipid formulation or an aluminum compound is used, the parenteral route is preferred with the subcutaneous or intramuscular route being most preferred. The choice also depends upon the nature of the vaccine agent.

Therefore, the present invention provides for novel vaccine antigens, vaccines and methods of improving vaccine antigens to induce a neutralizing immune response, in particular neutralizing IgG antibodies conferring sterilizing immunity and protection against SARS-CoV-2.

The virus neutralization activity of infected patients' sera was found to be highly correlated with IgG antibodies specific for conformational but not sequential RBD epitopes and their ability to prevent RBD binding to its human receptor angiotensin-converting enzyme 2 (ACE2).

Only immunization with folded, but not with unfolded RBD, induced antibodies against conformational epitopes with high virus-neutralizing activity. Conformational RBD epitopes required for protection do not seem to be altered in the currently emerging virus variants, and are thus, most important to confer protection against such virus variants. These results are fundamental for estimating the protective activity of antibody responses after natural infection or vaccination and for the design of vaccines which can induce high levels of SARS-CoV-2-neutralizing antibodies conferring sterilizing immunity.

Herein described is the mapping of the polyclonal antibody responses in a large number of clinically well-characterized convalescent COVID-19 patients with a comprehensive panel of microarrayed folded and unfolded SARS-CoV-2 proteins and S-derived peptides in relation to their virus neutralization activity and ability to inhibit the RBD-ACE2 interaction. Experimental antibody responses induced by immunization with folded or unfolded RBD were analyzed for neutralization potential. A polyclonal antibody response against conformational RBD epitopes as in a folded RBD structure was found to be required for highly effective neutralization of SARS-CoV-2, and induction of this response was found to be possible upon immunization with folded RBD.

The foregoing description will be more fully understood with reference to the following examples. Such examples are, however, merely representative of methods of practicing one or more embodiments of the present invention and should not be read as limiting the scope of invention.

### EXAMPLES

### Example 1: Production of a Vaccine Antigen, Construct 1 (RBD-preS-RBD, SEQ ID NO:14) and Construct 3 (RBD-L-RBD-L-RBD, SEQ ID NO:16)

Expression of the fusion protein comprising folded RBD, in HEK cells Genes of interest in pcDNA3.1 were purchased from Genscript (Leiden, Netherlands) and codon-optimized for expression in HEK cells Vectors for expression in mammalian cells contain a CMV enhancer and promotor, IL-2 signal peptide, β-globin polyAterm and hygromicine resistance elements. For amplification of plasmid DNA in E. coli, the vector contains a pUC-Minimal-ORI. The plasmid was amplified in XL-21 E. coli.

Plasmid DNA was mixed with Expi Fectamine™ according to manufactures instructions and dropwise added to Expi293 HEK cells (Thermo Fisher Scientific) (4×10⁶ cells/ml). Cells were incubated in a 37° C. incubator with ≥80% relative humidity and 8% CO₂ on an orbital shaker platform for 4-6 days. Cells were harvested by centrifugation and Ni²⁺⁺-affinity purification was performed as described (Gattinger et al. EBioMedicine. 2019; 39:33-43).

Vaccine antigens comprising folded RBD were produced by fusing an RBD dimer to a heterologous element. Exemplary vaccine antigens: Construct 1 (RBD-PreS-RBD, SEQ ID NO:14) and Construct 3 (RBD-L-RBD-L-RBD, SEQ ID NO:16).

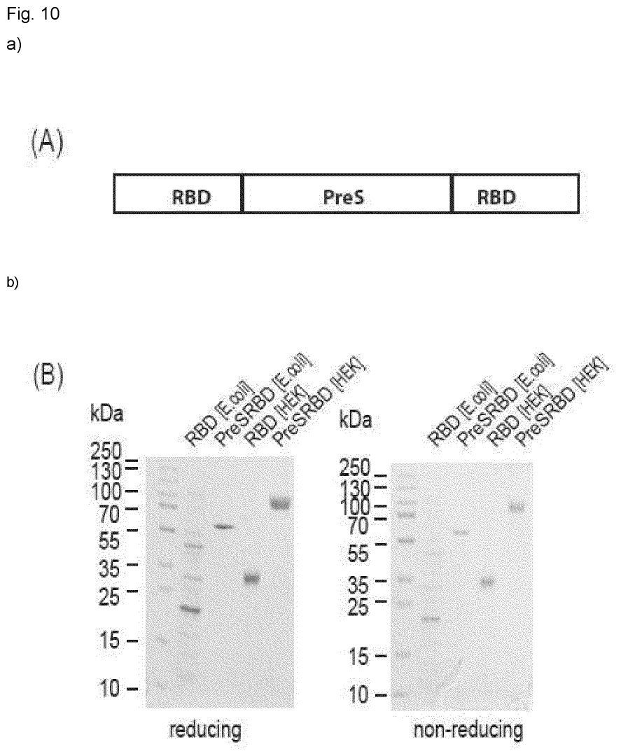

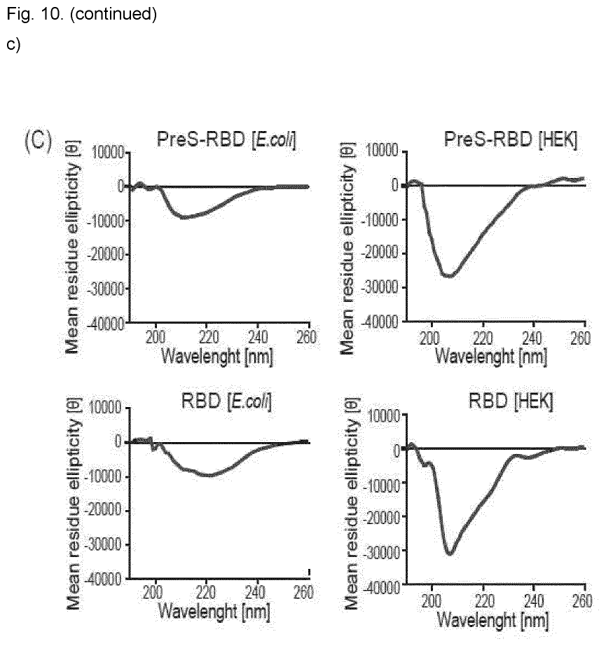

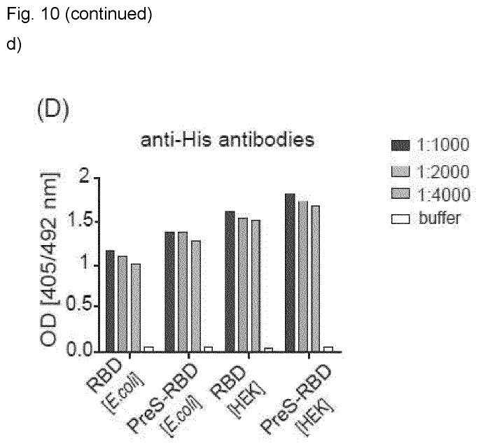

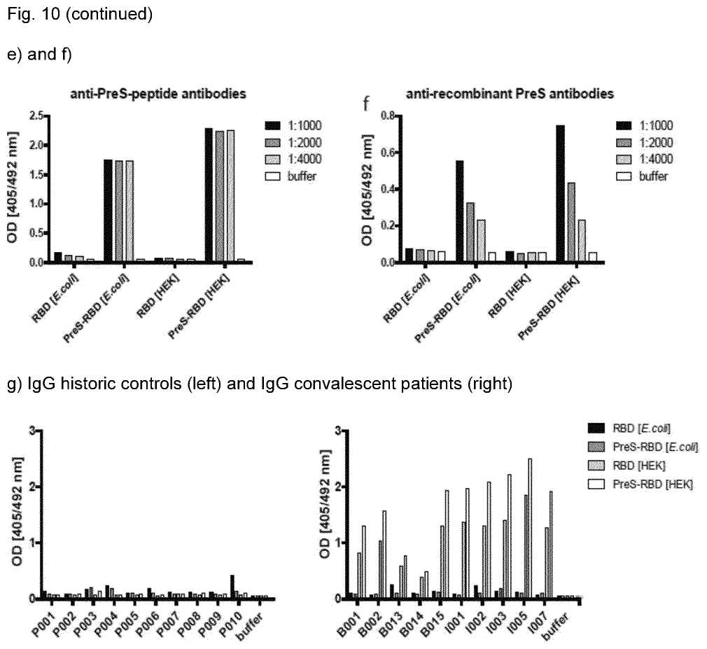

According to the present example, the PreS protein of the HBV surface antigen containing the binding site of HBV to the NTCP (sodium taurocholate co-transporting polypeptide) receptor on hepatocytes domain was used as an immunogenic carrier protein (FIG. 10a).

Expression of the Fusion Protein Comprising Unfolded RBD, in E. coli

Synthetic genes were cloned into the NdeI and XhoI site of plasmid pET27b, transformed into E. coli BL21-DE3 (Agilent Technologies, Santa Clara, CA, USA). Expression of recombinant proteins was induced in liquid LB cultures containing kanamycin with 1 mM IPTG (Roth, Karlsruhe, Germany) after an OD600 of 0.5 was achieved. E. coli cells were harvested after 2.5 hours by centrifugation and lysis of the pellet was performed with 6M GuHcl pH 6.3 for 2 hours at 4° C. After centrifugation the supernatant was incubated with Ni-NTA Agarose (Qiagen, Hilden, Germany) for 4 hours, washed with 50-fold bed volume 100 mM NaH₂PO₄, 10 mM Tris, 8 M Urea pH 6.4 and eluted with 100 mM NaH₂PO₄, 10 mM Tris, 20 mM Hepes, 8 M Urea, pH 4.5. Then a stepwise dialysis to 20 mM NaH₂PO₄, 10 mM Tris, 20 mM Hepes, pH 4.5 was performed.

### Example 2: Determining the RBD Fold and Functionality in a SARS-CoV-2-ACE2 Interaction Assay

In order to evaluate whether RBD is functional and binds to its receptor ACE2, a molecular interaction assay mimicking SARS-CoV-2 binding to its receptor ACE2 can be used. This ELISA assay is based on plate-bound recombinant ACE2 which, for example, is allowed to bind to recombinant His-tagged RBD as described (Gattinger P. et al. Allergy. 2021; 76(3):878-883). Bound RBD is then detected with a mouse monoclonal anti-His antibody followed by a secondary HRP-labelled anti-mouse IgG₁ antibody.

Using this assay, specific binding of RBD to ACE2 occurs in a dose-dependent and specific manner whereas a negative control protein, the cysteine-containing, His-tagged recombinant Parietaria allergen, Par j 2, did not bind to ACE2. The optical density levels reflecting binding which are obtained with the negative control protein plus 3-fold the standard deviation are subtracted from the optical density measured for RBD to determine whether RBD can bind specifically. For additional control, binding of RBD to ACE2 can be blocked specifically by pre-incubation with soluble ACE2 (Gattinger P. et al. Allergy. 2021; 76(3):878-883). This control experiment is further controlled by showing that pre-incubation with a negative control protein instead of ACE2, for example recombinant major birch pollen allergen, Bet v 1, does not affect binding of RBD to ACE2 (Gattinger P. et al. Allergy. 2021; 76(3):878-883).

According to this specific example, the interaction assay was performed, in brief: human ACE2 protein (GenScript) was coated (2 μg/ml) in bicarbonate buffer overnight onto NUNC Maxisorb 96 well plates (Thermofisher). Plates were washed 3 times with washing buffer and subsequently blocked for 3 hours at RT with blocking buffer. Meanwhile serum samples were diluted 1:2 in PBS, 0.05% Tween 20, 1% BSA and incubated for 2 hours with 200 ng His-tagged RBD (GenScript). For control purposes, 10 μg/ml ACE2 protein (positive control) and 10 μg/ml Bet v 1 (negative control) were pre-incubated with 100 ng His-tagged RBD.

The overlay was performed by adding the pre-incubated RBD samples to the coated and blocked ACE2 protein followed by incubation for 3 hours. The plates were washed and incubated overnight with 1:1000 diluted mouse anti-His tag antibody (Dianova, Hamburg, Germany). After 3 washing three times, 1:1000 diluted HRP-linked 10 anti-mouse IgG₁ antibody (GE Healthcare) was incubated 2 hours and detected by ABTS. The mean optical density (O.D.) values corresponding to the amount of bound RBD were measured at 405 nm and 492 nm (reference) in a TECAN Infinite F5 ELISA reader with the integrated software i-control 2.0 (Tecan Group Ltd., Männedorf, Switzerland). ACE2 protein and Bet v 1 served as positive and negative controls in the blocking experiments, respectively. From each measurement the buffer control (overlay without RBD) was subtracted. All determinations were performed in duplicates and results are shown as mean values with a variation of <5%. The percentages of inhibition were calculated as follows:

Percentage inhibition (%)=(OD_{Betv 1}−OD_{Serum})/(OD_{Betv 1}−OD_{ACE2})×100

### Example 3: Determining the RBD Fold by Far-UV Circular Dichroism (CD) Spectroscopy

The far-UV (ultraviolet) CD spectrum of proteins can reveal important characteristics of their secondary structure. CD spectra can be readily used to estimate the fraction of a molecule that is in the alpha-helix conformation, the beta-sheet conformation, the beta-turn conformation, or some other (e.g., random coil) conformation. CD is a standard technique and a valuable tool, especially for showing changes in conformation. It can, for instance, be used to study how the secondary structure of a molecule changes as a function of temperature or of the concentration of denaturing agents, e.g., Guanidinium chloride or urea. Thus, CD is a valuable tool for verifying that the protein is in its native conformation before undertaking extensive and/or expensive experiments with it.

CD gives less specific structural information than X-ray crystallography and protein NMR spectroscopy, for example, which both give atomic resolution data. However, CD spectroscopy is a quick method that does not require large amounts of proteins or extensive data processing. Thus, CD can be used to survey a large number of solvent conditions, varying temperature, pH, salinity, and the presence of various cofactors.

Typically, the native RBD fold is determined by far UV-CD spectroscopy, if at least 20% of the protein is shown in a folded conformation, preferably at least any one of 20%, 30%, 40%, 50%, 60%, 70%, 80%, 85%, 90%, 95%, 98%, 99%, or 100% of the protein is present in a folded conformation as shown in a far-UV CD spectrum.

According to this specific example, circular dichroism spectroscopy was performed, in brief: far UV circular dichroism (CD) spectra of recombinant proteins were collected on a Jasco J-810 spectropolarimeter (Japan Spectroscopic Co., Tokyo, Japan) using a 1 mm path length quartz cuvette at protein concentrations of 0.1 and 0.26 mg/mL, respectively. Spectra were measured from 260 to 180 nm, with a 0.5 nm resolution at a scanning speed of 50 nm/min, and resulted from averaging of three scans. All measurements were performed in 10 mM Na₂HPO₄ pH 7. The final spectra were baseline corrected by subtracting the corresponding buffer spectrum. Results were expressed as the mean residue ellipticity [θ] at a given wavelength. The secondary structure content of recombinant was calculated using the secondary structure estimation program CDSSTR.

### Example 4: Immunization with Folded but not Unfolded RBD Induces Virus-Neutralizing Antibodies

Folded RBD expressed in HEK293 cells was adsorbed onto aluminum hydroxide (SERVA Electrophoresis, Heidelberg, Germany) resulting in three dose formulations containing, 20 μg, 40 μg and 80 μg protein per 0.75 mg aluminum hydroxide in 0.5 ml 50 mM NaH₂PO₄, 10 mM Tris, 20 mM, HEPES, 0.9% NaCl, pH 4.5 per protein, respectively. Likewise, a fusion protein consisting of the unfolded receptor-binding domain (RBD) with HBV-derived PreS (PreS-RBD) was adsorbed to aluminium hydroxide. For control purposes, also a mix without protein containing 0.75 mg Aluminum hydroxide in 0.5 ml 50 mM NaH₂PO₄, 10 mM Tris, 20 mM, HEPES, 0.9% NaCl, pH 4.5 was prepared. Three rabbits per protein dose or control formulation were immunized subcutaneously 4 times in a three-weekly interval (Charles River, Chatillon sur Chalaronne, France). Serum samples from rabbits were obtained before the first immunization (pre-immune sera) and on days 21, 28, 35, 42 and 64 after the first immunization. Sera were stored at −20° C. until use.

### Example 5: Neutralization of SARS-CoV-2 Requires Antibodies Against Conformational Receptor-Binding Domain Epitopes

This example illustrates that neutralization of SARS-CoV-2 requires antibodies against conformational receptor-binding domain (RBD) epitopes and these antibodies can be induced only by vaccination with folded RBD.

The determinants of a successful humoral immune response to the severe acute respiratory syndrome coronavirus 2 (SARS-CoV-2) are of critical importance for the design of effective vaccines and the evaluation of the degree of protective immunity conferred by exposure to the virus. As novel variants emerge, understanding their likelihood of suppression by population antibody repertoires has become increasingly important. In this study the SARS-CoV-2 polyclonal antibody response was analyzed in a large population of clinically well-characterized patients after mild and severe COVID-19 using a panel of microarrayed structurally folded and unfolded SARS-CoV-2 proteins, as well as sequential peptides, spanning the surface spike protein S and the receptor-binding domain (RBD) of the virus. The S- and RBD-specific antibody response was dominated by IgG, mainly IgG₁, and directed against structurally folded S and RBD and three distinct peptide epitopes in S2. The virus-neutralization activity of patients' sera was highly correlated with IgG antibodies specific for conformational but not sequential RBD epitopes and their ability to prevent RBD binding to its human receptor angiotensin-converting enzyme 2 (ACE2). Twenty percent of patients selectively lacked RBD-specific IgG. Only immunization with folded, but not with unfolded RBD, induced antibodies against conformational epitopes with high virus-neutralizing activity. Conformational RBD epitopes required for protection do not seem to be altered in the currently emerging virus variants. These results are fundamental for estimating the protective activity of antibody responses after natural infection or vaccination and for the design of vaccines, which can induce high levels of SARS-CoV-2-neutralizing antibodies conferring sterilizing immunity.

### Recombinant and Natural Proteins, Synthetic Peptides

Synthetic genes (SARS-CoV-2 Genbank accession Nr.: QHD43416.1) encoding the receptor-binding subunit (S1), the membrane fusion subunit (S2) and a fusion protein consisting of the receptor-binding domain (RBD) with HBV-derived PreS (PreS-RBD) (35) each of them containing a DNA encoding a C-terminal hexahistidine tag and codon-optimized for bacterial expression were obtained from ATG:biosynthetics (Merzhausen, Germany) (Table 1). Synthetic genes were cloned into the Nde I and Xho I site of plasmid pET27b, transformed into E. coli BL21-DE3 (Agilent Technologies, Santa Clara, CA, USA). Expression of recombinant proteins was induced in liquid LB cultures containing kanamycin with 1 mM IPTG (Roth, Karlsruhe, Germany) after an OD600 of 0.5 was achieved. E. coli cells were harvested after 2.5 hours by centrifugation and lysis of the pellet was performed with 6M GuHcl pH 6.3 for 2 hours at 4° C. After centrifugation the supernatant was incubated with Ni-NTA Agarose (Qiagen, Hilden, Germany) for 4 hours, washed with 50-fold bed volume 100 mM NaH₂PO₄, 10 mM Tris, 8 M Urea pH 6.4 and eluted with 100 mM NaH₂PO₄, 10 mM Tris, 20 mM Hepes, 8 M Urea, pH 4.5. Then a stepwise dialysis to 20 mM NaH₂PO₄, 10 mM Tris, 20 mM Hepes, pH 4.5 was performed. Protein concentrations were measured with Micro BCA Protein Assay Kit (Pierce, Rockford, Illinois, USA). The expression and purification of His-tagged control proteins, non-glycosylated and glycosylated horse heart myoglobin (HHM0, HHM2), was performed as described (36). Purified recombinant SARS-CoV-2 proteins expressed in E. coli or eukaryotic systems which had been purchased are listed in Table 1. The analysis of the secondary structure of the aforementioned proteins by circular dichroism analysis was performed as previously described (36). Natural and recombinant control proteins used in the microarray and their origin are listed in Table 3.

Overlapping 25-30mer peptides covering the amino acid sequence of SARS-CoV-2 spike protein (Genbank accession Nr.: QHD43416.1) (Table 2) were synthesized by solid-phase synthesis using 9-fluorenyl-methoxy carbonyl (Fmoc)-method on a microwave synthesizer Liberty blue (CEM-Liberty, Matthews, NC, USA and Applied Biosystems, Carlsbad, CA, USA) on Wang preloaded resins (Merck, Darmstadt, Germany) as previously described (19,37). Thereafter, resins were washed with 50 ml dichloromethane (Roth, Karlsruhe, Germany) and peptides were cleaved from the resins by adding 28.5 ml trifluoroacetic acid (Roth, Karlsruhe, Germany), 0.75 ml silane (Sigma-Aldrich, St. Louis, MO, USA) and 0.575 ml H₂O and incubating for 2.5 hours at RT. After precipitation in pre-cooled tert-butylmethylether (Merck, Darmstadt, Germany), purification by reverse-phase HPLC using a Aeris 5 μm peptide 250×21.2 mm column (Phenomenex, Torrance, CA, USA) and molecular weight identification by mass spectrometry (Microflex MALDI-TOF, Bruker, Billerica, MA, USA) was performed as described (19,37). The solvent accessible surface areas of peptides 13 to 21 (Table 2) were calculated in PyMOL (PyMOL Molecular Graphics System, Version 2.5.0a0 Schrödinger, LLC) using PDB entry 6XR8. A probe radius of 1.4 Å was used and the results are given in Å², as well as percentage of the theoretical solvent accessible area obtained when the peptide without the surrounding spike protein is used for the calculation.

### Immunization of Rabbits

Unfolded PreS-RBD expressed in E. coli or folded RBD expressed in HEK293 cells were adsorbed onto aluminum hydroxide (SERVA Electrophoresis, Heidelberg, Germany) resulting in three dose formulations containing, 20 μg, 40 μg and 80 μg protein per 0.75 mg aluminum hydroxide in 0.5 ml 50 mM NaH₂PO₄, 10 mM Tris, 20 mM, HEPES, 0.9% NaCl, pH 4.5 per protein, respectively. For control purposes, also a mix without protein containing 0.75 mg Aluminum hydroxide in 0.5 ml 50 mM NaH₂PO₄, 10 mM Tris, 20 mM, HEPES, 0.9% NaCl, pH 4.5 was prepared. Three rabbits per protein dose or control formulation were immunized subcutaneously 4 times in a three-weekly interval (Charles River, Chatillon sur Chalaronne, France). Serum samples from rabbits were obtained before the first immunization (pre-immune sera) and on days 21, 28, 35, 42 and 64 after the first immunization. Sera were stored at −20° C. until use.

### Detection of Specific Antibody Responses by ELISA

Immunoglobulin response of human serum samples of COVID-19 convalescent patients and healthy control sera to SARS-CoV-2 derived proteins was determined by ELISA as previously described (25) with the following alterations: Aliquots of 2 μg/ml of S or folded RBD (Genscript, Leiden, Netherlands) were coated overnight onto NUNC Maxisorp 96 well plates (Thermofisher, Waltham, MA, USA). After washing the plates 3 times with wash buffer (PBS, 0.05% Tween 20) and blocking at RT for 3 hours, serum samples were diluted 1:40 and incubated overnight. Plates were washed 3 times and incubated for 2 hours with HRP-conjugated anti-human IgG (BD, San Jose, CA, USA) diluted 1:1000, washed 3 times and developed with ABTS (Sigma-Aldrich, St. Louis, MO, USA). Bound human IgM, IgA and IgG₁₋₄ antibodies were measured as described (38). The optical density was measured at 405/492 nm with Infinite F50 ELISA reader after 10 minutes (Tecan, Männedorf, Switzerland).

Rabbit IgG antibody responses to insect cell-expressed folded S, HEK cell-expressed folded RBD (both Genscript, Leiden, Netherlands) and unfolded S1 expressed in E. coli as well as to a non-glycosylated His-tagged control protein HHM0 was measured by ELISA. Aliquots of 2 μg/ml of each of the proteins were coated overnight, plates were blocked for at RT 3 hours and incubated with rabbit sera in two-fold dilutions overnight. Bound rabbit IgG was detected by incubation with donkey anti-rabbit horseradish peroxidase-coupled IgG antibodies diluted 1:1000 (GE Healthcare UK Limited, Chalfont St Giles, United Kingdom) for 2 hours and subsequent ABTS development as described above.

All measurements were performed in duplicates with a variation of <5% for means. Background threshold levels (i.e., means of the corresponding buffer control plus three times standard deviation thereof) for each protein and immunoglobulin class or subclass were subtracted.

### SARS-CoV-2 Microarray

Glass slides containing six microarrays surrounded by an Epoxy frame (Paul Marienfeld GmbH & Co. KG, Lauda-Königshofen, Germany) were coated with an amine-reactive complex organic polymer, MCP-2 (Lucidant Polymers, Sunnyvale, CA, USA). Spotting conditions were optimized for each protein/peptide to obtain round-shaped compact spots of comparable size. For the final microarray printing, SARS-CoV-2 antigens were spotted at the concentration of 0.5-1 mg/ml in phosphate buffer (75 mM Na₂HPO₄, pH=8.4) in triplicates using a SciFlexArrayer S12 (Scienion AG, Berlin, Germany) (19). IgG, IgM and IgA reactivity to micro-arrayed proteins and peptides in sera was measured as follows: Microarrays were washed for 5 min with PBST and dried by centrifugation. Subsequently, 35 μl of a 1:40 diluted serum sample (sample diluent, Thermofisher, Waltham, MA, USA) was added per array und incubated for 2 hours. After another washing step, 30 μl of secondary antibodies were applied and incubated for 30 min at RT. Secondary antibodies were DyLight 550 (Pierce, Rockford, IL, USA) labeled anti-human IgG (Jackson ImmunoResearch Laboratories, West Grove, PA, USA), anti-human IgM or anti-human IgA (both BD, San Jose, CA, USA) at a final concentration of 1 μg/ml, respectively. Slides were again washed, dried and subsequently scanned using a confocal laser scanner (Tecan, Männedorf, Switzerland). Image analysis was performed by MAPIX microarray image acquisition and analysis software (Innopsys, Carbonne, France) and conversion of measured fluorescence units to ISAC standardized units (ISU) was performed as described (Ref. 19,37).

For micro-arrayed inhibition experiments, human sera were diluted 1:100, rabbit sera 1:800 in sample diluent and pre-incubated overnight with either folded RBD, unfolded RBD, unfolded S1 (10 μg/ml) or with an equimolar amount of a RBD-derived peptide mix comprising peptides 13-21 (Table 2), respectively. For the detection of bound rabbit IgG, DyLight 550 labeled anti-rabbit IgG antibody (Thermofisher, Waltham, MA, USA) was used at a final concentration of 1 μg/ml. Micro-arrayed measurements and analysis were performed as described as above.

### Determination of SARS-CoV-2 VNTs and Inhibition of the RBD-ACE2 Interaction

The molecular interaction assay to detect inhibition of RBD to ACE2 receptor binding by patients' sera was performed as described (Ref. 25). Shortly, 1:2 diluted serum was incubated for 3 hours with HEK cell-expressed His-tagged RBD followed by a 3-hours overlay onto plate bound ACE2. Bound RBD was then detected with a mouse monoclonal anti-His antibody followed by a HRP-labelled anti-mouse IgG₁ antibody and detected with ABTS. All measurements were performed in duplicates with a variation of <5%. The SARS-CoV-2 neutralization test (VNT) was carried out as described (39). Two-fold serial dilutions of heat-inactivated serum samples were incubated with 50-100 TCID50 SARS-CoV-2 for 1 hour at 37° C. The mixture was added to Vero E6 cell (ATCC® CRL-1586) monolayers and incubation was continued for three days at 37° C. VNTs were expressed as the reciprocal of the serum dilution that protected against virus-induced cytopathic effects. VNT titers >10 were considered positive.

### Visualization of RBD Peptides and Reported RBD Mutations in the Spike Protein Structure

Surface representation of SARS-CoV-2 spike protein was generated in PyMOL (PyMOL Molecular Graphics System, Version 2.5.0a0, Schrödinger, LLC) based on the PDB entry 6XR8. Currently known mutations in RBD were derived from https://spikemutants.exscalate4cov.eu/.

### Results

### Overview of the Study Population

From 29^{th} of April 2020 to 30^{th} of July 2020, 253 COVID-19 convalescent patients with positive SARS-CoV-2 RT-PCR test and/or positive antibody tests and 235 age and gender-matched control subjects who had no signs of COVID-19 or common-cold-like symptoms were enrolled in this study. The COVID-19 patient group consisted of 139 patients (54.9%) who had mild symptoms (myalgia and anosmia: 59.7%; cough: 68.3%; fever: 73.4%) which were treated at home without requiring hospitalization and 114 patients (45.1%) with severe symptoms who had been hospitalized and had received oxygen or were treated by intensive care. Mild COVID-19 patients did not have pneumonia whereas 65.8% of the severe patients had pneumonia. Characteristics (i.e., symptoms, comorbidities) of COVID-19 patients were similar to those reported in other studies (17). Patients with severe symptoms showed a significantly higher prevalence of cardiopulmonary and endocrine co-morbidities, in particular diabetes and hypertension, compared to patients with mild COVID-19. Fatigue, myalgia and anosmia were significantly more frequent in the mild group (59.7%) than in the severe group (42.2%). The percentages of patients suffering from IgE-associated allergy were similar among patients with mild and severe COVID-19 and the control group. The 235 control subjects had a negative SARS-CoV-2 RT-PCR test at the time of investigation and no common cold-like symptoms in the 10 weeks before the visit. Overall, the prevalence of malignancies, endocrine or circulatory co-morbidities were significantly higher in COVID-19 patients as compared to control individuals. Blood samples were collected from COVID-19 convalescent patients approximately 8 weeks (mean 61 days, SD±13.7, min. 19 days, max. 98 days) after the positive SARS-CoV-2 RT-PCR test, which ensured that they had seroconverted and were already in the plateau-phase of antibody production (Ref. 8,18). To discriminate between antibodies specific for SARS-CoV-2 and those acquired through earlier infections by common cold-inducing coronaviruses, sera obtained before the occurrence of COVID-19 (i.e., 1996-summer 2019, historic controls) from 38 age-matched control subjects were included in the analyses.

### Microarray of Folded and Unfolded SARS-CoV-2 Proteins and S-Derived Peptides

In order to study the polyclonal antibody response of COVID-19 patients against a comprehensive set of antigens simultaneously for each serum, a microarray containing a panel of SARS-CoV-2-derived antigens, S-derived peptides and control antigens in triplicates was created (Tables 1-3). The antigens had been expressed in eukaryotic expression systems or Escherichia coli and according to circular dichroism (CD) analysis represented folded or unfolded proteins (Table 1). S-derived peptides of approximately 30 amino acids in length spanning the S-protein and in particular RBD were included (Table 2). The analysis of the surface exposure of RBD-derived peptides indicated that the percentages of surface-exposed amino acids were highest for peptides 13-15 and 18-20 (Table 2). Peptides that were not adjacent in the RBD sequence (e.g., peptides 18 and 20) could appear in close vicinity on the RBD surface.

Spike Protein-Specific Antibodies are Predominantly IgG and have Higher Titer in Patients Surviving Severe COVID-19

In a first set of experiments, IgG, IgG subclasses, IgM and IgA levels specific for folded S and RBD were measured in the complete population of COVID-19 convalescent patients (mild: n=139; severe; n=114) and in the 235 control subjects by ELISA. Severe COVID-19 patients had significantly higher IgG, IgM and IgA levels to S and RBD compared to mild COVID-19 patients. S- and RBD-specific IgG levels were higher than IgM levels, whereas few patients mounted low IgA responses. No significant correlations between S- and RBD-specific IgG, IgM and IgA responses were found.

In the control group 7.6% (n=18) had IgG to either S and/or RBD. Eleven subjects had COVID-19-like symptoms longer than 10 weeks before the visit, whereas in 7 subjects (i.e., 2.9%) no symptoms at all had been reported indicating a previous asymptomatic infection.

IgG subclass analysis revealed a predominant IgG₁ response to S and RBD with significantly higher IgG₁ levels in patients with severe COVID-19, compared to mild COVID-19 patients. In 23 COVID-19 patients a weak S-specific IgG₂ response was found but no S-specific IgG₃ or IgG₄ could be detected. S- and RBD-specific IgG levels were significantly correlated with IgG₁- but not IgG₂-levels.

### Twenty Percent of COVID-19 Patients Selectively Lack RBD-Specific IgG Responses

Out of the 253 COVID-19 patients 53 (i.e., 20.9%) lacked RBD-specific IgG antibodies. Among these RBD-non responders, there were more females (56.6% vs 43%) than among responders, their BMI was lower (24.7 vs 26.3) than in responders, and a significantly larger percentage of them had mild (75.5%) versus severe COVID-19 (24.5%). In contrast, the percentage of patients with mild and severe COVID-19 was identical in RBD-responders and their mean age (non-responders 51.1 vs responders 54.1 years) was comparable. Notably, the vast majority of the RBD non-responders (i.e., 83%) showed IgG reactivity to S and/or NP, 64.2% had IgG to S and NP and 18.9% only to NP. Only 17% of the non-responders lacked S- and NP-specific IgG.

Virus Neutralization in Patients is Associated with High Levels of IgG Against Conformational RBD Epitopes

For the assessment of antibody reactivity to a comprehensive panel of SARS-CoV-2 proteins and S-derived peptides a microarray technology was used (Ref. 19).

The IgG response in COVID-19 patients as assessed with microarrayed antigens was predominantly directed against folded S, RBD, S1 and S2. The highest antibody levels determined as ISAC standardized units (ISU) occurred towards folded proteins (folded S: 6.8 ISU-69.5 ISU, mean 34.4 ISU; folded RBD: 5.6 ISU-93.6 ISU; mean 72.5 ISU; folded S1: 0.4 ISU-31.4 ISU, mean 8.1 ISU; folded S2: 0.6 ISU-28.3 ISU, mean 8.5 ISU) whereas unfolded RBD, S1 and S2 showed negligible IgG reactivity (unfolded RBD: 0.2 ISU-3.4 ISU; mean 0.6 ISU; unfolded S1: 0.4 ISU-7.1 ISU, mean 1.3 ISU; unfolded S2: 0.3 ISU-5.4 ISU, mean 1.2 ISU. Only nucleocapsid protein (NP) showed IgG reactivity that was similar against folded and unfolded protein targets (NP folded mean: 34.4 ISU; NP unfolded mean: 44.6 ISU). IgG levels to most of the S-derived unfolded peptides including the RBD-derived peptides 13-20 were very low with mean IgG of much less than 10 ISU with the exception of four S2-derived peptides, peptide 25 (mean: 15.4 ISU), peptide 32 (mean: 11.1 ISU), peptide 33 (mean: 30.8 ISU), and peptide 46 (mean: 24.4 ISU). These peptides showed significantly higher IgG reactivity with sera from COVID-19 patients than with sera from historic controls. Two additional peptides (i.e., 7 and 21) stood out because they showed appreciable mean IgG levels (peptide 7: 2.0 ISU-66.8 ISU, mean: 8.8 ISU, peptide 21: 2.0 ISU-42.8 ISU, mean 6.8 ISU) and IgG levels from historic control sera were significantly higher than from COVID-19 patients.

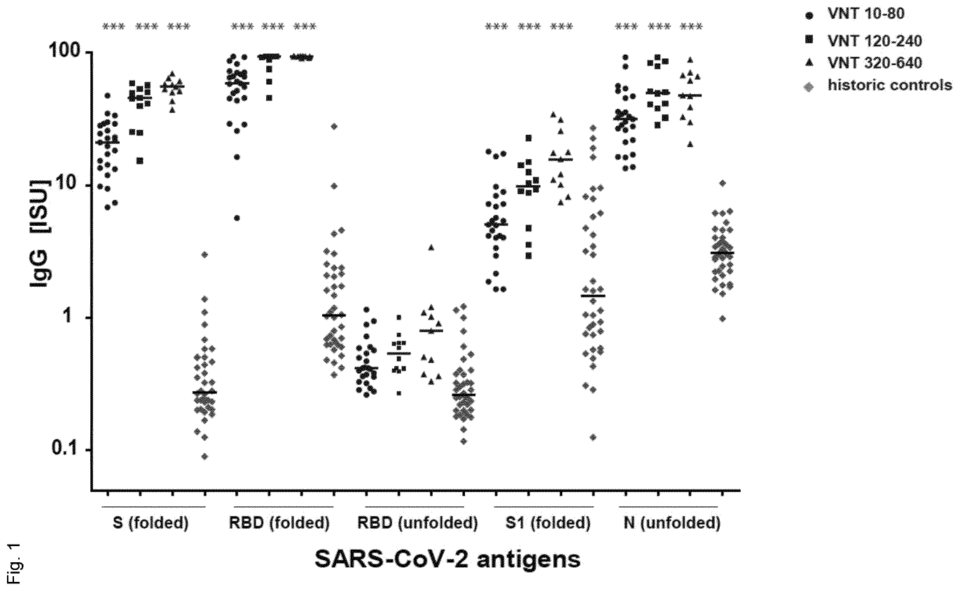

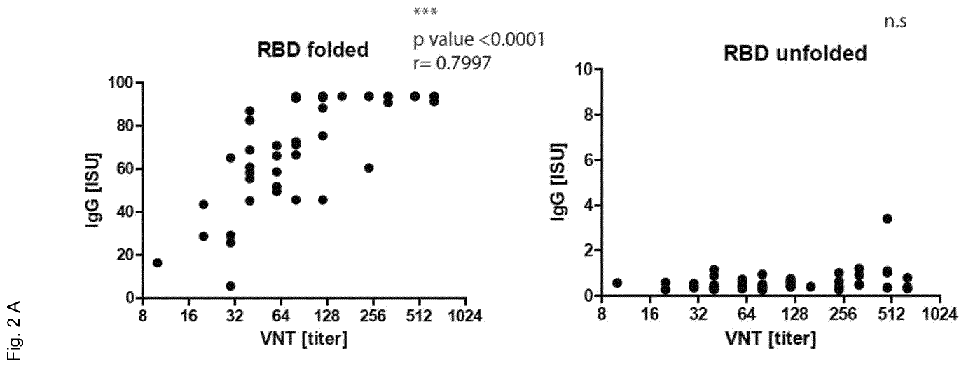

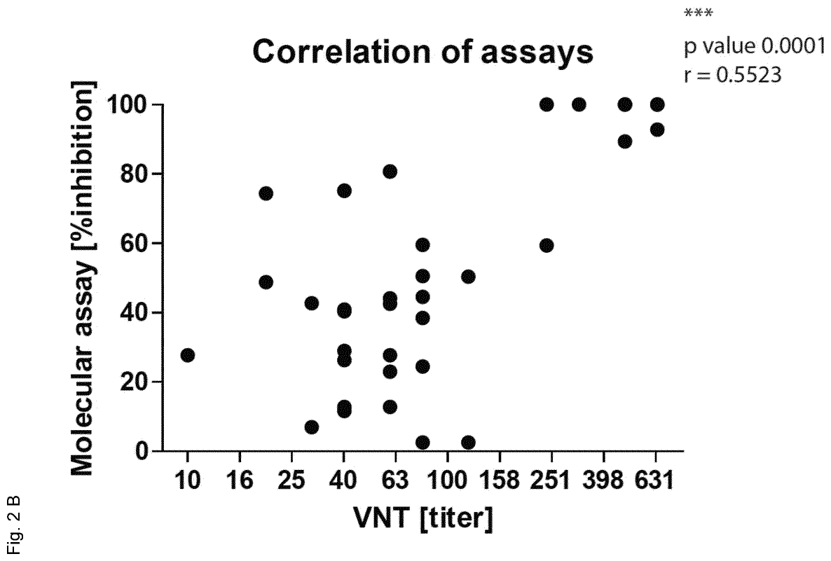

To correlate the virus neutralization titers of sera from COVID-19 patients with the response specificity, the patients were grouped according to their virus neutralization titres (VNTs) into three groups, VNT 10-80, VNT 120-240 and VNT 320-640, respectively. FIG. 1 shows that VNTs were correlated with IgG titers to folded S, S1 and in particular to folded RBD. IgG levels significantly increased with VNTs and were as follows: VNT 10-80: mean S-specific IgG: 21.1 ISU; mean S1-specific IgG: 3.7 ISU: mean RBD-specific IgG: 54.4 ISU; VNT 120-240: mean S-specific IgG: 42.1 ISU; mean S1-specific IgG: 10.1 ISU, mean RBD-specific IgG: 84.8 ISU; VNT 320-640: mean S-specific IgG: 54.4 ISU; mean S1-specific IgG: 15.4 ISU: mean RBD-specific IgG: 93.1 ISU (FIG. 1). High and significant correlations between VNTs and IgG levels to folded S, S1, S2 and RBD but not with IgG levels to unfolded S1, S2 or RBD were found (FIG. 2A). For RBD-derived peptides, no (peptides 13, 14, 15, 16, 18, 19, 21) or very low (peptides 17, 20) correlations were found between VNTs and specific IgG levels (FIG. 2A).

Specific IgG levels greater than 15 ISU and associations of specific IgG levels with VNTs were also found for NP which does not play a role in virus neutralization (FIG. 1) and for three S2-derived peptides (i.e., peptides 25, 33 and 46) which are outside RBD and hence are not directly involved in binding of RBD to ACE2.

Since VNTs were significantly correlated with levels of IgG antibodies to folded RBD in COVID-19 patients, it was analysed whether VNTs are associated with the ability of patients' sera to inhibit the binding of RBD to ACE2. FIG. 2B shows that there is indeed a highly significant correlation of VNTs with the inhibition of the binding of RBD to ACE2 in sera from COVID-19 patients.

An analysis of the ability of sera from 233 COVID-19 patients to block the binding of RBD to ACE2 was made. A median inhibition of RBD binding to ACE2 of 24% for this population was found. For 19.2% of patients a greater than 50% inhibition was found, in 38.4% of patients' inhibitions ranged from 20-50% and a less than 20% inhibition occurred in 42.4% of the patients.

Together these results demonstrate that neutralization of SARS-CoV-2 is associated with high levels of IgG antibodies against conformational epitopes of folded RBD and their ability to inhibit the binding of RBD to ACE2. However, the ability of patients' antibodies to inhibit RBD binding to ACE2 varied considerably.

### Only Folded RBD but not Sequential RBD Peptides Inhibit IgG Binding to Conformational RBD Epitopes

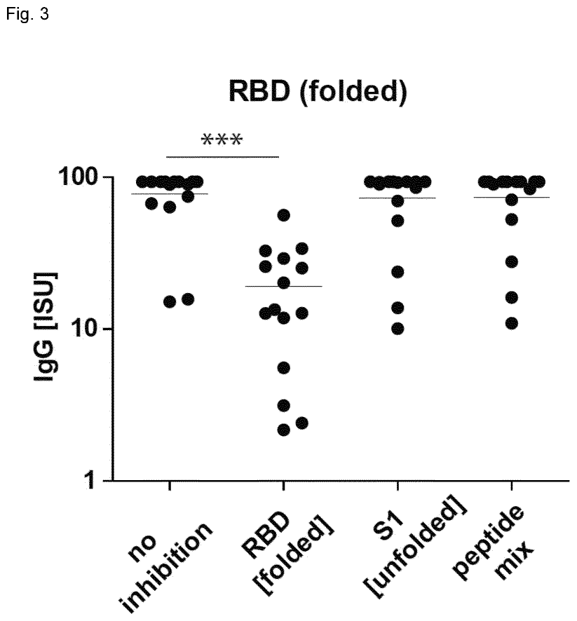

In order to further investigate the importance of conformational versus sequential RBD epitopes for the binding of patients' IgG to RBD, inhibition experiments were performed. Patients' sera were pre-incubated either with folded RBD containing conformational epitopes, with unfolded S1 or a mix of RBD-derived peptides containing sequential epitopes. For control purposes an unrelated protein (bovine serum albumin, BSA) was used. Then IgG binding of pre-adsorbed sera to folded RBD, folded S, unfolded S1, unfolded RBD and the RBD-derived peptides 13-21 was measured (FIG. 3). Only pre-incubation of sera with folded RBD, but not with unfolded S1 or the RBD-derived peptide-mix significantly inhibited IgG binding to conformational epitopes on RBD and reduced IgG binding to folded S (FIG. 3). Some non-significant reduction of the low IgG binding to unfolded RBD was observed after pre-incubation of sera with folded RBD, unfolded S1 and the RBD peptide mix (FIG. 3). Also, a non-significant reduction of the low IgG binding to unfolded S1 was observed by pre-adsorption with unfolded S1 and the RBD peptide mix (FIG. 3). Pre-incubation of sera with the RBD peptide mix reduced the low IgG binding to the individual RBD-derived peptides 13-21 with significant reductions observed for peptides 13, 17, 18, 20 and 21 (data not shown).

Immunization with Folded but not Unfolded RBD Induces Virus-Neutralizing Antibodies

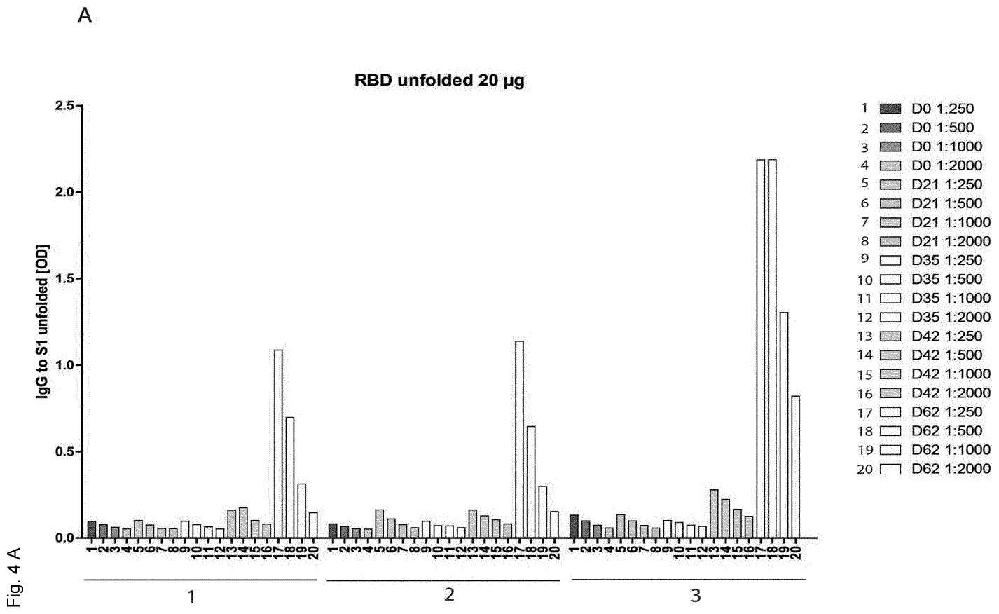

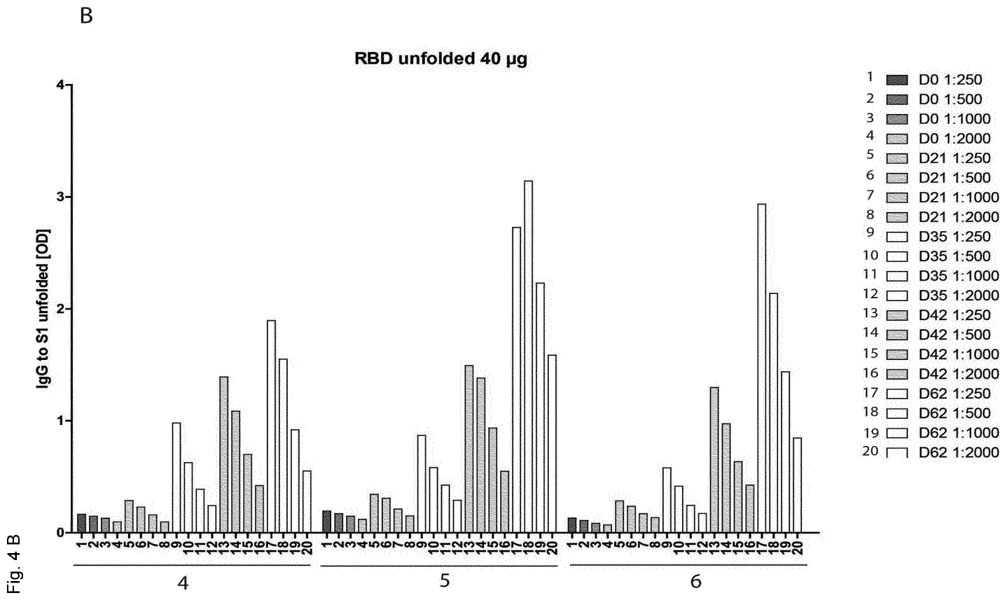

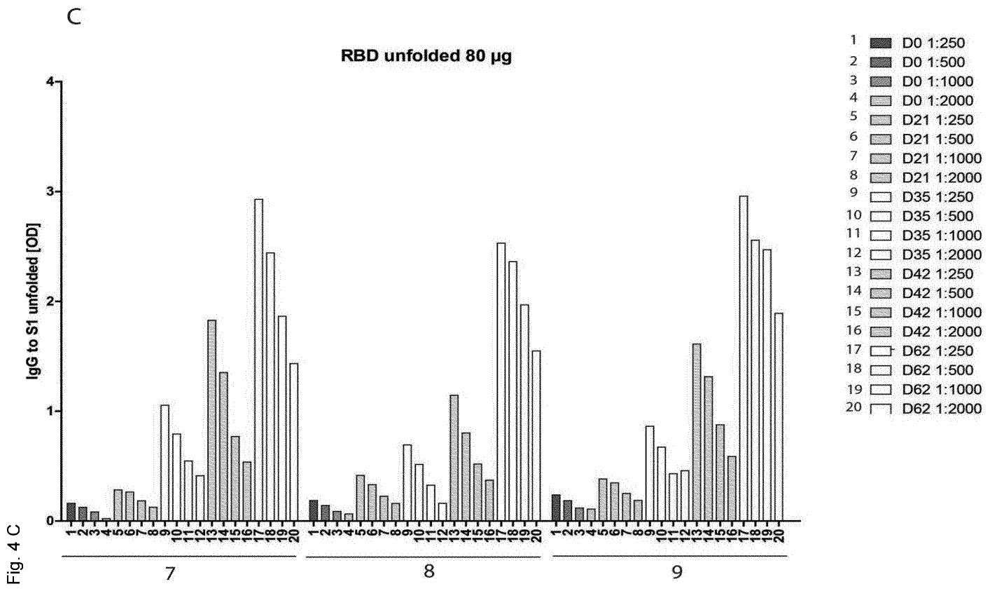

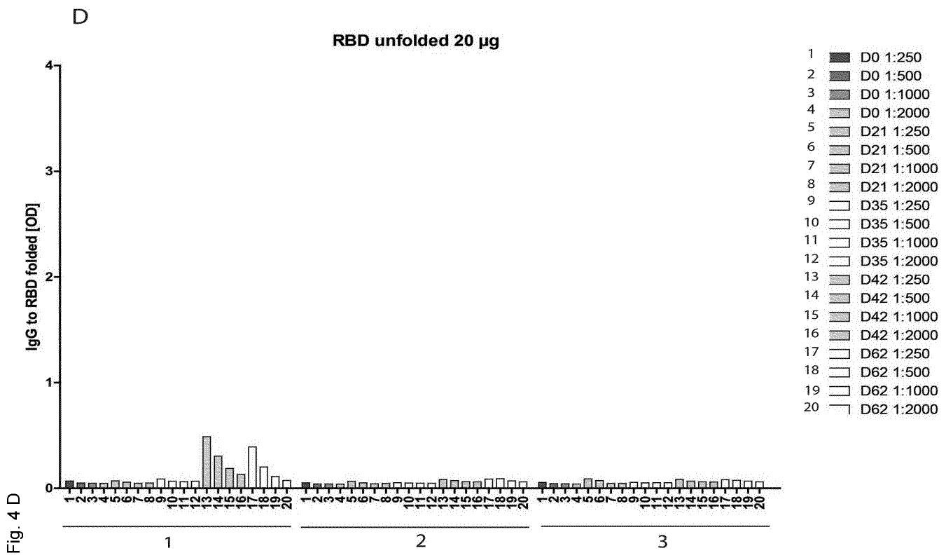

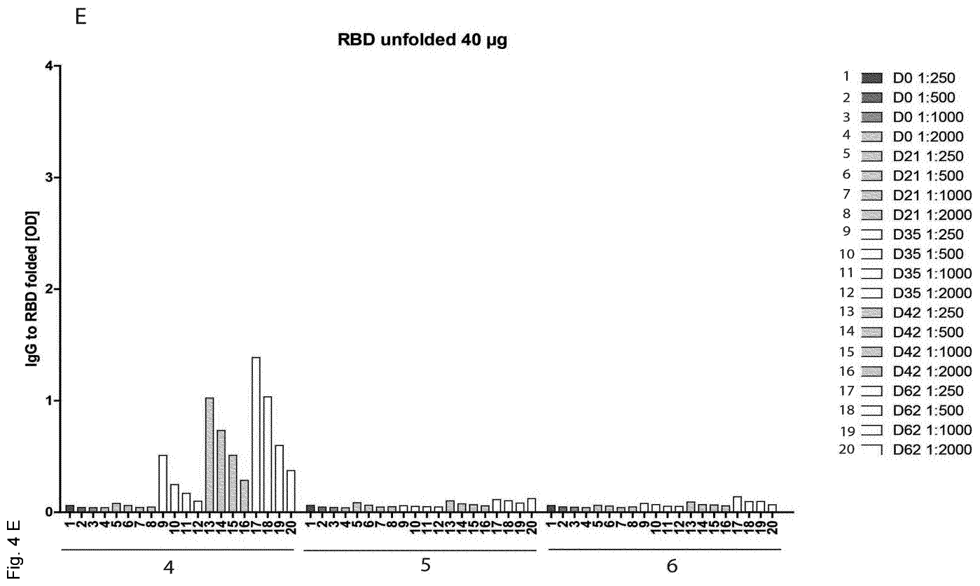

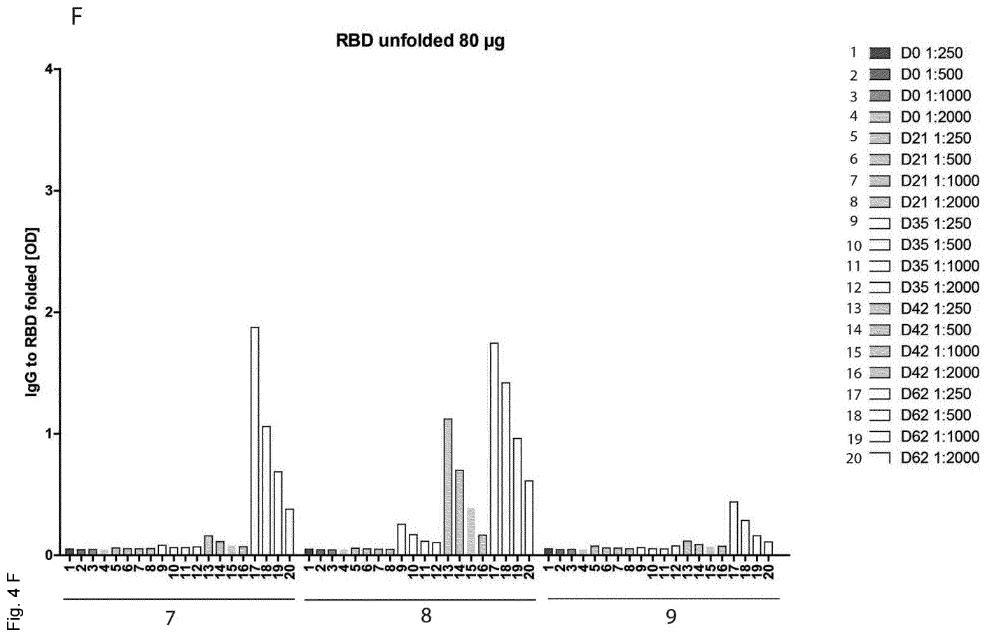

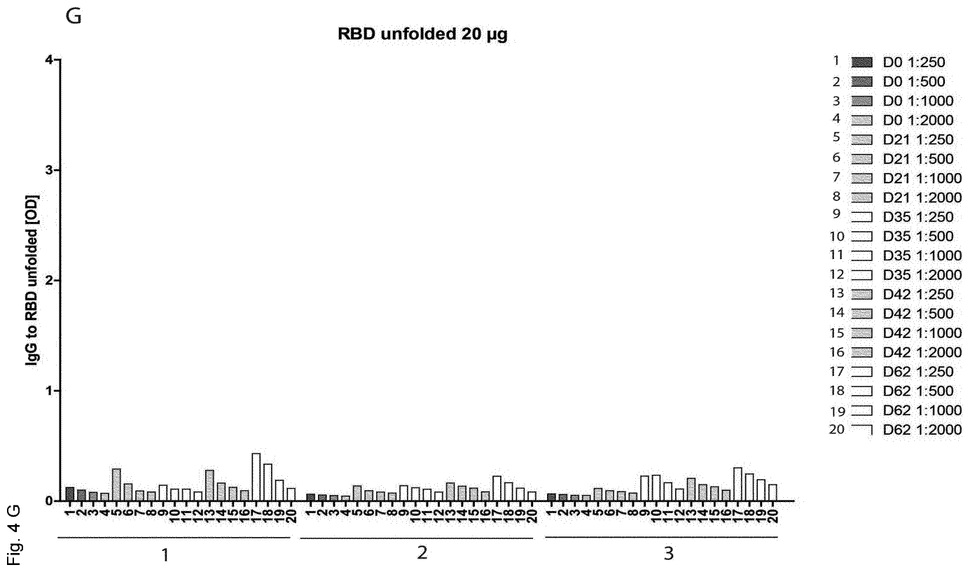

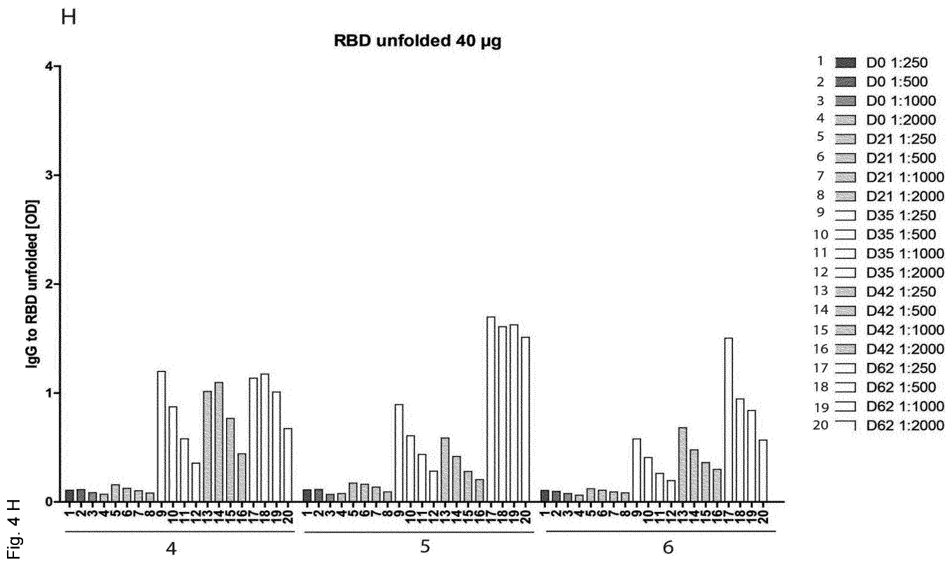

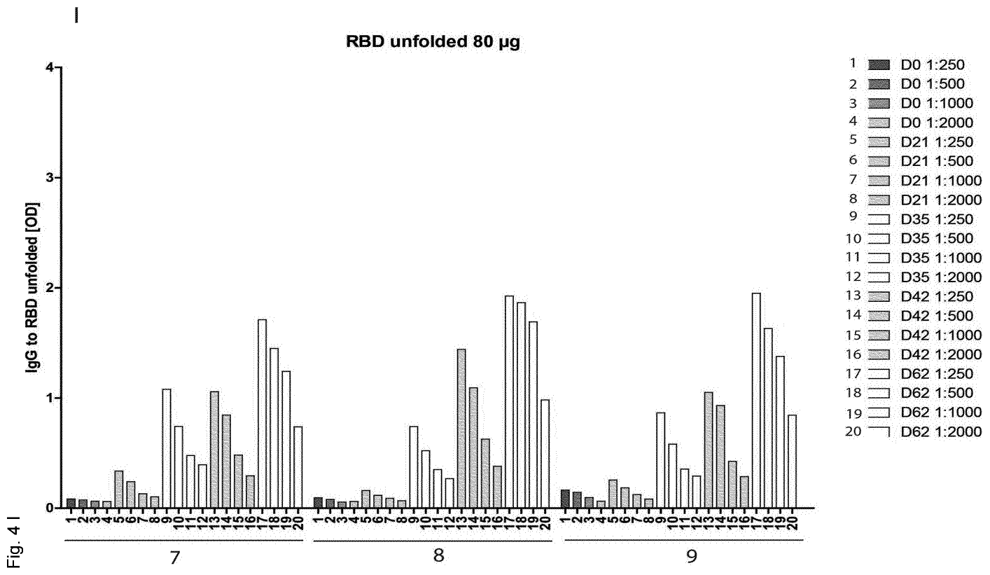

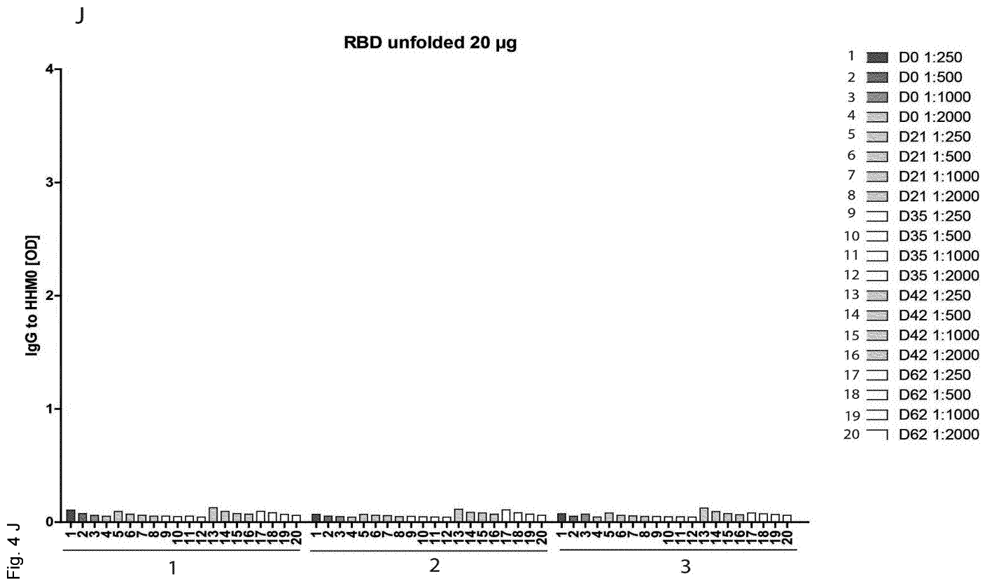

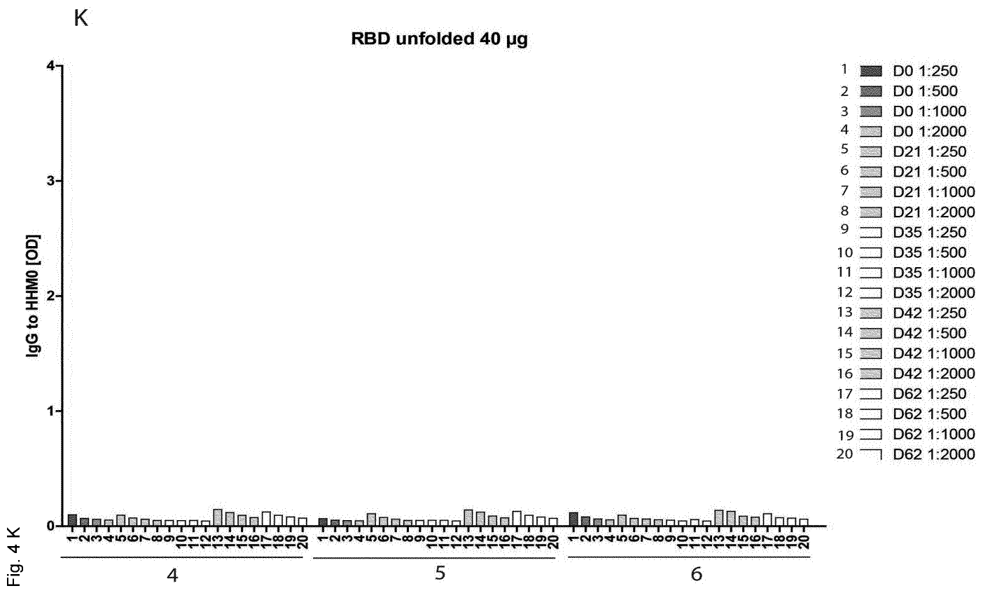

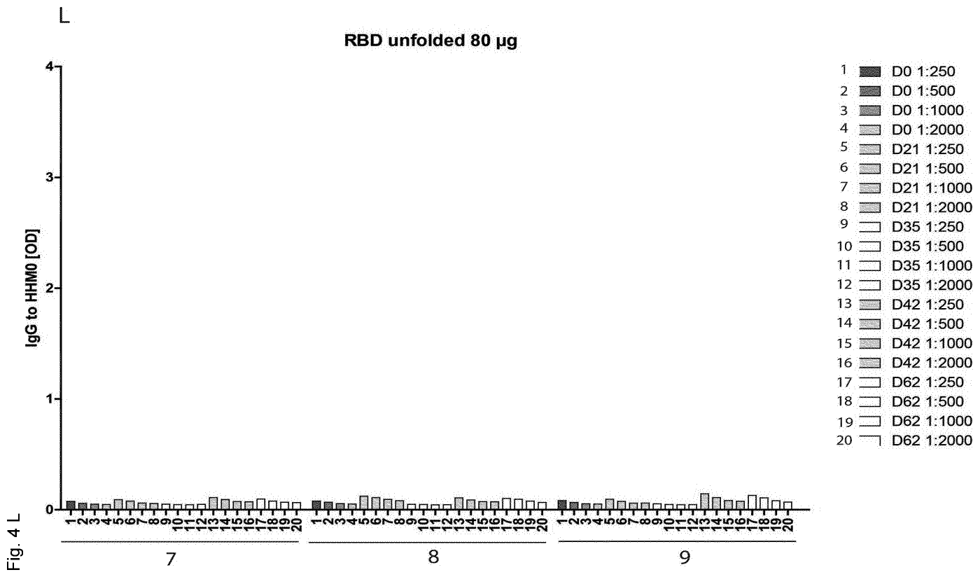

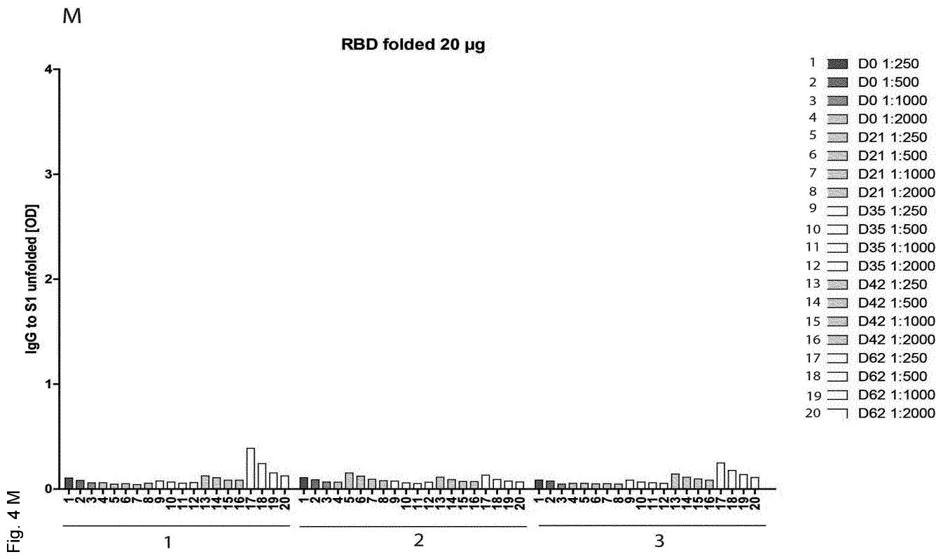

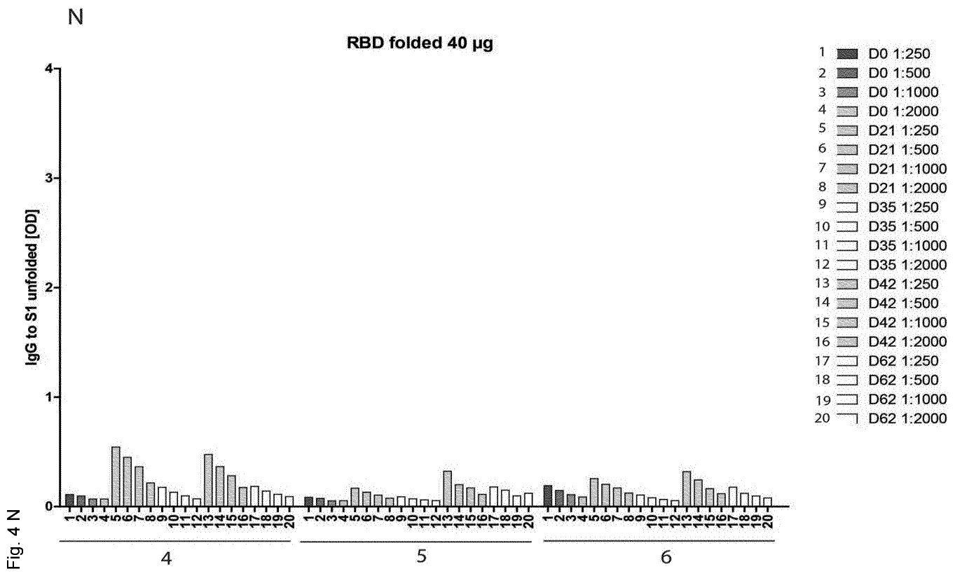

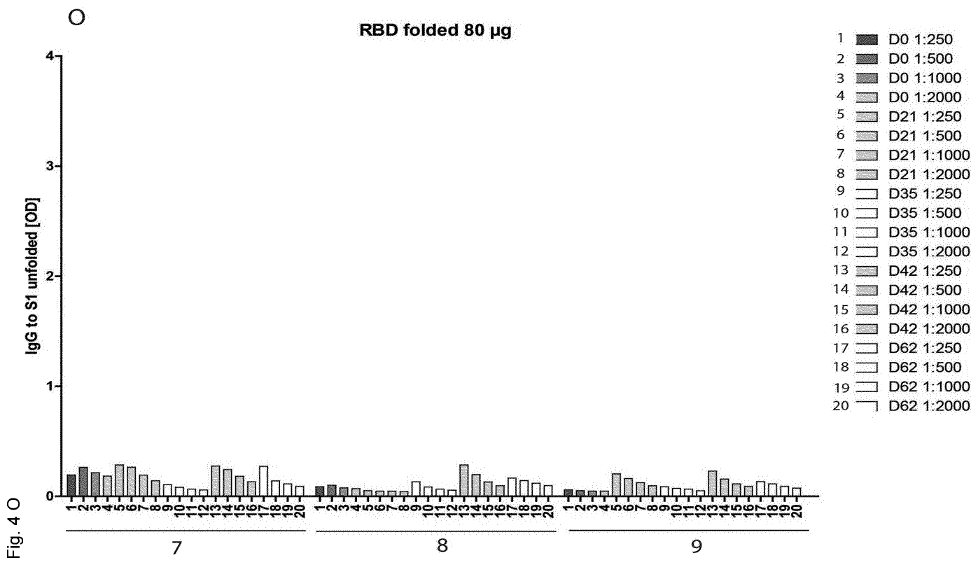

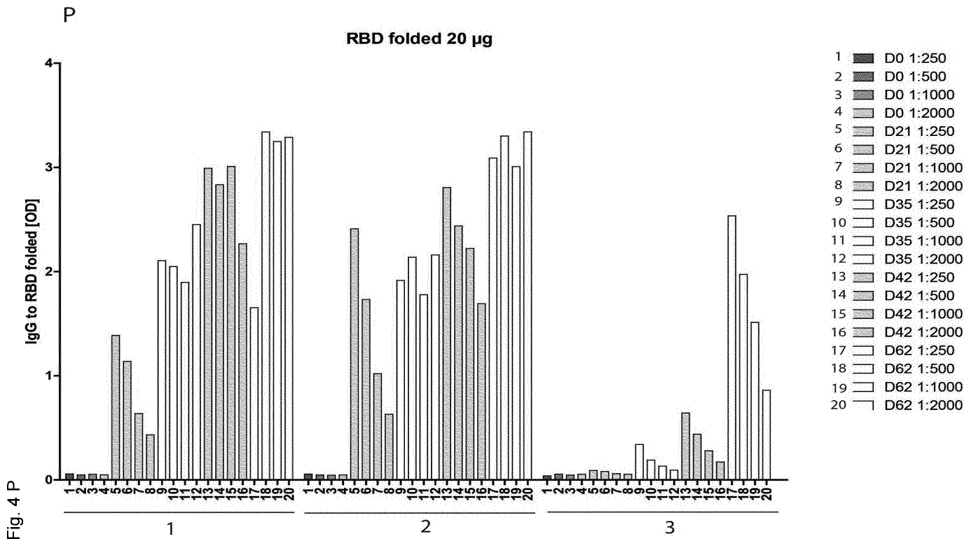

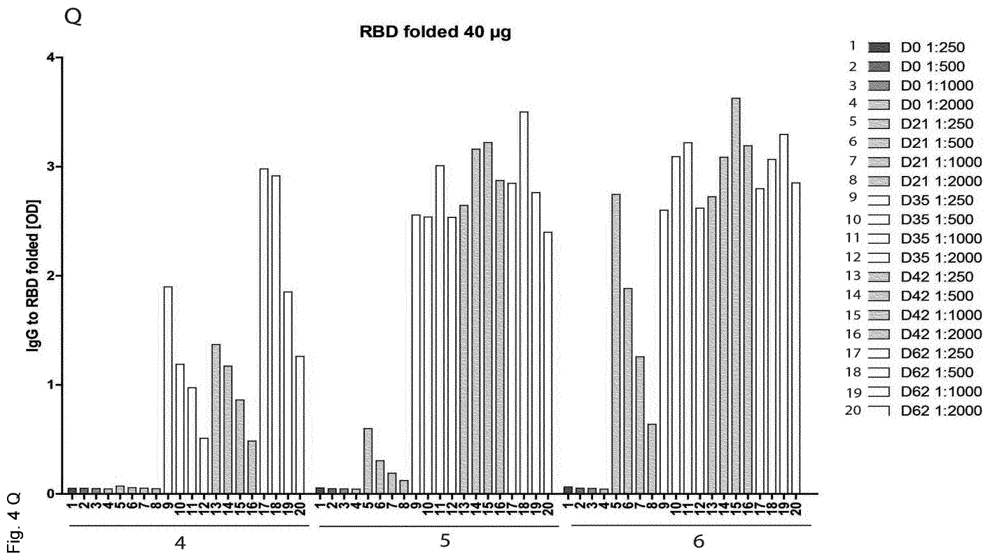

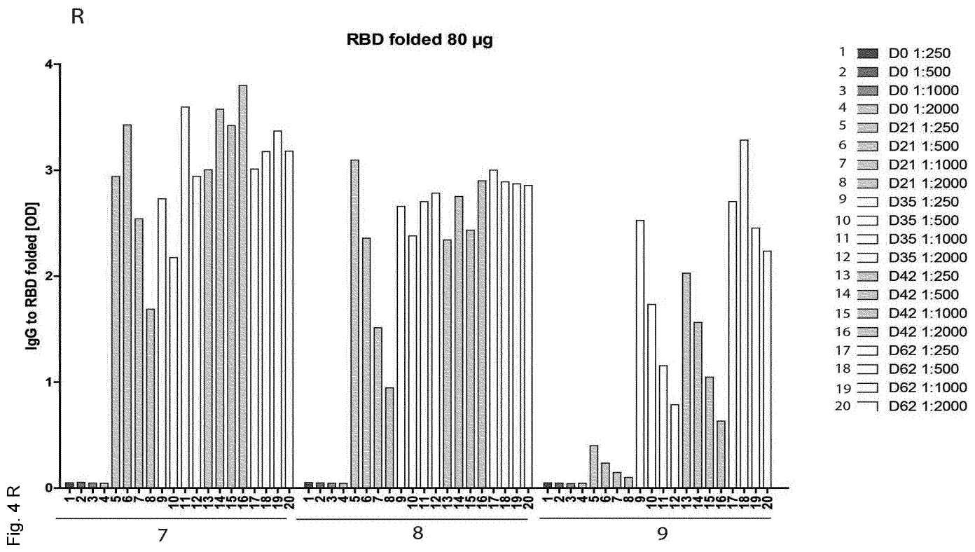

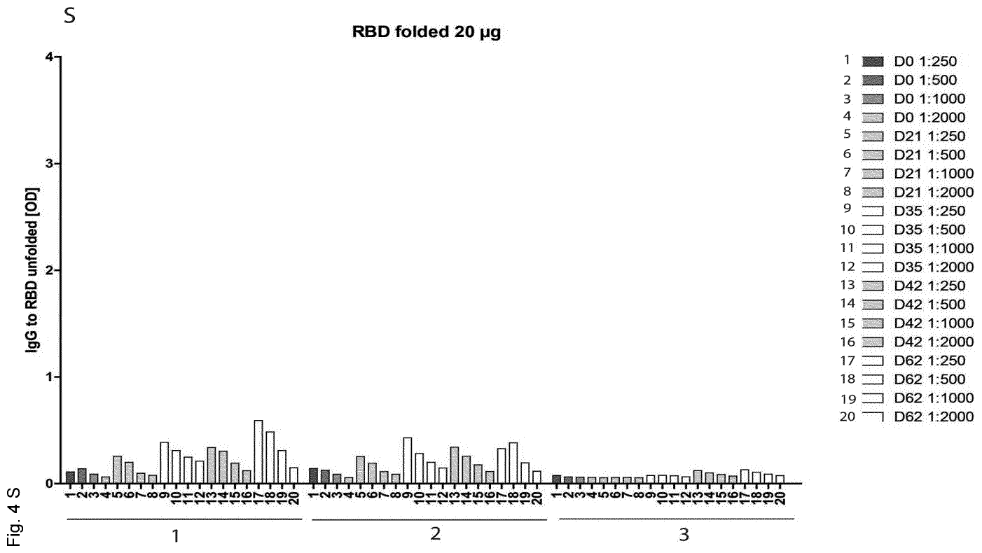

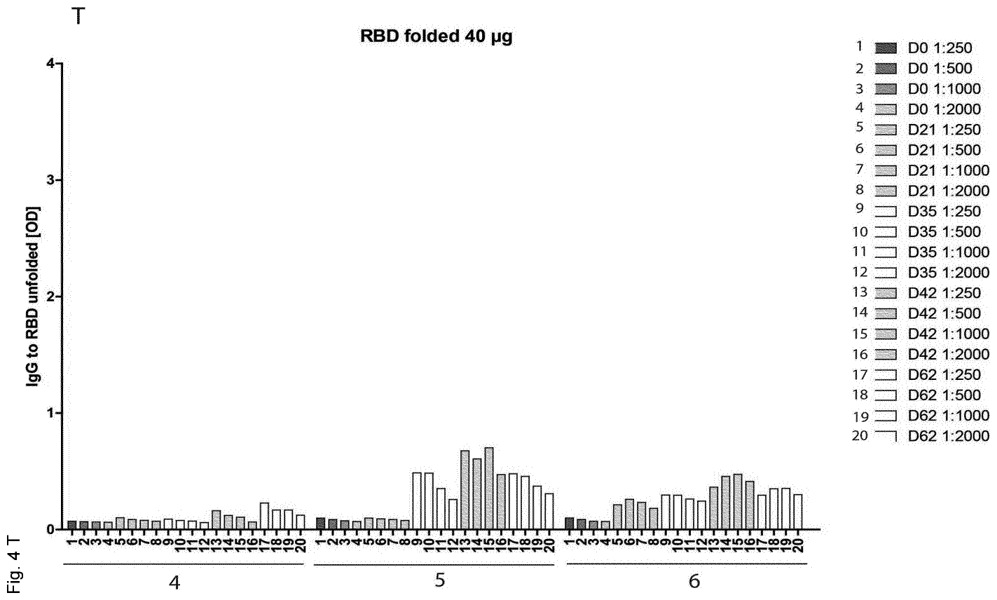

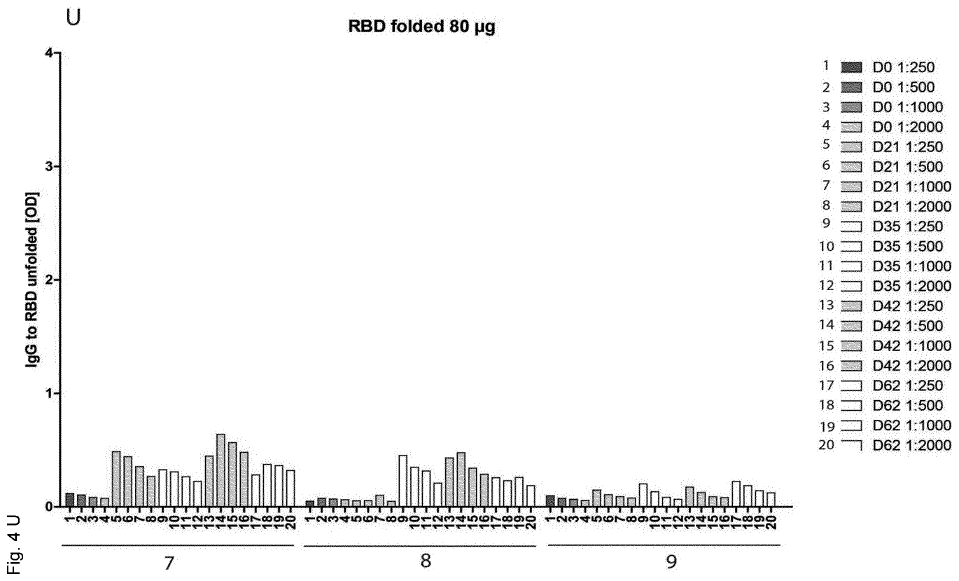

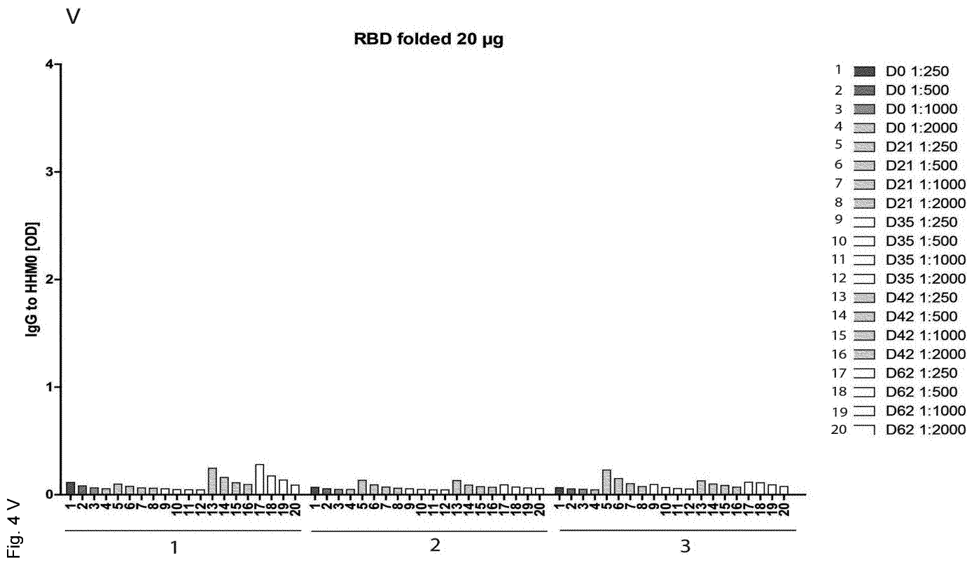

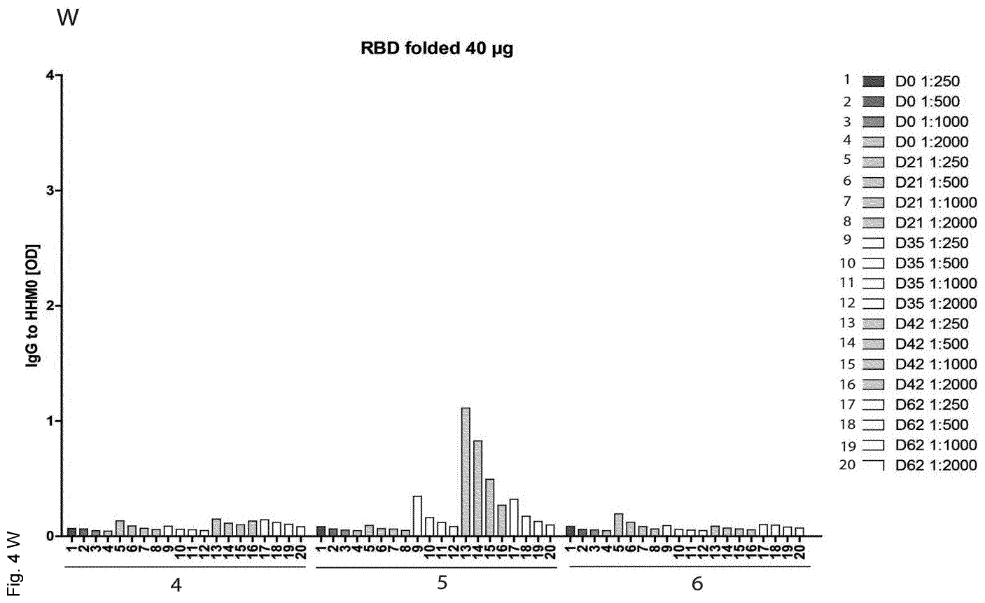

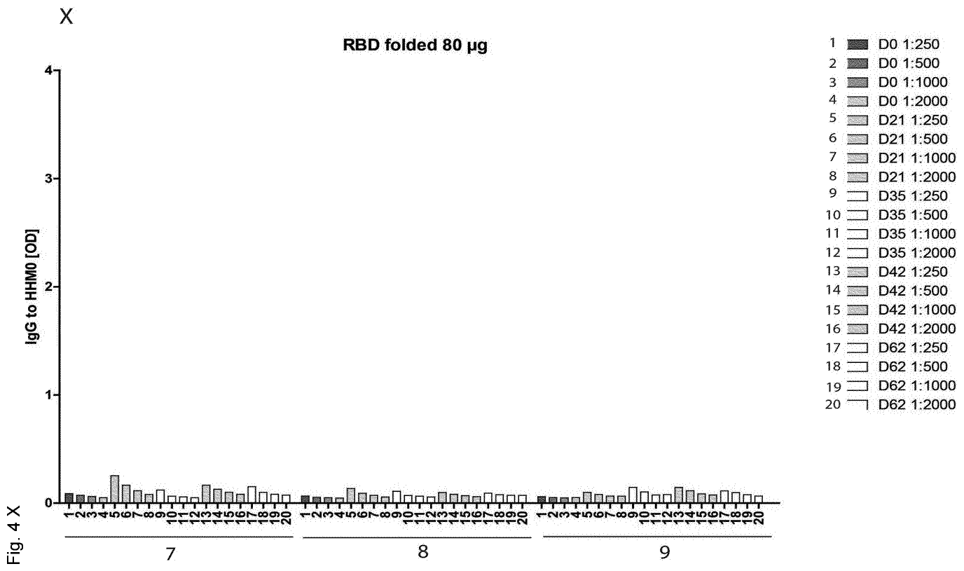

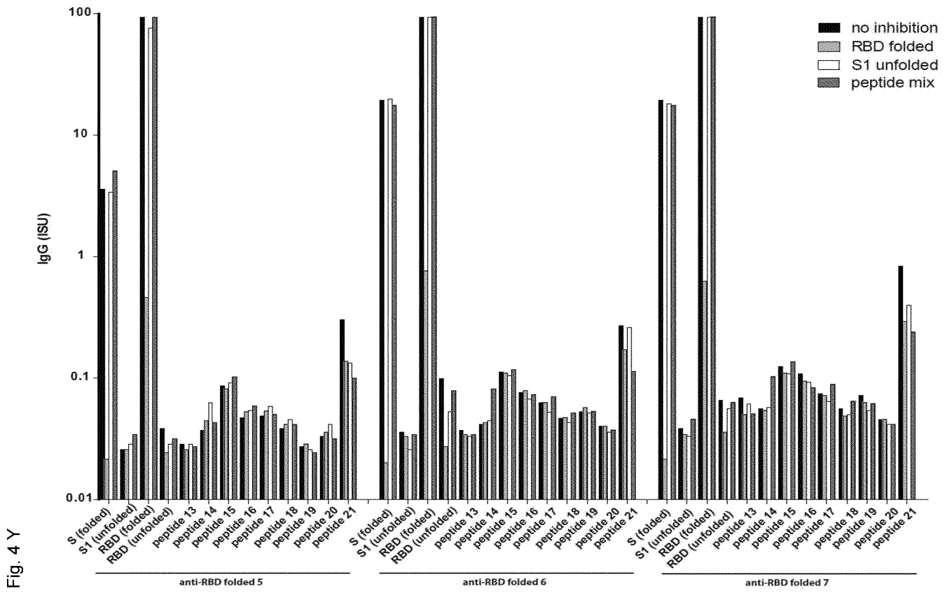

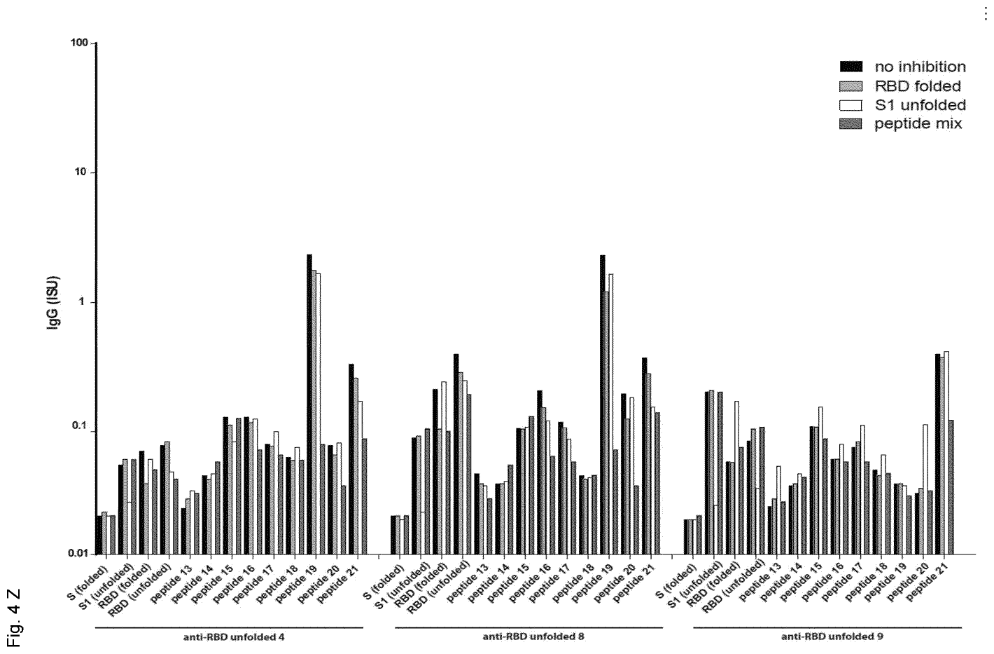

Immunization with denatured, synthetic or recombinant unfolded antigens can be used to induce antibodies recognizing the corresponding folded antigen to prevent and/or treat infectious diseases and allergy (20-23). Therefore, it was studied if immunization with unfolded RBD could induce IgG antibodies against folded RBD which exhibit high virus-neutralizing activity. Groups of rabbits were immunized with three doses (20, 40 or 80 microgram) of adjuvanted unfolded or folded RBD and, for control purposes with adjuvant alone. Immunization with unfolded RBD induced IgG reactivity to unfolded S1 and unfolded RBD but almost no IgG responses against folded RBD (FIG. 4), whereas immunization with folded RBD induces strong IgG production against folded RBD but almost no IgG antibodies against unfolded RBD and unfolded S1 (FIG. 4). No relevant IgG responses were observed in rabbits immunized with folded or unfolded RBD to an unrelated control antigen (HHM0) (FIG. 4) and no IgG response to any of the tested antigens was observed in rabbits immunized with adjuvant alone). The IgG reactivity of rabbits immunized with folded RBD to conformational epitopes on folded RBD and folded S was only inhibited by pre-adsorption with folded RBD but not with unfolded S1 or RBD-derived synthetic peptides containing only sequential epitopes (FIG. 4Y). The low IgG binding of rabbits immunized with unfolded RBD to unfolded proteins and RBD-derived peptides was only inhibited with unfolded S1 and/or RBD-derived peptides (FIG. 4Z).

Rabbit antisera obtained after the second and third immunization with folded or unfolded RBD for their VNTs were then tested (Table 4). With folded RBD (40 and 80 microgram) VNTs between 240->1280 were obtained after the third immunization whereas unfolded RBD failed to induce any VNT (Table 4). These results demonstrate that folded RBD containing conformational epitopes is required to induce high VNTs upon immunization.

### Discussion

The findings obtained in our population of COVID-19 patients are in agreement with another recent population study showing that the ACE2-binding site of SARS-CoV-2 RBD dominates the polyclonal neutralizing antibody response in COVID-19 patients (Ref. 9). However, our study provides important advances regarding the characteristics of a protective antibody response, and demonstrates how it can be induced by vaccination in experimental animals.

The analysis of sera from 253 COVID-19 convalescent patients shows that the antibody response against the spike protein and RBD is dominated by the IgG isotype, in particular by the IgG₁ subclass which is in agreement with an earlier report (Ref. 24). Using microarrayed folded S, folded and unfolded portions of the spike protein (S1, S2, RBD) as well as synthetic peptides spanning S, the present work shows that VNTs in patients' sera are highly correlated with the levels of IgG antibodies directed against conformational but not sequential RBD epitopes. In fact, the localization of the RBD-derived peptides in the three-dimensional structure of RBD shows that non-adjacent RBD-derived peptides appear in close vicinity on the RBD surface, which is required for the formation of conformational epitopes of the discontinuous type.

The finding, that the majority of the SARS-CoV-2 neutralizing activity of the polyclonal antibody response in COVID-19 convalescent patients can be attributed to IgG antibodies directed against conformational but not against sequential RBD epitopes is important because so far only 3 mutations have been observed in the RBD of currently reported SARS-CoV-2 variants of which only one (i.e., E484K) appears on the RBD surface but does not seem to be involved in the ACE2 interaction. It is thus likely that IgG antibodies from COVID-19 convalescent patients directed to the conformational RBD epitopes will cross-react with the currently emerging SARS-CoV-2 variants and confer cross-protection.

Another interesting result of our study is that 20% of patients lacked RBD-specific memory IgG responses although the majority of them elicited SARS-CoV-2-specific IgG antibodies directed against other epitopes on S and to NP. Possibilities for the selective lack of RBD-specific IgG memory responses include therefore genetic factors such as HLA restriction and/or insufficient T helper cell or B cell responses. Patients lacking RBD-specific memory IgG responses may be susceptible to repeated infections and propagate virus.

Lack of RBD-specific IgG in the non-responders did not seem to be a factor for severe disease because it was found that the majority of the RBD non-responders (i.e., 75.5%) had mild COVID-19. This may be due to low virus exposure of these subjects, sufficient early RBD-specific IgM responses, and/or a highly potent specific cellular immunity. In addition, a humoral immune response can be effective if it leads to complement fixation and lysis of the viral envelope or plasma membrane of infected cells, and hence a disruption of the interaction between virus and receptor is not a prerequisite for antiviral potency in general.

In fact, associations of VNTs with high specific antibody levels were noted also for NP and three S2-derived peptides. A role of NP-specific antibodies in virus neutralization is unlikely, whereas IgG against the S2-derived peptides may play a role in virus neutralization by inhibiting virus fusion. Analysis of the ability of patient sera to inhibit the binding of RBD to ACE2 in a molecular interaction assay revealed, that the ability of antibodies to inhibit the RBD binding to ACE2 was correlated with VNTs, confirming that antibodies against conformational RBD epitopes are predominantly responsible for the virus neutralization of the polyclonal antibody responses of COVID-19 patients, and not antibodies to S2-derived epitopes. The analysis of patients' sera regarding the presence of antibodies capable of inhibiting RBD binding to ACE in more than 230 patients is consistent with results obtained earlier in a smaller population showing that this blocking activity may vary considerably among patients (Ref. 25). Accordingly, antibodies against conformational RBD epitopes capable of inhibiting the RBD-ACE2 interaction seem to be an important parameter for the assessment of a protective SARS-CoV-2-specific immunity after disease or vaccination.

Other reports have shown that monoclonal antibodies or enriched antibody fractions specific for epitopes outside RBD or sequential epitopes may have SARS-CoV-2 neutralizing activity (Ref. 7,26). Although this information is valuable for the creation of therapeutic antagonists of the virus, it is of less certain relevance for the development of effective vaccine strategies, for which an understanding of the natural pattern of neutralizing responses and their therapeutic implications is important. In this context, it is worth mentioning that our analysis also confirmed the presence of low antibody responses against certain SARS-CoV-2 peptides in sera from historic controls obtained before the COVID-19 pandemic (Ref. 27).

Our result that the majority of virus-neutralizing activity in sera of SARS-CoV-2 patients can be attributed to antibodies against conformational RBD epitopes is supported by earlier studies performed for SARS-CoV which, similar to SARS-CoV-2, also binds with RBD to ACE2. Also for SARS-CoV it has been demonstrated that the spike protein contains conformational epitopes which induce highly potent neutralizing antibodies (Ref. 28). Furthermore, it has been shown that vaccines targeting the RBD of SARS-CoV-2 induce protective immunity (Ref. 29). However, for SARS-CoV it has been reported that potent neutralizing antibodies and protective immunity can be obtained by immunization with RBD expressed in a folded form in eukaryotic cells as well as with unfolded RBD, Escherichia coli-expressed RBD (Ref. 30). These results were consistent with data obtained for several vaccines for other infectious diseases and therapeutic vaccines for allergy demonstrating that one can induce protective antibody responses against the corresponding natural, folded antigen resembling conformational epitopes with the denatured antigens, the unfolded recombinant antigen or sequential peptides thereof (Ref. 15,16, 20-23). Conversely, it has been suggested for certain viral diseases that immunization with correctly folded antigens is required for obtaining protective antibody responses (Ref. 31,32).

In order to compare antibody responses obtained by immunization with folded versus unfolded RBD and their virus-neutralizing activity, rabbits were immunized with a folded and unfolded recombinant RBD protein. Only immunization with folded but not with unfolded RBD induced antibodies against conformational RBD epitopes and high VNTs.

Collectively our data demonstrate that the virus-neutralizing activity of antibodies in COVID-19 patients depends on the presence of antibodies directed to conformational epitopes of RBD, which do not seem to be altered in currently known mutated SARS-CoV-2 variants. However, not all COVID-19 patients develop these antibodies. Importantly, the induction of such antibodies by vaccination requires folded RBD. Thus, our results suggest that antibodies against conformational RBD epitopes are a surrogate marker for a SARS-CoV-2 neutralizing antibody response and are important for the development of SARS-CoV-2-specific vaccines capable of inducing sterilizing immunity.

### Tables

TABLE 1
SARS-COV-2 proteins spotted on the microarray

| | Calculated | Fold according to CD ¹ | | |
|---|---|---|---|---|
| | molecular | or manufacturers data | Expression | |
| Protein | weight [kDa] | sheet ² | system | Source |
| S (folded) | 135 | alpha helical/coiled coil ¹ | Insect cells | Genscript |
| S1 (folded) | 79 | alpha helical/coiled coil ¹ | HEK cells | Genscript |
| S1 (folded) | 79 | alpha helical/coiled coil ¹ | Insect cells | Genscript |
| S1 (unfolded) | 76 | unfolded ¹ | E.coli | in-house expression |
| S2 (folded) | 60 | folded ² | HEK cells | Native Antigen |
| | | | | Company |
| S2 (unfolded) | 56 | unfolded ¹ | E.coli | in-house expression |
| RBD (folded) | 30 | folded ¹ | HEK cells | Genscript |
| RBD (unfolded) | 62 | unfolded ¹ | E. coli | in-house expression |
| NP (folded) | 46 | folded ² | HEK cells | Biovendor |
| NP (unfolded) | 46 | unfolded ² | E.coli | Biovendor |

TABLE 2
SARS-COV-2 spike protein-derived peptides

| | | | | | Solvent |
|---|---|---|---|---|---|
| | | | | | accessible |
| | | | | Solvent | area in |
| | Amino acid sequence; | Molecular | | accessible | water/in |
| | Peptides 1-47 | weight | Calculated | area | protein |
| Peptide | (SEQ ID NO: 53-98) | [Da] | pI | [%] | [Å²] |
| 1 | PLVSSQCVNLTTRTQLPPAYTNSFTRGVYY | 3377.8 | 9.2 | n.d | n.d |
| 2 | RGVYYPDKVFRSSVLHSTQDLFLPFFSNVT | 3520.9 | 8.5 | n.d | n.d |
| 3 | FSNVTWFHAIHVSGTNGTKRFDNPVLPFND | 3418.7 | 6.9 | n.d | n.d |
| 4 | LPFNDGVYFASTEKSNIIRGWIFGTTLDS | 3249.6 | 4.5 | n.d | n.d |
| 5 | TLDSKTQSLLIVNNATNVVIKVCEFQFCND | 3357.8 | 4.5 | n.d | n.d |
| 6 | QFCNDPFLGVYYHKNNKSWMESEFRVYSSA | 3648.0 | 6.7 | n.d | n.d |
| 7 | VYSSANNCTFEYVSQPFLMDLEGKQGNFKN | 3431.8 | 4.6 | n.d | n.d |
| 8 | GNFKNLREFVFKNIDGYFKIYSKHTPINLV | 3603.1 | 9.7 | n.d | n.d |
| 9 | PINLVRDLPQGFSALEPLVDLPIGINITR | 3171.7 | 4.5 | n.d | n.d |
| 10 | NITRFQTLLALHRSYLTPGDSSSGWTAGAA | 3192.5 | 8.7 | n.d | n.d |
| 11 | TAGAAAYYVGYLQPRTFLLKYNENGTITDA | 3282.6 | 5.7 | n.d | n.d |
| 12 | TITDAVDCALDPLSETKCTLKSFTVEKGIY | 3262.7 | 4.4 | n.d | n.d |
| 13 | EKGIYQTSNFRVQPTESIVRFPNITNLC | 3255.7 | 8.2 | 41 | 3970/1614 |
| 14 | FNATRFASVYAWNRKRISNCVADYS | 2940.2 | 9.7 | 36 | 3426/1228 |
| 15 | VADYSVLYNSASFSTFKCYGVSPTK | 2735.0 | 8.1 | 36 | 2721/968 |
| 16 | VSPTKLNDLCFTNVYADSFVIRGDEVRQIA | 3371.8 | 4.6 | 16 | 3425/540 |
| 17 | VRQIAPGQTGKIADYNYKLPDDFTGCVIAW | 3340.8 | 6.0 | 20 | 2754/557 |
| 18 | CVIAWNSNNLDSKVGGNYNYLYRLFRKSNL | 3522.9 | 9.5 | 38 | 3774/1438 |
| 19 | KPFERDISTEIYQAGSTPCNGVEGF | 2746.0 | 4.4 | 53 | 2933/155 |
| 20 | GVEGFNCYFPLQSYGFQPTNGVGYQPYRVV | 3387.7 | 5.9 | 38 | 3586/1381 |
| 21 | PYRVVVLSFELLHAPATVCGPKKSTNLVKN | 3281.9 | 9.6 | 29 | 4174/1192 |
| 22 | NLVKNKCVNFNFNGLTGTGVLTESNKKFL | 3200.7 | 9.6 | n.d | n.d |
| 23 | PFQQFGRDIADTTDAVRDPQTLEILDIT | 3176.4 | 3.9 | n.d | n.d |
| 24 | ILDITPCSFGGVSVITPGTNTSNQVAVLY | 2967.3 | 3.8 | n.d | n.d |
| 25 | AVLYQDVNCTEVPVAIHADQLTPTWRVYST | 3390.8 | 4.5 | n.d | n.d |
| 26 | RVYSTGSNVFQTRAGCLIGAEHVNNSYECD | 3291.5 | 5.4 | n.d | n.d |
| 27 | SYECDIPIGAGICASYQTQTNSPRRARSVA | 3215.5 | 7.7 | n.d | n.d |
| 28 | TMSLGAENSVAYSNNSIANNSIAIPTNFTI | 3115.4 | 4.0 | n.d | n.d |
| 30 | TSVDCTMYICGDSTECSNLLLQYGSFCTQL | 3296.7 | 3.4 | n.d | n.d |
| 31 | FCTQLNRALTGIAVEQDKNTQEVFAQVKQI | 3393.8 | 6.1 | n.d | n.d |
| 32 | QVKQIYKTPPIKDFGGFNFSQILPDPSK | 3193.6 | 9.4 | n.d | n.d |
| 33 | PDPSKPSKRSFIEDLLFNKVTLADAGFIKQ | 3362.8 | 8.8 | n.d | n.d |
| 34 | GFIKQYGDCLGDIAARDLICAQKFNGLTVL | 3243.7 | 6.0 | n.d | n.d |
| 35 | TDEMIAQYTSALLAGTITSGW | 2229.4 | 3.6 | n.d | n.d |
| 36 | ITSGWTFGAGAALQIPFAMQMAYRFNGIGV | 3176.7 | 8.7 | n.d | n.d |
| 37 | NGIGVTQNVLYENQKLIANQFNSAIGKIQD | 3290.6 | 6.0 | n.d | n.d |
| 38 | GKIQDSLSSTASALGKLQDVVNQNAQALNT | 3072.3 | 5.9 | n.d | n.d |
| 39 | LNTLVKQLSSNFGAISSVLNDILSRLDK | 3046.5 | 8.5 | n.d | n.d |
| 40 | LDKVEAEVQIDRLITGRLQSLQTYVTQQ | 3245.6 | 4.7 | n.d | n.d |
| 41 | YVTQQLIRAAEIRASANLAATKMSECVL | 3051.5 | 8.2 | n.d | n.d |
| 42 | CVLGQSKRVDFCGKGYHLMSFPQSAPH | 2993.4 | 8.8 | n.d | n.d |
| 43 | PHGVVFLHVTYVPAQEKNFTTAPAICHDGK | 3277.7 | 7.3 | n.d | n.d |
| 44 | CHDGKAHFPREGVFVSNGTHWFVTQRNFYE | 3566.9 | 7.0 | n.d | n.d |
| 45 | RNFYEPQIITTDNTFVSGNC | 2319.5 | 4.3 | n.d | n.d |
| 46 | NNTVYDPLQPELDSFKEELDKYFKNHT | 3285.5 | 4.5 | n.d | n.d |
| 47 | FKNHTSPDVDLGDISGINASVVNIQKEI | 3011.3 | 4.6 | n.d | n.d |

TABLE 3
Control proteins used in the microarray

| | | Natural/ | Expression | |
|---|---|---|---|---|
| Protein ID | Protein | recombinant | system/origin | Source |
| Gal d 1 | Ovomucoid | natural | Chicken | Sigma |
| Gal d 2 | Ovalbumin | natural | Chicken | Sigma |
| Gal d 4 | Lysozyme | natural | Chicken | Sigma |
| Gal d 5 | Albumin | natural | Chicken | Sigma |
| Bos d LF | Lactoferrin | natural | Cow | Sigma |
| Bos d 8 | Casein | natural | Cow | Sigma |
| BSA | Albumin | natural | Cow | Sigma |
| HSA | Albumin | natural | Human | Sigma |
| ACE2 | Angiotensin | recombinant | HEK cells | Genscript |
| | converting | | | |
| | enzyme | | | |
| HRP | Horseradish | natural | Horseradish | Sigma |
| | peroxidase | | | |
| HHM2 | Glycosylation | recombinant | Insect cells | In-house |
| | marker | | | expression |
| HHM0 | Glycosylation | recombinant | Insect cells | In-house |
| | marker | | | expression |
| IgG (c3) | hIgG 500 μg/ml | natural | Human | Sigma |
| IgG (c4) | hIgG 250 μg/ml | natural | Human | Sigma |
| IgG (c5) | hIgG 125 μg/ml | natural | Human | Sigma |
| IgG (c6) | hIgG 75 μg/ml | natural | Human | Sigma |
| IgG (c7) | hIgG 37.5 μg/ml | natural | Human | Sigma |

TABLE 4
Virus neutralization titers of rabbits immunized with folded and unfolded RBD

| | Rabbit | Day after 1^{st} | | Day after 1^{st} | |
|---|---|---|---|---|---|
| Antigen-μg | no. | immunization | VNT-Titer | immunization | VNT-Titer |
| RBD unfolded-20 | 1 | 42 | <10 | 63 | <10 |
| RBD unfolded-20 | 2 | 42 | <10 | 63 | <10 |
| RBD unfolded-20 | 3 | 42 | <10 | 63 | <10 |
| RBD unfolded-40 | 4 | 42 | <10 | 63 | <10 |
| RBD unfolded-40 | 5 | 42 | <10 | 63 | <10 |
| RBD unfolded-40 | 6 | 42 | <10 | 63 | <10 |
| RBD unfolded-80 | 7 | 42 | <10 | 63 | <10 |
| RBD unfolded-80 | 8 | 42 | <10 | 63 | <10 |
| RBD unfolded-80 | 9 | 42 | <10 | 63 | <10 |
| RBD folded-20 | 1 | 42 | 160 | 63 | 240 |
| RBD folded-20 | 2 | 42 | 240 | 63 | 320 |
| RBD folded-20 | 3 | 42 | 10 | 63 | 40 |
| RBD folded-40 | 4 | 42 | <10 | 63 | 60 |
| RBD folded-40 | 5 | 42 | 320 | 63 | 240 |
| RBD folded-40 | 6 | 42 | 320 | 63 | 640 |
| RBD folded-80 | 7 | 42 | ≥1280 | 63 | ≥1280 |
| RBD folded-80 | 8 | 42 | 480 | 63 | 480 |
| RBD folded-80 | 9 | 42 | 20 | 63 | 240 |
| Alum + buffer | 1 | 42 | <10 | 63 | <10 |
| Alum + buffer | 2 | 42 | <10 | 63 | <10 |
| Alum + buffer | 3 | 42 | <10 | 63 | <10 |

### REFERENCES OF EXAMPLE 5

1. Tang, D., Comish, P., & Kang, R. The hallmarks of COVID-19 disease. PLoS pathogens. 16, https://doi.org/10.1371/journal.ppat.1008536 (2020).
2. Morens, D. M., et al. The Origin of COVID-19 and Why It Matters. Am J Trop Med Hyg. 103, 955-959. doi: 10.4269/ajtmh.20-0849 (2020).
3. Li, F., Li, W., Farzan, M., & Harrison, S. C. Structure of SARS coronavirus spike receptor-binding domain complexed with receptor. Science. 309, 1864-1868. doi:10.1 126/science.1 116480 (2005).
4. V'kovski, P., Kratzel, A., Steiner, S., Stalder, H., & Thiel, V. Coronavirus biology and replication: implications for SARS-CoV-2. Nat Rev Microbiol. doi:10.1038/s41579-020-00468-6 (2020).
5. Liu, L., et al. Potent neutralizing antibodies against multiple epitopes on SARS-CoV-2 spike. Nature. 584, 450-456. doi:10.1038/s41586-020-2571-7 (2020).
6. Liu, S. T. H, et al. Convalescent plasma treatment of severe COVID-19: a propensity score-matched control study. Nat Med. 26, 1708-1713. doi:10.1038/s41591-020-1088-9 (2020).
7. Baum, A., et al. REGN-COV2 antibodies prevent and treat SARS-CoV-2 infection in rhesus macaques and hamsters. Science. 370, 1110-1115. doi:10.1126/science.abe2402 (2020).
8. Wajnberg, A., et al. Robust neutralizing antibodies to SARS-CoV-2 infection persist for months. Science. 370, 1227-1230. doi:10.1126/science.abd7728 (2020).
9. Piccoli L., et al. Mapping neutralizing and immunodominant sites on the SARS-CoV-2 spike receptor-binding domain by structure-guided high-resolution serology. Cell. 183, 1024-1042.e21. doi: 10.1016/j.cell.2020.09.037. Epub 2020 Sep. 16 (2020).
10. Krammer F. SARS-CoV-2 vaccines in development. Nature. 586, 516-527. doi:10.1038/s41586-020-2798-3 (2020).
11. Pollard A. J., & Bijker E. M. A guide to vaccinology: from basic principles to new developments. Nat Rev Immunol. 21, 83-100. doi: 10.1038/s41577-020-00479-7. Epub 2020 (2021).
12. McLellan, J. S., et al. Structure-based design of a fusion glycoprotein vaccine for respiratory
   syncytial virus. Science. 342, 592-598. doi:10.1126/science.1243283 (2013).
13. Sesterhenn, F., et al. De novo protein design enables the precise induction of RSV-neutralizing antibodies. Science. doi:10.1126/science.aay5051 (2020).
14. Gerlich, W. H. Medical virology of hepatitis B: how it began and where we are now. Virol J. doi:10.1186/1743-422X-10-239 (2013).
15. Tulaeva, I., et al. Quantification, epitope mapping and genotype cross-reactivity of hepatitis B preS-specific antibodies in subjects vaccinated with different dosage regimens of BM32. EBioMedicine. doi:10.1016/j.ebiom.2020.102953 (2020).
16. Ni Y., et al. Hepatitis B and D viruses exploit sodium taurocholate co-transporting polypeptide for species-specific entry into hepatocytes. Gastroenterology. 146, 1070-83. doi: 10.1053/j.gastro.2013.12.024. Epub 2013 Dec. 19 (2014).
17. Huang. C., et al. Clinical features of patients infected with 2019 novel coronavirus in Wuhan, China. Lancet. 395, 497-506. doi:10.1016/S0140-6736(20)30183-5 (2020).
18. Long, Q. X., et al. Antibody responses to SARS-CoV-2 in patients with COVID-19. Nat Med. 26, 845-848. doi:10.1038/s41591-020-0897-1 (2020).
19. Niespodziana, K., et al. PreDicta chip-based high resolution diagnosis of rhinovirus-induced wheeze. Nat Commun. 9, 2382. doi:10.1038/s41467-018-04591-0. (2018).
20. Volkman, D. J., Allyn & S. P., Fauci, A. S. Antigen-induced in vitro antibody production in humans: tetanus toxoid-specific antibody synthesis. J Immunol. 129, 107-112. (1982).
21. Sela, M. & Arnon, R. Synthetic approaches to vaccines for infectious and autoimmune diseases. Vaccine. 10, 991-999. doi:10.1016/0264-410x(92)90107-u. (1992).
22. Marsh, D. G., Lichtenstein, L. M. & Campbell, D. H. Studies on “allergoids” prepared from naturally occurring allergens. I. Assay of allergenicity and antigenicity of formalinized rye group I component. Immunology. 18, 705-722. (1970).
23. Valenta, R. The future of antigen-specific immunotherapy of allergy. Nat Rev Immunol. 2, 446-453. doi:10.1038/nri824. (2002).
24. Chen, Y., et al. A comprehensive, longitudinal analysis of humoral responses specific to four recombinant antigens of SARS-CoV-2 in severe and non-severe COVID-19 patients. PLoS Pathog. doi:10.1371/journal.ppat.1008796 (2020).
25. Gattinger, P., et al. Antibodies in serum of convalescent patients following mild COVID-19 do not always prevent virus-receptor binding. Allergy. 2021; 76(3):878-883. doi:10.1111/all.14523.
26. Li, Y., et al. Linear epitopes of SARS-CoV-2 spike protein elicit neutralizing antibodies in COVID-19 patients. Cell Mol Immunol. 17, 1095-1097. doi:10.1038/s41423-020-00523-5. (2020).
27. Shrock, E., et al. Viral epitope profiling of COVID-19 patients reveals cross-reactivity and correlates of severity. Science. doi:10.1126/science.abd4250. (2020).
28. He, Y., Lu, H., Siddiqui, P., Zhou, Y., & Jiang, S. Receptor-binding domain of severe acute respiratory syndrome coronavirus spike protein contains multiple conformation-dependent epitopes that induce highly potent neutralizing antibodies. J Immunol. 174, 4908-4915. doi:10.4049/jimmunol.174.8.4908. (2005).
29. Yang, J., et al. A vaccine targeting the RBD of the S protein of SARS-CoV-2 induces protective immunity. Nature. 586, 572-577. doi:10.1038/s41586-020-2599-8. (2020).
30. Du, L., et al. Recombinant receptor-binding domain of SARS-CoV spike protein expressed in mammalian, insect and E. coli cells elicits potent neutralizing antibody and protective immunity. Virology. 393, 144-150. doi:10.1016/j.virol.2009.07.018. (2009).
31. McLellan, J. S., et al. Structure-based design of a fusion glycoprotein vaccine for respiratory syncytial virus. Science. 342, 592-598. doi:10.1126/science.1243283. (2013).
32. Sesterhenn, F., et al. De novo protein design enables the precise induction of RSV-neutralizing antibodies. Science. doi:10.1126/science.aay5051. (2020).
33. Kratzer, B., et al. Immunological imprint of COVID-19 on human peripheral blood leukocyte populations. Allergy. doi:10.1111/all.14647. (2020).
34. Sonnweber, T., et al. Cardiopulmonary recovery after COVID-19—an observational prospective multi-center trial. Eur Respir J. 2020; 2003481. doi:10.1183/13993003.03481-2020. (2020).
35. Niespodziana, K., et al. A hypoallergenic cat vaccine based on Fel d 1-derived peptides fused to hepatitis B PreS. J Allergy Clin Immunol. 127, 1562-70. doi:10.1016/j.jaci.2011.02.004. (2011).
36. Gattinger, P., et al. Recombinant glycoproteins resembling carbohydrate-specific IgE epitopes from plants, venoms and mites. EBioMedicine. 39, 33-43. doi:10.1016/j.ebiom.2018.12.002. (2019).
37. Gallerano, D., et al. HIV microarray for the mapping and characterization of HIV-specific antibody responses. Lab Chip. 15, 1574-1589. doi:10.1039/c4lc01510j. (2015).
38. Borochova. K., et al. Features of the human antibody response against the respiratory syncytial virus surface glycoprotein G. Vaccines (Basel). doi:10.3390/vaccines8020337. (2020).
39. Koblischke, M., et al. Dynamics of CD4 T cell and antibody responses in COVID-19 patients with different disease severity. Front Med (Lausanne). doi:10.3389/fmed.2020.592629. (2020).

### Example 6: SARS-CoV-2 RBD Construct Comprising a VLP

### Methods

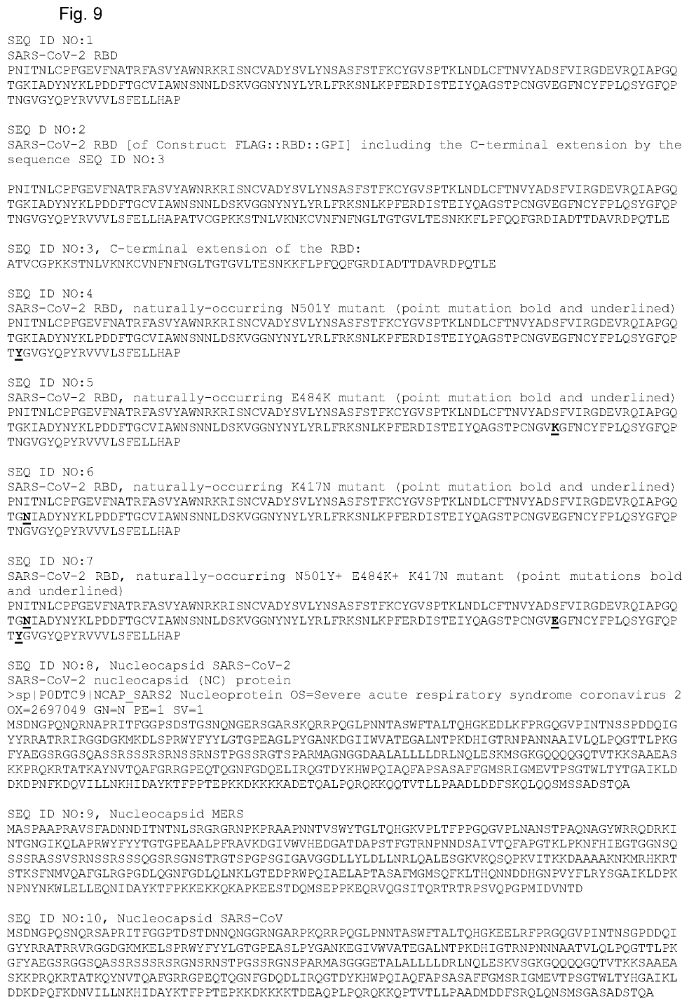

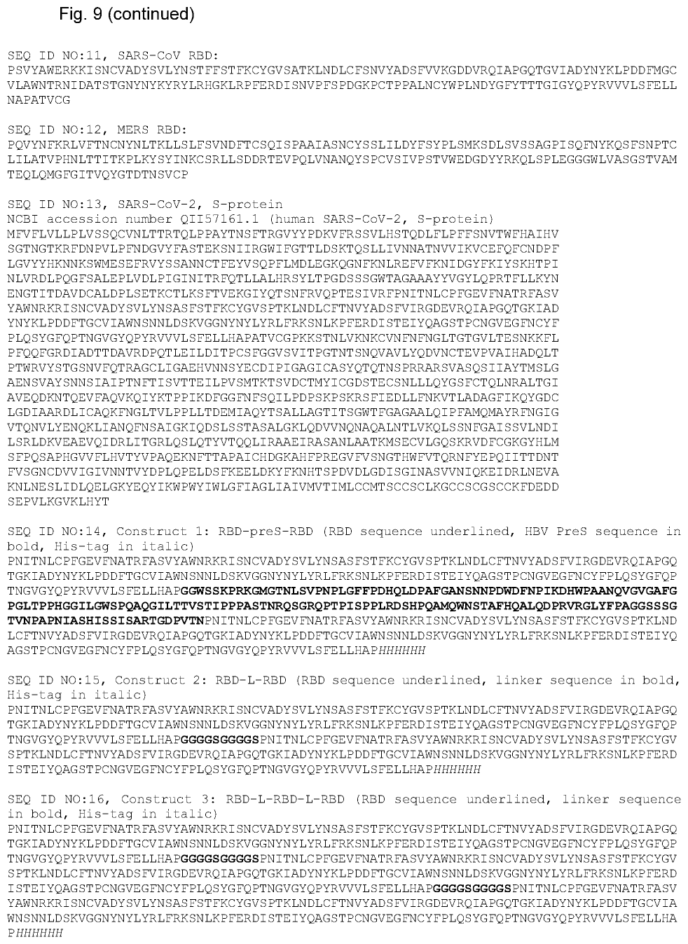

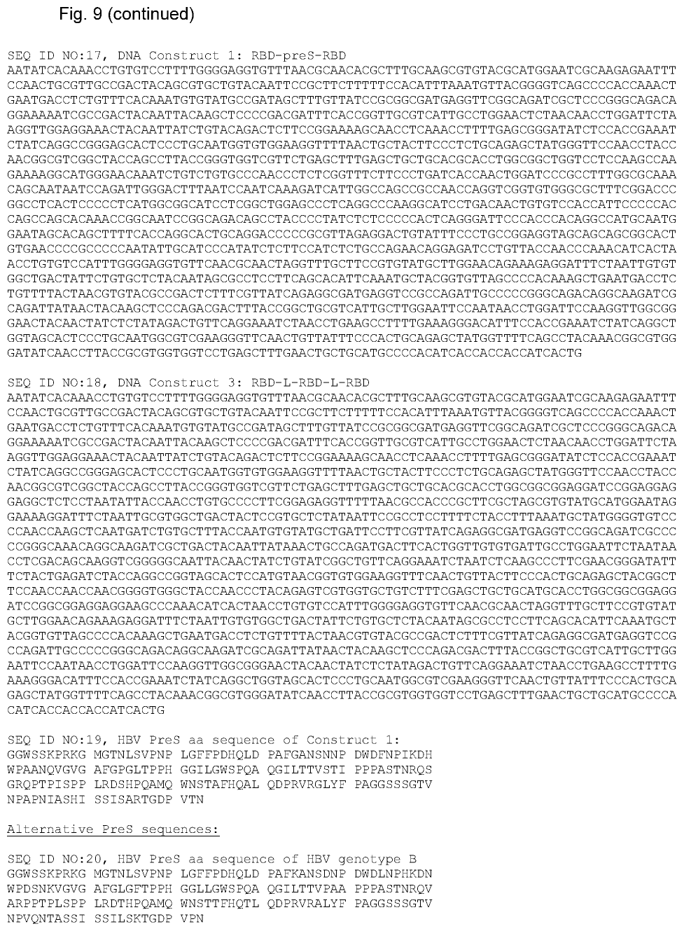

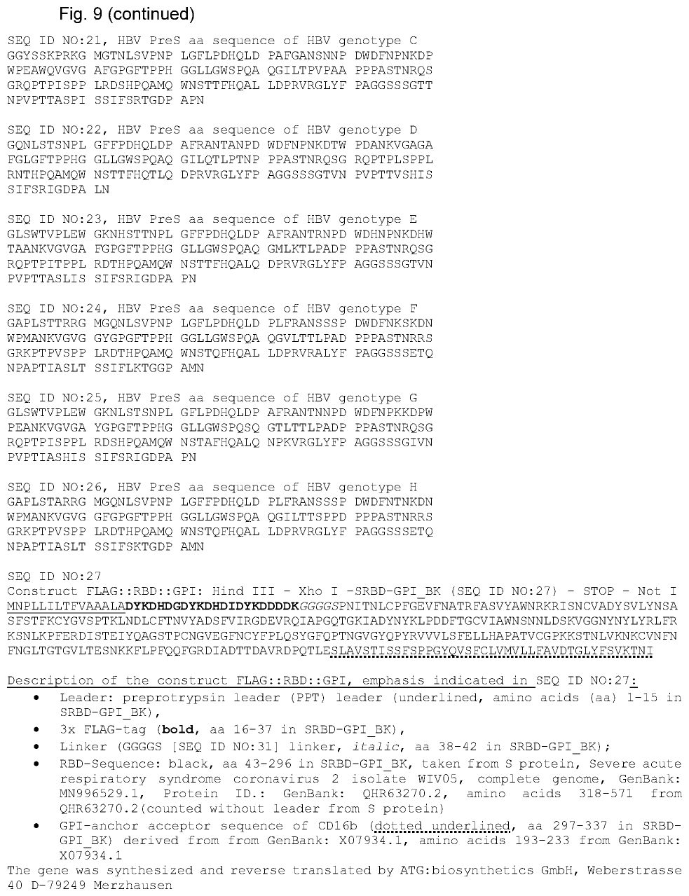

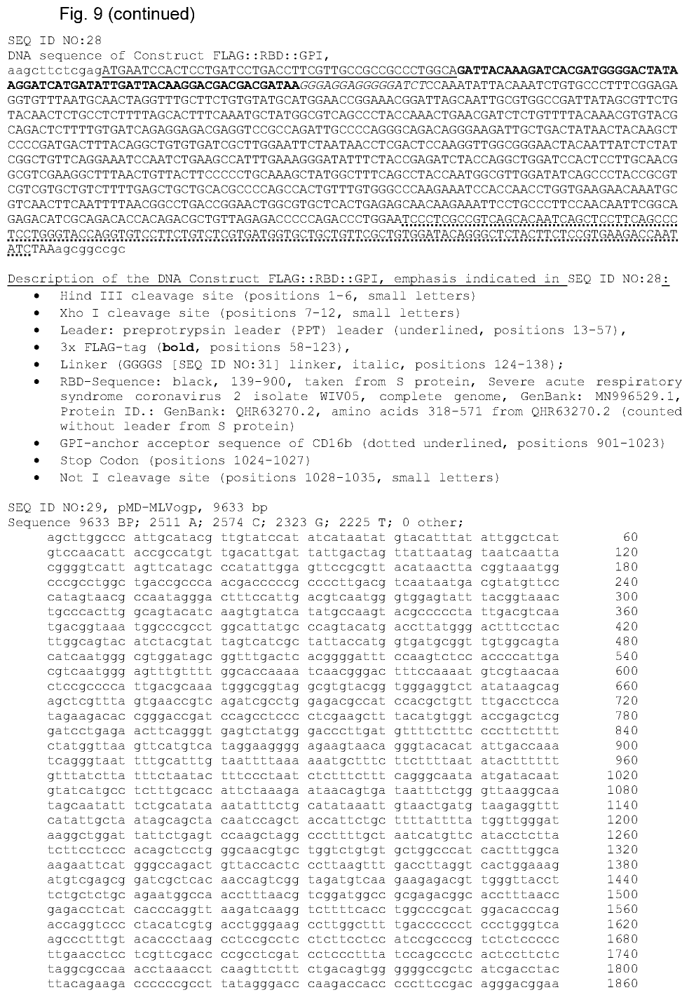

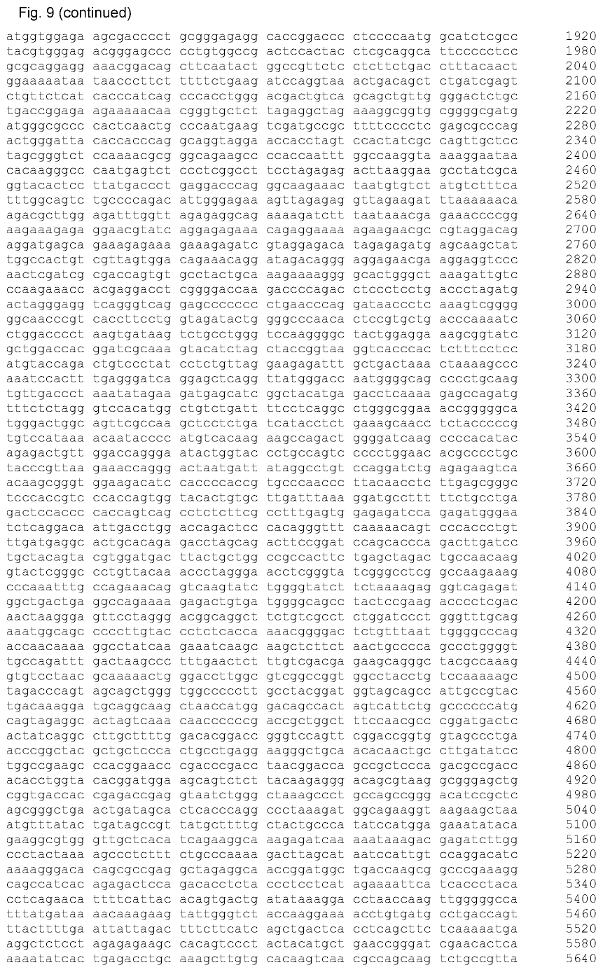

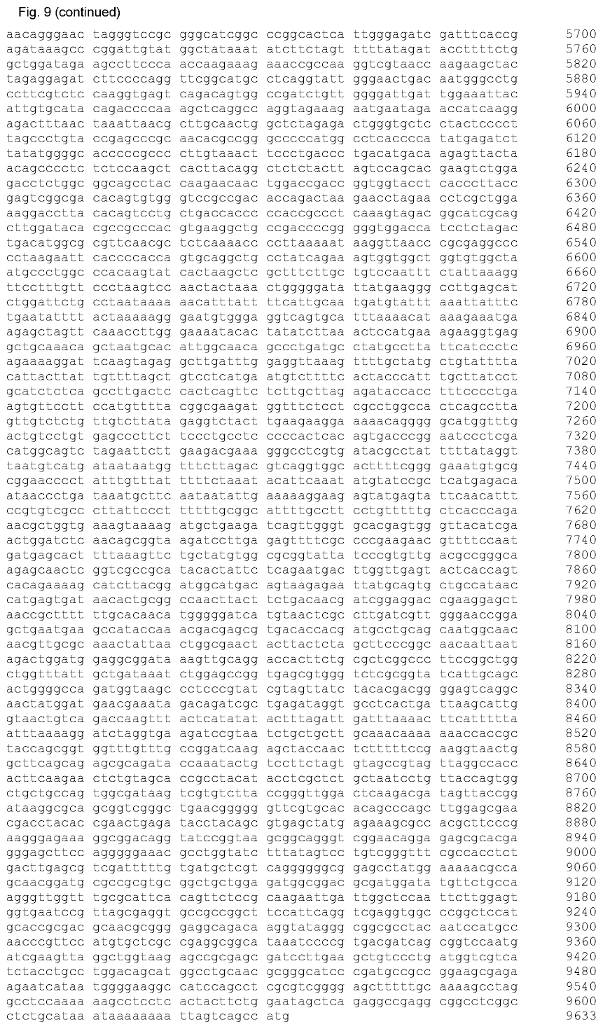

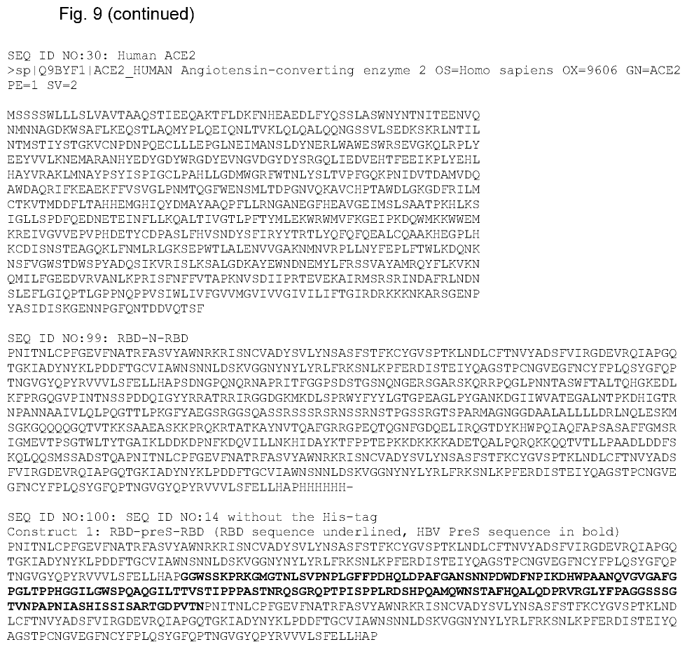

For the construct FLAG::RBD::GPI (amino acid sequence and DNA sequence, see FIG. 9), the preprotrypsin leader followed by a 3×FLAG tag (Ref. 40) and an GGGGS (SEQ ID NO:31) linker were fused to the RBD sequence from the S glycoprotein (taken from S protein, Severe acute respiratory syndrome coronavirus 2 isolate WIV05, complete genome, GenBank: MN996529.1, Protein ID.: GenBank: QHR63270.2, amino acids 318-571 from QHR63270.2, counted without leader from S protein) and fused on the C terminus to the minimal CD16b GPI anchor acceptor sequence, taken from GenBank: X07934.1, amino acids 193-233 from GenBank: X07934.1 (Ref. 41;42).

Expression was confirmed by transiently transfecting HEK293T cells with the calcium phosphate precipitation method. Briefly, 1×10⁶ HEK293T cells were seeded 24 hours before transfection in a petri dish (10 cm diameter, Sarstedt) in 10 ml of IMDM+10% FBS plus Gentamycin (15 mg/L). Two hours before the transfection, medium was exchanged with 8 ml of fresh IMDM+10% FBS plus Gentamycin (15 mg/L). For the transfection, 30 μg of pEAK12::FLAG::RBD::GPI construct was diluted in 900 μl of ddH₂O and mixed with 100 μl 2.5M CaCl₂) solution. Afterwards, 1 ml of 2×HBS (HEPES buffered saline, 140 mM NaCl, 1.5 mM Na₂HPO₄, 50 mM HEPES) buffer pH 7.0 was added dropwise to the DNA solution, incubated for one minute and then added dropwise to the cells. Thus, a total of 2 ml transfection mix was added per petri dish. Eighteen hours after transfection, the medium was replaced with 10 ml of fresh IMDM+10% FBS plus gentamycin and the cells were incubated for an additional 24 hours. In total, 48 hours after transfection, cells were harvested for flow cytometric analyses. For that purpose, cells were flushed off the plates with PBS (without Ca²⁺ and Mg²⁺), and washed twice with PBS. 5×10⁵ cells per staining were pipetted into a 4.5 ml polystyrene FACS tubes (BD) and first incubated for 10 minutes at room temperature with 0.1 μl Aqua Zombie (Biolegend). Afterwards, cells were washed with 4.5 ml of FACS-buffer (PBS plus 0.5% BSA and 0.05% NaN₃), centrifuged for 5 minutes with 500 g at 4° C. and the supernatant was discarded. Twenty microliters of 1:100 diluted sera of either a COVID-19 convalescent or a healthy control individual in FACS buffer were added to each staining, incubated for 30 minutes on ice and again washed with 4.5 ml of FACS buffer as described above. As a secondary antibody, 20 μl of 1:100 diluted goat-anti human IgG (gamma chain specific)-APC conjugated Fab's (Jackson Immunoresearch Laboratories, West Grove, PA, USA) was incubated on ice for 30 minutes and cells were washed again afterwards. Subsequently, at least 1×10⁴ live cells (Aqua zombie negative singlets) were acquired on a FACS Fortessa flow cytometer (BD) equipped with the DIVA software package (BD) and analyzed with the FlowJo software.

The generation of the construct pMD.OGP was described by Ory et al. previously (Ref. 46). Briefly, for pMD.gagpol, PCR was performed with pCRIPenv- (Ref. 47) using the following pairs of primers: 5′-CGGAATTCATGGGCCAGACTGTTACC-3′ (SEQ ID NO:49) and 5′-AGCAACTGGCGATAGTGG-3′ (SEQ ID NO:50), 5′-CGGAATTCTTAGGGGGCCTCGCGG-3′ (SEQ ID NO:51) and 5′-ACTACATGCTGAACCGGG-3′ (SEQ ID NO:52). The PCR products were digested with EcoRI and XhoI and with EcoRI and HindIII, respectively, to generate 0.94-kb Eco RI-Xho I and 0.94-kb HindIII-EcoRI fragments. These fragments were ligated with the 3.3-kb Xho I-Hind III fragment from pCRIPenv- and with pUC19, which had been linearized with Eco RI and calf intestinal phosphatase treated, to produce pUC19.gagpol. The 5.2-kb Eco RI fragment from pUC19.gagpol was cloned into the Eco RI cloning site in pMD to yield pMD.gagpol. pMD was constructed with the 3.1-kb Eco RI-Bam HI fragment from pBC12/CMV/interleukin 2 that includes the pXF3 backbone and HCMV enhancer promoter region and the previously described 1.34-kb Bam HI Xba I fragment derived from pUCMd,Bs(R)S. The 3.1-kb Eco RI-Bam HI and 1.34-kb Bam HI-Xba I fragments were ligated after the Eco RI and Xba I overhangs were blunt-ended by Klenow treatment.

The vector pMD-MLVogp was used, (Harvard Medical School, SEQ ID NO:29, 9633 bp, helper plasmid for murine leukemia virus (MLV) retroviral vectors (encodes MLV gag-pol polyproteins, has human beta-globin intron and polyA signal; amp resistance).

For VNP (virus-like nanoparticle) production, 3×10⁶ HEK-293T cells were seeded onto 150-mm culture dishes, transfected the day after with 30 μg of MoMLV original gag-pol (OGP) plasmid (Construct, see FIG. 9) and 60 μg of pK12::FLAG::RBD::GPI. VNP-containing supernatants were harvested after 72 hours, filtered (0.45 μm, Millipore, Billerica, MA), concentrated by ultrafiltration (Centricon Plus-70, Merck Millipore Ltd., Tullagreen, Ireland), and followed by further concentration by ultracentrifugation using a SW41 Ti rotor (1×10⁵ g, 1 hour, Beckman-Optima LE-80K, Beckman Instruments, Palo Alto, CA). Protein concentrations of PBS-washed VNP preparations were determined (Micro BCA, Thermo Fisher, Waltham, MA) and adjusted. VNP were stored at 4° C. until use for up to 4 weeks, without alteration of biological activity. (Ref. 43).

SDS-PAGE was carried out with 10 μg of purified VNP samples per lane, lysed with 4×SDS-PAGE loading dye (40% glycerin, 200 mM Tris, 4% SDS, 0.04% bromphenole blue) supplemented with 300 mM DTT for reducing conditions or without DTT for non-reducing conditions and resolved on 4-20% polyacrylamide gels. Afterwards, proteins were transferred onto a PVDF membrane by semi-dry blotting technology (Peqlab Biotechnology, Erlangen, Germany) and the membranes were blocked with Tris buffered saline (50 mM Tris, 150 mM NaCl) containing 0.05% tween-20 (Biorad Laboratories, Hercules, CA, USA) and 5% non-fat dry milk (Maresi Austria, Vienna, Austria) (TBS-T) and incubated with sera of a COVID-19 convalescent and a healthy control individual as primary antibodies overnight at 4° C. After 5 times careful washing with TBS-T, membranes were incubated for 1 hour at room temperature with goat-anti-human IgG HRP-conjugated Fab's (Jackson Immunotechnology) and after extensive washing, blots were developed with a luminol-based indicator system (Biorad Laboratories, Hercules, CA, USA) and photographs were taken with the chemiluminescent imaging system LAS-4000 (GE Healthcare).

For the proliferation of PBMC from COVID-19 convalescent patients and healthy control individuals, 1×10⁵ PBMC were incubated with purified SARS-CoV-2 antigen-expressing VNP (5 μg/ml) expressing the GPI anchored antigens (FLAG::RBD::GPI from SARS-CoV-2, FLAG::S::GPI from SARS-CoV-2, FLAG::NC::GPI from SARS-CoV-2), empty VNP (5 μg/ml), FSME antigen (0.15 μg/ml), Tetanus toxoid (0.0125 IE/ml), medium alone or PHA (12.5 μg/ml) in total of 200 μl of AIMV Medium (Thermo Fisher) plus 2% of human serum (Octapharma, Vienna, Austria) per well round bottom 96 well plates (Sarstedt AG, Nümbrecht, Germany). All conditions were set up in triplicates and incubated for 144 h followed by a 18 hours methyl-[3H]-thymidine puls (1 μCi/well). After this incubation time, T cell proliferation was quantified on a Betaplate Counter (Perkin Elmer, Waltham, MA).

### Results:

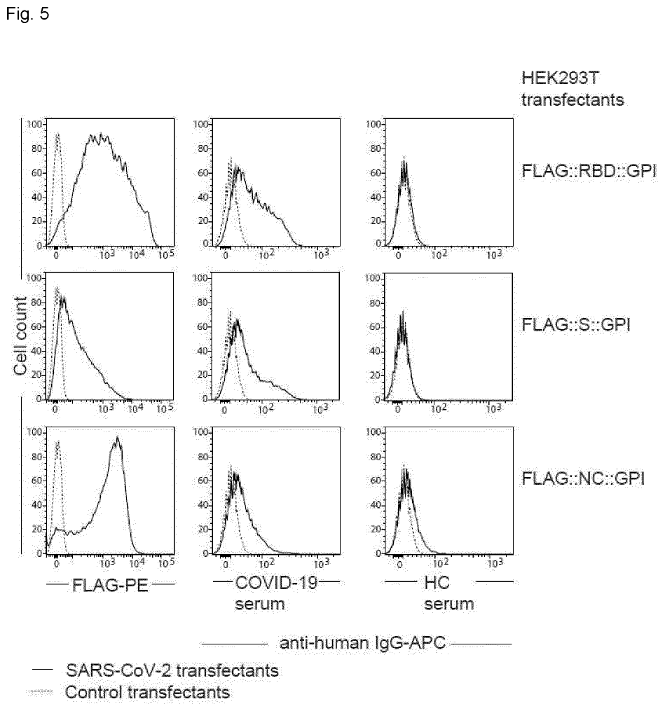

As a first step, it was confirmed that the SARS CoV-2 RBD construct (FLAG::RBD::GPI) is well expressed in HEK293T VNP producer cells. To this end, HEK293T cells were transiently transfected with pEAK12 SARS CoV-2 RBD using the calcium phosphate precipitation method (Ref. 44) and RBD expression was verified after 72 hours by two different methods. Firstly, by reactivity with an anti-FLAG tag antibody, and secondly, with serum derived from a COVID-19 convalescent subject. FIG. 5 shows that, indeed, a large proportion of SARS CoV-2 RBD HEK293T cells stained positive with the anti-FLAG-tag antibody (solid line; 91.7% positive) which is directed against the N-terminal triple FLAG-tag sequence (Ref. 45). The dotted line represents fluorescence obtained with unstained HEK293T cells, indicating the cellular background fluorescence. The clear anti-FLAG antibody reactivity already indicated that the FLAG::RBD::GPI fusion protein is cell surface expressed on a large proportion of transfectants, as expected. Similarly, a 1:100 dilution of serum from a COVID-19 convalescent patient positively stained a large fraction of transfectants when counterstained with goat-anti-human IgG (gamma chain specific)-APC conjugated Fab's, demonstrating the immunoreactivity and thus the proper folding of the RBD domain as it appears by cell surface expression of the FLAG::RBD::GPI fusion protein. In marked contrast, a 1:100 dilution of serum from a subject who never had COVID-19 did not react with the FLAG::RBD::GPI transfectants, i.e., staining with this serum was comparable to unstained cells, clearly indicating the specificity of the staining pattern with COVID-19 convalescent serum.

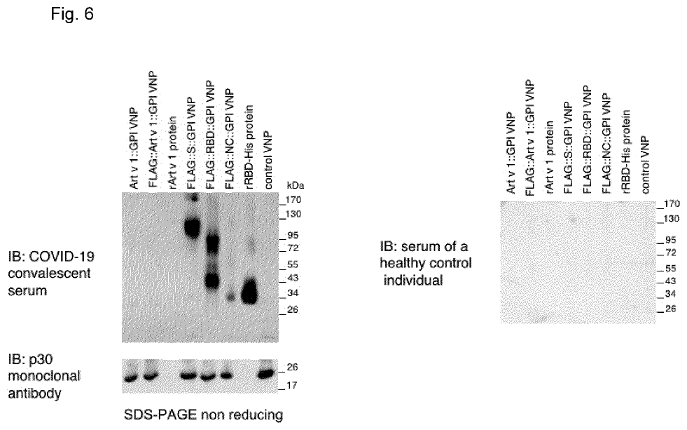

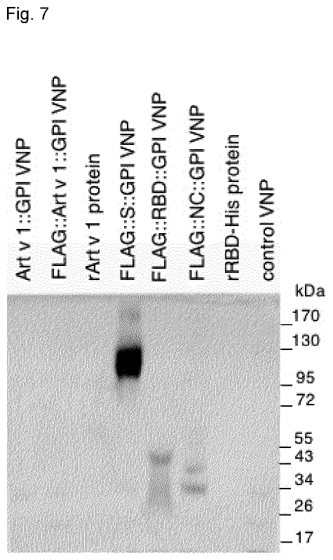

In a next step, VNP budding was induced in HEK293T cells transfected with FLAG::RBD::GPI to investigate whether VNP would be efficiently decorated with the FLAG::RBD::GPI fusion protein. To this end, HEK293T cells were transiently transfected with MoMLV gag-pol encoding Moloney core protein and co-expressed FLAG::RBD::GPI in parallel. After three days, VNPs secreted into the supernatant of these HEK293T cells were isolated and analyzed for the presence of immuno-reactive, correctly folded RBD protein by SDS-PAGE followed by Western blotting with serum from a COVID-19 convalescent and a healthy control subject under nonreducing (FIG. 6) and reducing conditions (FIG. 7). In parallel, VNP expressing the FLAG::S::GPI, FLAG::NC::GPI, Art v 1::GOI, FLAG::Art v 1::GPI, empty VNP or purified recombinant RBD-His protein, were analyzed. In FIG. 6, it was found that FLAG::RBD::GPI was clearly detected as 40 kDa and 95 kDa bands by serum from a COVID-19 convalescent, but not by serum from a healthy control subject. Interestingly, the RBD reactivity of the convalescent serum disappeared when the VNP lysate was separated under reducing conditions in the presence of dithiothreitol (DTT) (FIG. 7). These results highlight the following. First, FLAG::RBD::GPI can successfully decorate MoMLV-derived VNP. Second, the FLAG::RBD::GPI fusion protein is present in an immuno-reactive form on the surface of VNP, it is recognized by the polyclonal serum of a COVID-19 convalescent. Third, reduction of disulfide bonds in the FLAG::RBD::GPI fusion protein using DTT alters the conformation of FLAG::RBD::GPI such that it is no longer recognized by the serum of the COVID-19 convalescent subject. Fourth, even when FLAG::RBD::GPI is expressed on VNP in the correct conformation (non-reducing conditions), it is not recognized by the serum of a healthy control subject. Fifth, the immunoreactivity of the convalescent serum is specific in that it recognizes only VNP expressing FLAG::RBD::GPI fusion protein, but not VNP that have been decorated with other unrelated proteins on their surface, such as FLAG::Art v 1::GPI, or that have remained undecorated.

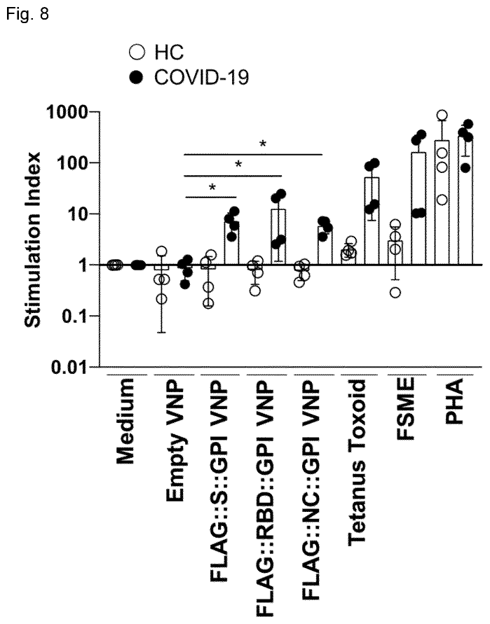

FIG. 8 shows that the VNP-borne FLAG::RBD::GPI is also recognized by T lymphocytes from COVID-19 convalescent subjects, but not by T cells from healthy control subjects. In the present T cell stimulation experiments, T cells from COVID-19 convalescent patients and from healthy control subjects were co-incubated for 6 days with 5 μg VNP decorated with the indicated fusion proteins or left undecorated. After six days of culture, T cells were pulsed with methyl-³H-thymidine overnight and harvested the next day, and the degree of methyl-³H-thymidine incorporation (radioactivity) into their newly synthesized DNA was taken as a measure of cellular proliferation. FIG. 8 shows that VNP decorated with FLAG::RBD::GPI significantly stimulated T cell proliferation from convalescent but not healthy control subjects. The stimulation indices ranged from 2.5 to 24.6 PHA was used as a positive control, resulting in stimulation indices of 340.3 fold. Admittedly, the degree of polyclonal T cell activation induced by PHA was significantly more pronounced. Nevertheless, VNP decorated with FLAG::RBD::GPI stimulated T cells with a mean stimulation index of 12.7±18.0. Similar results were obtained with S protein-decorated VNP and with NC protein-decorated particles (stimulation indices 7.2±7.2- and 5.8±3.7-fold). These results suggest that the FLAG::RBD::GPI fusion protein can also be taken up by antigen-presenting cells and presented in a immunogenic form to T cells, which subsequently leads to their significant activation. While T cell stimulation by immunogenic peptides derived from foreign proteins is certainly not conformation dependent, the experiments prove the presence on VNP of proteins immuno-reactive to T cells of COVID-19 convalescent but not healthy control individuals, and thus specificity of the expression system on the T cellular level.

### REFERENCES OF EXAMPLE 6

40. Hopp, T. P. et al. A Short Polypeptide Marker Sequence Useful for Recombinant Protein Identification and Purification. Nat. Biotechnology. 6, 1204-1210, doi:10.1038/nbt1088-1204 (1988)
41. Derdak, S. V. et al. Direct stimulation of T lymphocytes by immunosomes: virus-like particles decorated with T cell receptor/CD3 ligands plus costimulatory molecules. Proc Natl Acad Sci USA 103: 13144-13149. doi: 10.1073/pnas.0602283103. (2006)
42. Simmons, D., and B. Seed. 1988. The Fc gamma receptor of natural killer cells is a phospholipid-linked membrane protein. Nature 333: 568-570. doi: 10.1038/333568a0. (1988)
43. Kratzer, B., et al. Prevention of allergy by virus-like nanoparticles (VNP) delivering shielded versions of major allergens in a humanized murine allergy model. Allergy 74(2): 246-260. doi: 10.1111/all.13573. (2019)
44. Jordan, M., A. Schallhorn, and F. M. Wurm. Transfecting mammalian cells: optimization of critical parameters affecting calcium-phosphate precipitate formation. Nucleic Acids Res 24: 596-601. doi: 10.1093/nar/24.4.596. (1996)
45. Park, S. H., et al. Generation and application of new rat monoclonal antibodies against synthetic FLAG and OLLAS tags for improved immunodetection. J Immunol Methods 331: 27-38. doi: 10.1016/j.jim.2007.10.012 (2008)
46. Ory, D. S., B. A. Neugeboren, and R. C. Mulligan. A stable human-derived packaging cell line for production of high titer retrovirus/vesicular stomatitis virus G pseudotypes. Proc Natl Acad Sci USA 93: 11400-11406. doi: 10.1073/pnas.93.21.11400 (1996)
47. Danos, O., and R. C. Mulligan. Safe and efficient generation of recombinant retroviruses with amphotropic and ecotropic host ranges. Proc Natl Acad Sci USA 85: 6460-6464. doi: 10.1073/pnas.85.17.6460 (1988)

### Example 7: Characterization of Recombinant SARS-CoV-2 Subunit Vaccines

A fusion protein (PreS-RBD) consisting of two RBD domains, one fused to the N-terminus and one fused to the C-terminus of the human hepatitis virus B (HBV)-derived PreS was prepared as described above (SEQ ID NO:14, including the His-tag; SEQ ID NO:100 without the His-tag), (FIG. 10a). Synthetic genes coding for PreS-RBD and, for control purposes for RBD alone (SEQ ID NO:1), were codon-optimized either for the expression in E. coli or in human cell lines. Furthermore, RBD-fusion proteins consisting of two linked RBDs (RBD Dimer, SEQ ID NO:15) or three linked RBDs (RBD Trimer, SEQ ID NO:16) as well as a fusion protein consisting of two RBD domains, one fused to the N-terminus and one fused to the C-terminus of the SARS-CoV-2 nucleocapsid protein (N-RBD, SEQ ID NO:99) were designed an expressed in HEK cells. The N-RBD fusion protein was designed as a fusion of two RBD (aa330-aa522, SARS-CoV-2 Genbank accession No.: QHD43416.1) linked to the N- and C-terminus of the SARS-CoV-2 nucleocapsid protein (SARS-CoV-2 Genbank accession Nr.: QHD43416.1). The synthetic DNA molecules were codon optimized for the expression in HEK cells and contained a 5′ DNA coding for a N-terminal IL-2 signal peptide (MYRMQLLSCIALSLALVTNS, SEQ ID NO:101) and a 3′ DNA coding for a C-terminal hexahistidine tag. Expressed proteins were purified via Nickel affinity chromatography via a hexahistidine tag which was added to the recombinant proteins. It is understood that each of the described vaccine antigens can be made also without the His-tag. E. coli expressed PreS-RBD migrated at approximately 60 kDa in SDS-PAGE under reducing and non-reducing conditions whereas the HEK cell-expressed fusion protein migrated at 70 kDa (FIG. 10b). The higher molecular weight of HEK cell-expressed PreS-RBD versus E. coli-expressed PreS-RBD is compatible with the presence of six N-glycosylation sites in the former protein. Likewise, HEK cell-expressed RBD containing two N-glycosylation sites had a higher molecular weight (i.e., 35 kDa) compared to E. coli-expressed RBD (i.e., 32 kDa). E. coli-expressed RBD also showed additional bands under reducing and non-reducing conditions (FIG. 10b) which stained with anti-His antibodies and hence did not represent impurities (data not shown).

The analysis of recombinant RBD proteins regarding the presence of fold and secondary structure by far UV circular dichroism spectroscopy (CD) analyses is presented in FIG. 10c. RBD expressed in HEK cells revealed a minimum at 207 nm, which is consistent with a previous study reporting the expression of functional RBD 4 resembling a predominant β-sheet structure. HEK cell-expressed PreS-RBD exhibited a minimum at 209 nm which is also indicative of the presence of considerable β-sheet secondary structure (FIG. 10c). E. coli-expressed RBD and PreS-RBD showed a strong reduction of ellipticity and of the corresponding minima indicating the presence of a high proportion of unfolded structure in the proteins (FIG. 10c).

In a next set of experiments, recombinant RBD and PreS-RBD proteins were characterized regarding their reactivity with a panel of antibody probes specific for PreS, RBD and the His-tag (FIG. 10d-g). FIG. 10d shows that E. coli- and HEK cell-expressed RBD and PreS-RBD reacted with different dilutions of anti-His antibodies (HEK cell-expressed PreS-RBD and RBD >E. coli-expressed PreS-RBD and RBD). No reaction was observed when the primary anti-His antibody was omitted (FIG. 10d). HEK cell->E. coli-expressed PreS-RBD reacted with antisera raised against PreS-peptides and against E. coli-expressed PreS whereas recombinant RBD proteins did not (FIG. 10e,f). No reaction was observed when the primary anti-PreS antibodies were omitted (FIG. 10e,f). Next, sera obtained from subjects before the SARS-CoV-2 pandemic (i.e., historic control sera) and sera obtained from COVID-19 convalescent patients were tested for IgG reactivity with E. coli- and HEK cell-expressed RBD and PreS-RBD proteins. Historic control sera showed no IgG reactivity to folded RBD and PreS-RBD, whereas a few sera (i.e., P003, P004, P010) showed low reactivity to unfolded RBD and PreS-RBD (FIG. 10g, left). By contrast, sera from COVID-19 convalescent patients showed pronounced IgG reactivity to HEK cell-expressed PreS-RBD>RBD but no relevant reactivity to E. coli-expressed proteins (FIG. 10g, right). Only few sera (i.e., B013, I002) showed very weak reactivity to the unfolded bacterially expressed proteins. No reactivity was observed when patients' sera were omitted (FIG. 10g).

### Induction of RBD-Specific Antibody Response

The ability of folded PreS-RBD, RBD, RBD Dimer, RBD Trimer or N-RBD to induce antibody responses was investigated by immunizing rabbits, which allows studying the uniformity of the induced immune responses in out-bred animals and thus to identify poor- or no-responders. The choice of out-bred animals is important because it was found that approximately 20% of SARS-CoV-2-infected subjects did not mount RBD-specific antibodies and hence represented “RBD-non-responders”. Groups of three rabbits were immunized three times in three-week intervals with 20 or 40 or 80 μg of alum-adsorbed RBD or two doses of alum-adsorbed PreS-RBD, RBD Dimer, RBD Trimer or N-RBD containing equimolar amounts of RBD. FIG. 11 shows RBD-specific IgG levels measured by ELISA for three different dilutions of sera of the rabbits. Even at day 42 three rabbits (rabbits #3, #5, #6) out of the six RBD only-immunized animals were non- and low-responders (OD values <0.5) at a dilution of 1:1000, two rabbits (rabbits #11, #14) out of the six RBD-dimer immunized animals were non-responders and two rabbits (rabbits #21, #24) out of the six RBD-trimer immunized animals were non-responders according to the same definition. By contrast, each of the six PreS-RBD-immunized rabbits with 20 or 40 μg developed robust and uniform RBD-specific IgG levels already at day 35 (OD values > or =0.5 at a 1:1000 serum dilution) increasing to day 42 (FIG. 11). Immunization with 20 and 80 μg N-RBD led to robust RBD-specific IgG levels in 83.3% of immunized animals at day 35 increasing to 100% at day 43 (OD values > or =0.5 at a 1:1000 serum dilution). (FIG. 11).

Immunization with Folded but not with Unfolded PreS-RBD Induces Antibodies in Serum of a COVID-19 Naïve Subject that Cross-React with SARS-CoV-2 Variants

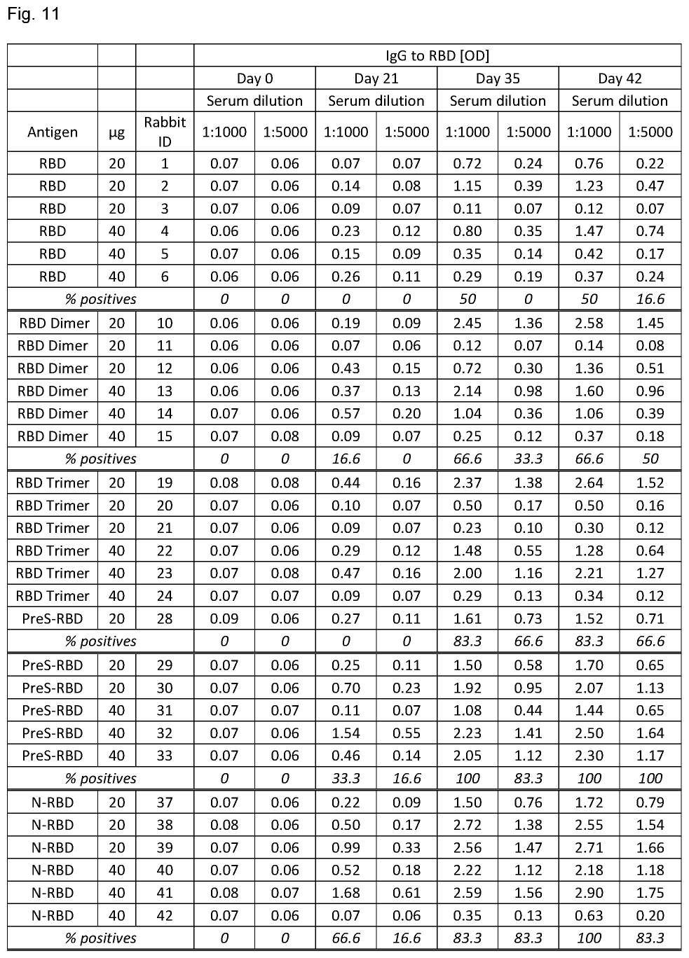

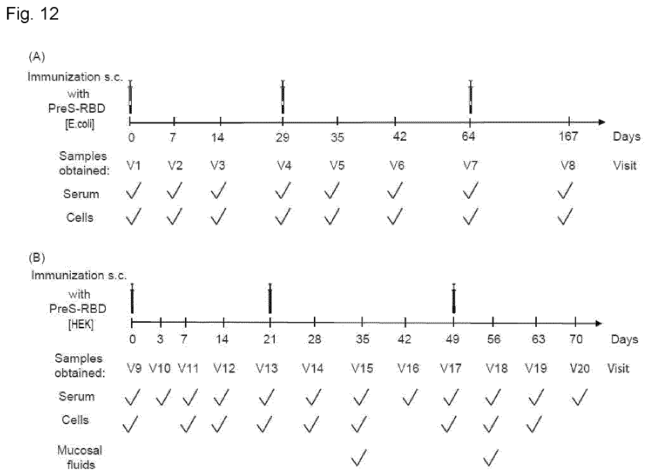

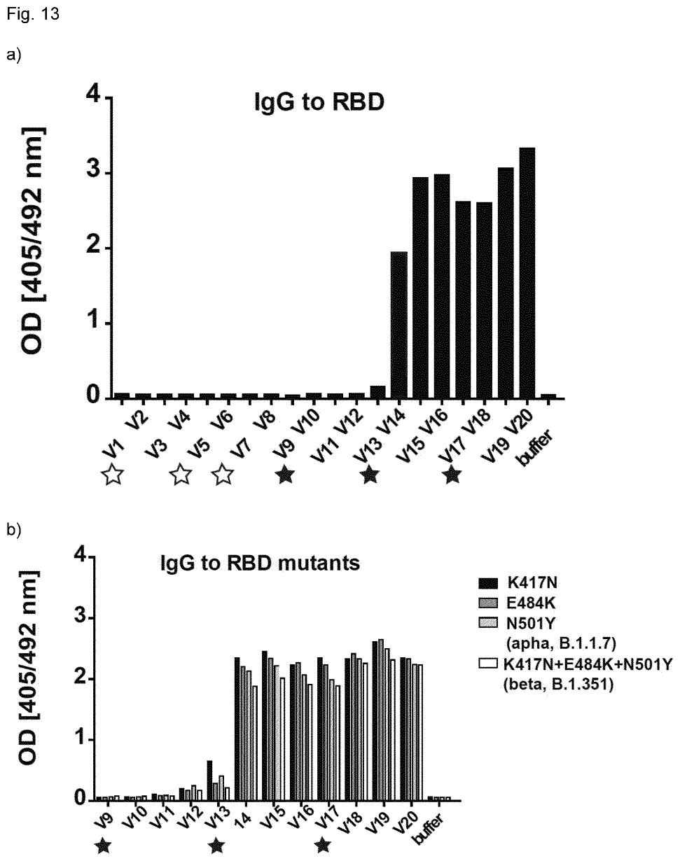

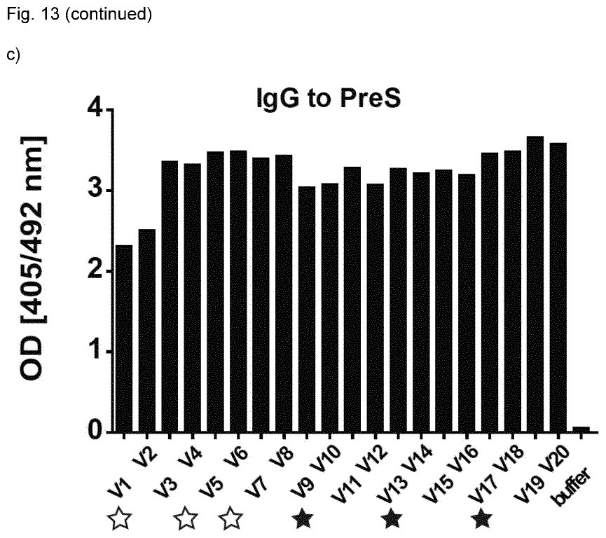

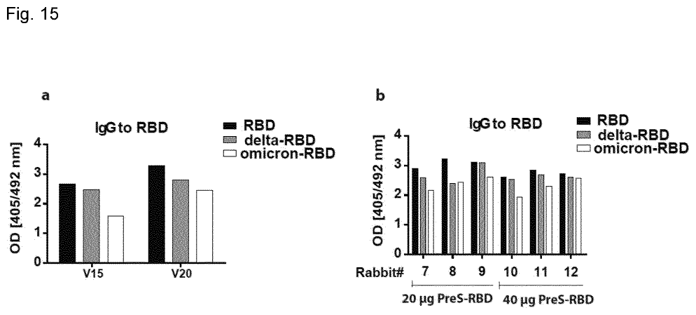

Immunization of a SARS-CoV-2 naïve human subject was first with unfolded E. coli-expressed PreS-RBD (FIG. 12). In total, three subcutaneous injections were administered approximately 4 weeks apart. FIG. 13a shows that immunization with unfolded PreS-RBD did not induce IgG responses against folded HEK cell-expressed RBD. This result was in agreement with data obtained in rabbits where E. coli-expressed PreS-RBD failed to induce IgG responses against folded RBD. These results and the finding that only folded RBD induced IgG antibodies against folded RBD in rabbits, which strongly neutralized SARS-CoV-2 infections in vitro and prevented RBD binding to ACE2 led to the construction of a recombinant PreS-RBD which was completely identical in its primary sequence as the E. coli-expressed version but due to expression in HEK cells was obtained as folded protein (FIG. 10). Immunization of the human subject with folded, HEK cell-expressed PreS-RBD was later initiated in the subject and induced a strong IgG response against folded RBD, as determined one week after the second injection (i.e., at visit 14) (FIG. 12, FIG. 13a). Moreover, the RBD-specific antibodies induced by folded PreS-RBD based on the Wuhan Hu-1 sequence induced IgG antibodies which cross-reacted equally with SARS-CoV-2 variants (Wuhan, K417N, E484K, Alpha, Beta, Delta, Omicron) (FIG. 13a,b, FIG. 15). Of note, also rabbit antibodies induced by immunization with the folded HEK cell-expressed Wuhan PreS-RBD protein cross-reacted with SARS-CoV-variants delta and omicron (FIG. 15).

Since the volunteer had been previously vaccinated with the PreS-containing grass pollen allergy vaccine BM325 (i.e., VVX001) (ClinicalTrials.gov Identifier: NCT03625934), a PreS-specific IgG response was detected as early as visit 1, which further increased during immunization with E. coli-expressed PreS-RBD and even more so after immunization with folded HEK cell-expressed PreS-RBD (FIG. 13c). Further analysis shows that the IgG isotype dominated the RBD-specific antibody responses in the immunized subject and was accompanied by a low IgM response and an IgA response which peaked shortly after the beginning of the immunization (data not shown). The PreS-specific antibody response in the subject was dominated by IgG antibodies, some IgM responses but no relevant IgA response (data not shown).

Immunization with Folded PreS-RBD Induces Antibodies Reacting with the NTCP Binding Sites of HBV Genotypes A-H

The PreS protein contains at its N-terminus the binding site of HBV to its receptor NTCP on hepatocytes, and, therefore, is a candidate vaccine antigen for preventive and therapeutic HBV vaccines. Due to previous vaccination with BM325, a component of BM32, the subject has had IgG antibodies specific for PreS-derived peptides, in particular to PreS P2 which contains the NTCP binding site of HBV and against peptides including the amino acid sequence crucial for infectivity (PreS aa13-aa51) of HBV genotypes A-H. Vaccination with three doses of unfolded E. coli-expressed PreS-RBD increased the PreS peptide-specific IgG responses at visit 9 (FIG. 12) as determined approximately half a year after the last vaccination with E. coli-expressed PreS-RBD (i.e., at visit 7). The administration of three doses of folded HEK cell-expressed PreS-RBD strongly increased IgG levels to peptides spanning PreS, in particular the N-terminal peptides containing the NTCP binding site and peptides representing the NTCP binding sites from all 8 HBV genotypes as measured at visit 20, approximately four weeks after the third injection (FIG. 12).

Immunization with Folded PreS-RBD Induces an Early RBD-Specific IgG₁ response followed by a late but sustained IgG₄ Response

It was previously found that immunization with the PreS-containing grass pollen allergy vaccine BM32 induces a bi-phasic allergen- and PreS-specific IgG response which consists of an early IgG₁ followed by a late but sustained IgG₄ subclass response. The late and sustained IgG₄ response is considered to be responsible for the long-term protective effect of allergen-specific immunotherapy which persists for several years even after discontinuation of vaccination.

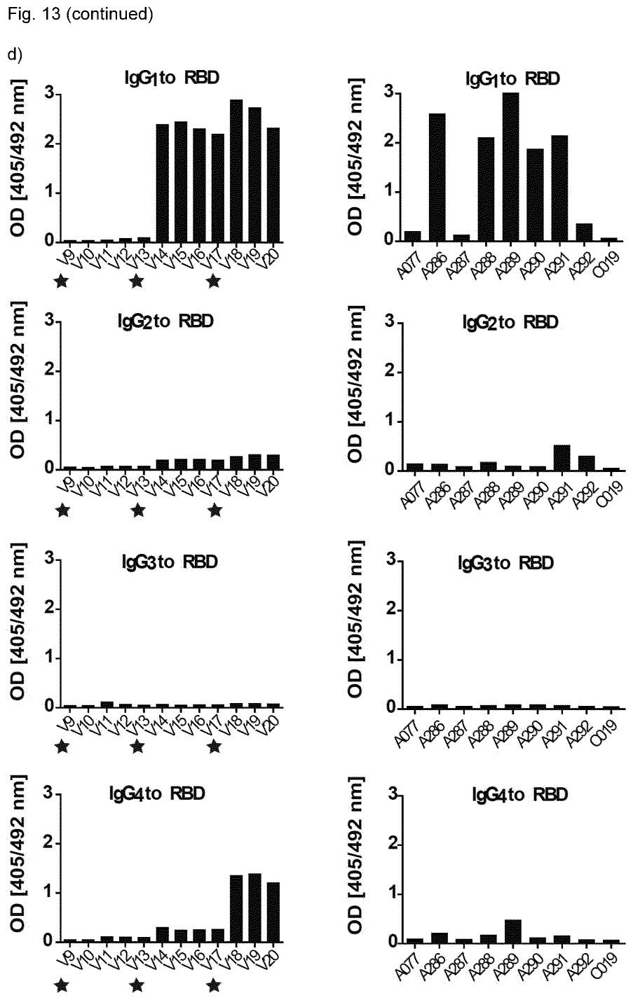

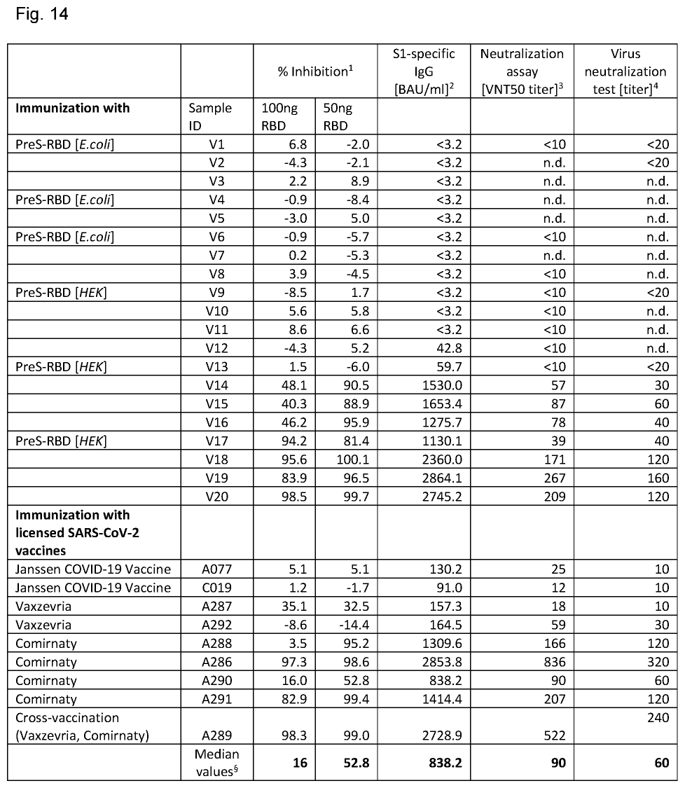

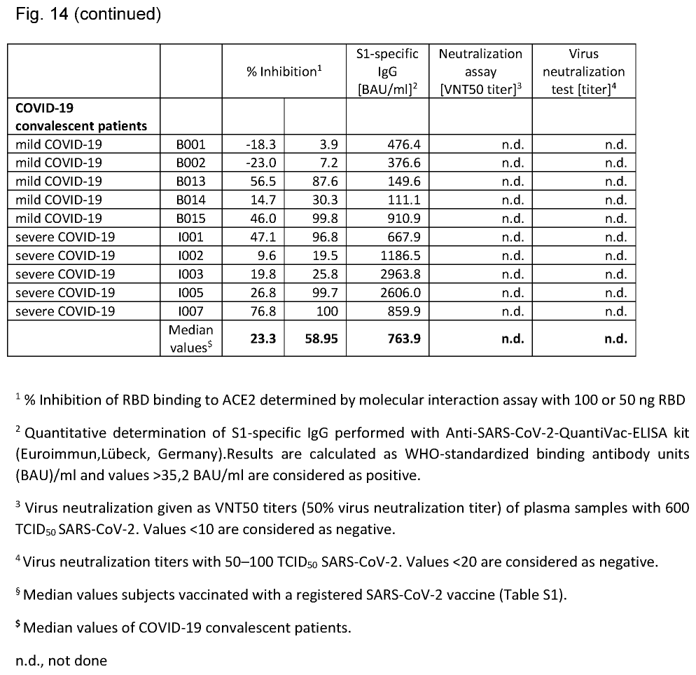

FIG. 13d shows the development of RBD-specific IgG subclass responses in the subject after immunization with folded HEK cell-expressed PreS-RBD. A strong IgG₁ subclass response to folded RBD response was observed already at visit 14 after the second vaccination whereas folded RBD-specific IgG₄ antibodies increased only later, i.e., after the third vaccination. Low RBD-specific IgG₂ levels and no RBD-specific IgG₃ responses were found (FIG. 13d). In parallel, S and RBD-specific IgG responses in randomly enrolled healthy subjects were analyzed approximately four weeks after full vaccination with SARS-CoV-2 vaccines registered in Europe (i.e., Janssen COVID-19 vaccine, Johnson&Johnson; Vaxzevria, AstraZeneca; Comirnaty, BionTech/Pfizer) (FIG. 14). Four out of the nine randomly enrolled healthy vaccinated subjects (i.e., A287, A292, A077, C019) mounted only low S- and almost no RBD-specific IgG responses. The quantification of S1-specific antibody levels confirmed these results, showing that they had S1-specific antibody levels below 200 BAU/ml (Table 5, see FIG. 14) which were lower than those of the majority (i.e., eight out of ten) of COVID-19 convalescent subjects (FIG. 14). The RBD-specific IgG subclass response in subjects vaccinated with registered vaccines consisted mainly of an IgG₁ subclass response, little IgG₂, almost no IgG₄ and no IgG₃ (FIG. 13d).

### Antibodies in Serum, Tears and Nasal Secretions of the PreS-RBD Immunized Subject Recognize Exclusively Conformational RBD Epitopes

A detailed analysis of the SARS-CoV-2-specific antibody responses was performed in the immunized subject using a solid-phase chip containing a large panel of micro-arrayed SARS-CoV-2 proteins as well as peptides spanning the S protein during the entire period of immunization with folded and unfolded PreS-RBD (V1-V20). Immunization with unfolded E. coli-expressed PreS-RBD only induced antibodies against the immunogen (i.e., unfolded E. coli-expressed PreS-RBD) but no SARS-CoV-2-specific IgG, IgA or IgM antibody response. Immunization with folded HEK cell-expressed PreS-RBD induced a strong and sustained IgG response against folded RBD and against proteins containing folded RBD (i.e., insect cell-expressed S and S1, HEK cell-expressed S1) whereas no IgG responses to sequential RBD-derived peptide epitopes were detected. The RBD-specific IgG response was accompanied by an initially strong but only transient IgA response specific for folded RBD. No relevant SARS-CoV-2-specific IgM responses were found throughout the immunization period. Immunization with folded PreS-RBD boosted the IgG response against unfolded PreS-RBD which is attributable to PreS-specific IgG antibodies. Of note, no antibodies specific for the nucleocapsid protein (NP) or for S2 lacking RBD were observed during the whole period of immunization and observation. It was found that high levels of IgG antibodies specific for folded RBD and to a lower extent to E. coli-expressed unfolded PreS-RBD were also present in nasal secretions and tears obtained at visits 15 and 18. At these time points also moderate levels of IgA antibodies specific for folded RBD could be detected whereas no SARS-CoV-2-specific IgM antibodies were found.

Antibodies Induced by Immunization of the Subject with Folded PreS-RBD Inhibit Binding of RBD to ACE2 and Neutralize SARS-CoV-2

FIG. 14 provides an overview of the development of S1-specific IgG antibodies, of antibodies inhibiting the binding of RBD to ACE2 and of virus-neutralizing antibodies in the immunized subject. Sera obtained from nine healthy subjects approximately four weeks (median=27 days) after full vaccination with licensed genetic COVID-19 vaccines and sera from ten COVID-19 convalescent patients obtained approximately 8 weeks after SARS-CoV-2 infection were included in the assays for comparison. Immunization with unfolded E. coli-expressed PreS-RBD neither induced S1-specific IgG antibodies nor antibodies inhibiting the interaction of RBD and ACE and also no virus-neutralizing antibodies were detected (FIG. 14, visits 1-9). At visits 19 and 20 (i.e., 3 and 4 weeks after the third immunization), S1-specific IgG antibody levels exceeded 2700 BAU/ml in the PreS-RBD-immunized subject and were higher than the median S1-specific IgG antibodies in subjects vaccinated with licensed vaccines (i.e., 91.0-2853.8 BAU/ml; median: 838.2 BAU/ml) and in COVID-19 convalescent subjects (i.e., 111.1-2963.8 BAU/ml; median: 763.9 BAU/ml). Serum obtained from the immunized subject at visit 20 inhibited the binding of 100 ng and 50 ng RBD to ACE2 by more than 98% whereas median inhibitions obtained with sera from subjects vaccinated with licensed genetic vaccines (100 ng RBD: −8.6-98.3% inhibition, median inhibition: 16.0%; 50 ng RBD: −14.4-99.4% inhibition, median inhibition: 52.8%) and sera from COVID-19 convalescent subjects were much lower. In the virus neutralization assay using 600 TCID₅₀ authentic SARS-CoV-2 (BetaCoV/Munich/BavPat1/2020 isolate), the VNT50 titer (which indicates the reciprocal serum dilution yielding a 50% reduction in anti-SARS-CoV-2 NP staining of the infected Vero cells measured by ELISA two days later) of the PreS-RBD-immunized subject at visit 19 and 20 was 267 and 209, respectively, was higher than the median VNT50 titers (i.e., 12-839; median: 90) found for subjects vaccinated with licensed vaccines. These results are in agreement with the data obtained by the virus neutralization test using 50-100 TCID₅₀ SARS-CoV-2, which expresses the VNT as reciprocal serum dilution required for 100% protection against virus-induced cytopathic effects. The VNTs in this assay obtained for the PreS-RBD-vaccinated subject at visits 19 and 20 were 160 and 120, respectively, and thus also higher than the median VNT (10-320; median: 60) as determined in subjects vaccinated with licensed vaccines.

As exemplified for a recombinant PreS-RBD fusion protein also the other herein described subunit vaccines can be produced in large quantities and high purity through expression in mammalian cells such as HEK cells which is a process that is well established all over the world not only for vaccines but also for the production of vaccines and biologics. It could be demonstrated that the immunogen/antigen as a structurally folded protein was beneficial because immunization with unfolded PreS-RBD failed to induce RBD-specific antibodies that are necessary to inhibit the RBD-ACE2 interaction and to achieve virus neutralization. It was shown that the determination of the structural fold of PreS-RBD can be performed by using biophysical methods such as circular dichroism (CD) spectroscopy analysis of the protein and/or by showing the reactivity of the recombinant antigen with IgG antibodies from COVID-19 convalescent patients which specifically react with the folded but not with the unfolded, E. coli-expressed PreS-RBD (FIG. 10c,g).

PreS-RBD is a recombinant protein and thus it is possible to perform precise dose-finding studies to determine the optimal amount of the immunogen for vaccination which is not possible for genetic vaccines. The recombinant PreS-RBD was formulated by adsorption to aluminum hydroxide, an adjuvant which has been safely used in numerous vaccines for decades. In the present pilot stability studies, it was found that approximately 90% of PreS-RBD is bound to aluminum hydroxide and thus the injected antigen remains to a large extent at the injection site (Gattinger and Valenta, unpublished). Aluminum hydroxide-formulated PreS-RBD remains stable for months at +4° C. and also storage at higher temperature does not seem to affect the stability and immunogenicity of the vaccine which is an advantage for a vaccine to be distributed and used globally, especially in countries with low resources (data not shown).

The present study indicates that administration of two to three doses of a molar equivalent of 40 microgram of folded PreS-RBD induces a robust induction of RBD-specific antibody responses, which are accompanied by specific T cell responses and induction of B memory/plasma cell responses. Results obtained in the herein immunized subject demonstrate that the RBD-specific antibody response consists mainly of an IgG response composed of an early IgG₁ and late IgG₄ response of which the latter was not observed with genetic COVID-19 vaccines (FIG. 13d) so far. The biphasic induction of RBD-specific (i.e., early IgG₁ and late, sustained IgG₄) is very similar to that of BM32, a therapeutic grass pollen allergy vaccine which contains recombinant fusion proteins consisting of PreS and allergen peptides (Eckl-Dorna, J., et al. EBioMedicine. 2019. 50, 421-432). BM32 has been safely used for the treatment of grass pollen induced allergy in several clinical studies (ClinicalTrials.gov Identifier: NCT02643641) and it has been shown that BM32-induced PreS-specific antibodies protect against HBV infections in vitro because they are directed against the N-terminal part of PreS containing the binding site of HBV for the NTCP receptor on human hepatocytes (Cornelius, C., et al., EBioMedicine. 2016.11, 58-67; Tulaeva, I., et al., EBioMedicine. 2020. 59, 102953). In fact, very recently, it has been shown that immunization of patients with chronic HBV infections with BM325 (i.e., VVX001) had induced HBV-neutralizing antibodies in vivo ( )⁴³. Furthermore, PreS-RBD not only induced RBD-specific IgG antibodies but also PreS-specific antibodies reacting with the NTCP binding sites of HBV genotypes A-H and hence may protect also against HBV infections (FIG. 13c). However, the exemplary PreS-RBD fusion protein was made not only with the intention to induce SARS-CoV-2- and HBV-neutralizing antibodies but to use PreS also as a carrier protein to enhance the immunogenicity of RBD. In a previous study, it was found that approximately 20% of SARS-CoV-2-infected patients did not produce RBD-specific IgG antibodies (Gattinger, P., et al., Allergy. 2021. 76, 878-883) RBD-specific antibodies contribute to the induction of sterilizing immunity to SARS-CoV-2 because these antibodies prevent the virus from binding to its receptor ACE2 on human cells and thus can be critically important for virus neutralization (Gattinger, P., et al., Allergy. 2021. 76, 878-883). It was therefore hypothesized that immunization with RBD alone will eventually not be sufficient to induce uniform and robust RBD-specific antibodies in an outbred population. Indeed, the hypothesis was supported by results obtained from the immunization of outbred rabbits with RBD, RBD Dimer, RBD Trimer and the PreS-RBD or N-RBD fusion proteins. In this study it was found that approximately 20-30% of rabbits failed to produce robust RBD-specific antibodies when they were immunized with 20 or 40 μg RBD, RBD Dimer or RBD-Trimer whereas all rabbits immunized with 20 or 40 μg PreS-RBD produced uniform and robust RBD-specific antibodies, which could also be observed after immunization with 20 or 80 μg N-RBD. This result can be explained by the hapten-carrier principle of covalent coupling or fusion of a less immunogenic component (i.e., the hapten) to a protein carrier which can enhance the immunogenicity of the hapten (Paul, W. E., et al., J Exp Med. 1970.132, 283-299). This principle was extensively used for the construction of allergy vaccines based on allergen-derived peptides fused to PreS to enhance the immunogenicity of the allergen peptides (Valenta, R., et al. Immunol Lett. 2017. 189, 19-26). Thus, the results obtained in this study are in agreement with previous work performed in AIT.

The RBD-specific IgG antibodies induced in the human subject with PreS-RBD cross-reacted with RBD mutants and variants including even the highly mutated VOC omicron (FIG. 13b) suggesting that the PreS-RBD-based vaccine has the potential to cross-protect even against strongly mutated VOCs. PreS-RBD contains two RBD domains, one fused to the N- and one fused to the C-terminus of PreS. The cross-protective effect can even be enhanced by including RBDs from the two most divergent and most common SARS-CoV-2 VOCs in the PreS-RBD construct. This would have the advantage that the relevant epitopes of two SARS-CoV-2 VOCs can be included in only one antigen, which will allow addressing the challenge of emerging virus variants in a highly effective manner.

The RBD-specific antibodies induced in the PreS-RBD-immunized subject were found to block more strongly the binding of RBD to ACE2 than those obtained from subjects after full vaccination with currently available and licensed COVID-19 vaccines and from COVID-19 convalescent patients when determined by their median blocking activity (FIG. 14). These results were confirmed by testing the VNTs using two different virus neutralization assays, one measuring the production of virus antigen and the second determining the virus cytopathic effect.

In addition to the fact that folded PreS-RBD induces antibodies which block RBD-ACE2 binding and thus infection of the host cell, also other observations indicate, the folded PreS-RBD has features of a vaccine which could be used to induce sterilizing immunity against SARS-CoV-2 infections. One of these observations is that the RBD-specific antibodies were not only detected in serum but also in mucosal fluids (i.e., tear and nasal fluids) which are derived from the sites where the virus initially enters the body and infects host cells and initially replicates. A similar finding was obtained also for AIT vaccines which in fact block the docking of allergens to IgE antibodies bound to the effector cells of allergy at mucosal sites and thus prevent local allergic inflammation (Reisinger, J., et al., J Allergy Clin Immunol. 2005.116, 347-354; Shamji, M. H., et al., J Allergy Clin Immunol. 2019. 143, 1067-1076).

Another finding was that immunization with PreS-RBD induced not only a first short-lived wave of specific IgG₁ antibodies but also a second wave of late but sustained IgG₄ antibodies. In fact, it is known from AIT that AIT-induced allergen-specific IgG₄ antibodies persist in vaccinated subjects for a long time and are therefore considered to contribute to the long-term protective effect of AIT even after discontinuation of treatment (Larché, M., et al., Nat Rev Immunol. 2006. 6, 761-771; Shamji, M. H., et al. Allergy. 2021. 76, 3627-3641). Thus, PreS-RBD may have the potential to induce long-lasting sterilizing immunity against SARS-CoV-2 via induction of sustained production of RBD-specific IgG₄ antibodies which actually are considered as non-inflammatory neutralizing antibodies (van der Neut Kolfschoten, M., et al., Science. 2007. 317, 1554-1557).

There were no adverse events observed in the immunized rabbits of which each has received so far five doses of the vaccine. There were also no adverse side effects observed in the immunized subject.

In summary, the present example describes the in vitro and in vivo characterization of a SARS-CoV-2 subunit vaccine which has potential of inducing sterilizing immunity to SARS-CoV-2 variants.

### REFERENCES TO EXAMPLE 7

Argentinian AntiCovid Consortium. Structural and functional comparison of SARS-CoV-2-spike receptor binding domain produced in Pichia pastoris and mammalian cells. Sci Rep. 2020; 10(1):21779. Published 2020 Dec. 11. doi:10.1038/s41598-020-78711-6
Cornelius, C., et al. Immunotherapy With the PreS-based Grass Pollen Allergy Vaccine BM32 Induces Antibody Responses Protecting Against Hepatitis B Infection. EBioMedicine. 11, 58-67. doi:10.1016/j.ebiom.2016.07.023 (2016).
Eckl-Dorna, J., et al. Two years of treatment with the recombinant grass pollen allergy vaccine BM32 induces a continuously increasing allergen-specific IgG₄ response. EBioMedicine. 50, 421-432. doi:10.1016/j.ebiom.2019.11.006 (2019).
Gattinger, P., et al. Antibodies in serum of convalescent patients following mild COVID-19 do not always prevent virus-receptor binding. Allergy. 76, 878-883. doi:10.1111/all.14523 (2021).
Larché, M., Akdis, C. A., & Valenta, R. Immunological mechanisms of allergen-specific immunotherapy. Nat Rev Immunol. 6, 761-771. doi:10.1038/nri1934 (2006).
Paul, W. E., et al. Carrier function in anti-hapten immune responses. II. Specific properties of carrier cells capable of enhancing anti-hapten antibody responses. J Exp Med. 132, 283-299. doi:10.1084/jem.132.2.283 (1970).
Reisinger, J., et al. Allergen-specific nasal IgG antibodies induced by vaccination with genetically modified allergens are associated with reduced nasal allergen sensitivity. J Allergy Clin Immunol. 116, 347-354. doi:10.1016/j.jaci.2005.04.003 (2005).
Shamji, M. H., et al. Nasal allergen-neutralizing IgG₄ antibodies block IgE-mediated responses: Novel biomarker of subcutaneous grass pollen immunotherapy. J Allergy Clin Immunol. 143, 1067-1076. doi:10.1016/j.jaci.2018.09.039 (2019).
Shamji, M. H., et al. The role of allergen-specific IgE, IgG and IgA in allergic disease. Allergy. 76, 3627-3641. doi:10.1111/all.14908 (2021).
Tulaeva, I., et al. Quantification, epitope mapping and genotype cross-reactivity of hepatitis B preS-specific antibodies in subjects vaccinated with different dosage regimens of BM32. EBioMedicine. 59, 102953. doi:10.1016/j.ebiom.2020.102953 (2020).
Valenta, R., Campana, R., & Niederberger, V. Recombinant allergy vaccines based on allergen-derived B cell epitopes. Immunol Lett. 189, 19-26. doi:10.1016/j.imlet.2017.04.015 (2017).
van der Neut Kolfschoten, M., et al. Anti-inflammatory activity of human IgG₄ antibodies by dynamic Fab arm exchange. Science. 317, 1554-1557. doi:10.1126/science. 1144603 (2007).

1. An immunogenic subunit vaccine antigen which comprises at least two receptor-binding domains (RBDs) of the spike (S) protein of SARS-CoV-2 which are fused to a heterologous immunogenic carrier protein, wherein each of said at least two RBDs has a folded structure in an accessible conformation to bind human angiotensin-converting enzyme 2 (ACE2).

2. The vaccine antigen of claim 1, wherein at least one of said RBDs comprises or consists of an amino acid sequence of at least 180 amino acids length, and comprising at least 95% sequence identity to SEQ ID NO:1 or 2, optionally comprising one or more point mutations which are the same as comprised in an RBD of one or more different naturally-occurring SARS-CoV-2 mutants.

3. The vaccine antigen of claim 1, wherein said at least two RBDs consist of the same or different amino acid sequence.

4. The vaccine antigen of claim 1, wherein said folded structure is
a) obtained by expression of the vaccine antigen in a recombinant eukaryotic expression system, preferably employing mammalian, baculovirus-infected cells, or fungal host cells, preferably human host cells; and/or
b) determined by circular dichroism (CD) spectroscopy and/or an RBD-ACE2 interaction assay, preferably wherein the vaccine antigen is competing with a neutralizing anti-SARS-CoV-2 antibody preparation in the RBD-ACE2 interaction assay.

5. The vaccine antigen of claim 1, which is provided as a single chain fusion protein comprising said at least two RBDs fused to said heterologous immunogenic carrier protein, preferably comprising one or more peptide linker sequences.

6. The vaccine antigen of claim 1, wherein said vaccine antigen comprises two, three or more RBDs.

7. The vaccine antigen of claim 1, wherein the heterologous immunogenic carrier protein is a polypeptide that is not naturally fused to RBD.

8. The vaccine antigen of claim 1, wherein the heterologous immunogenic carrier protein is a viral protein such as a surface protein or nucleocapsid protein, or a protein domain of any of the foregoing.

9. The vaccine antigen of claim 1, wherein the heterologous immunogenic carrier protein is an antigen comprising B cell epitopes and T cell epitopes to elicit humoral and cellular immune responses in a human subject.

10. The vaccine antigen of claim 1, wherein the heterologous immunogenic carrier protein originates from any one of:
a) a virus of the Hepadnaviridae family, such as a human hepatitis virus or hepatitis B virus, preferably wherein the heterologous immunogenic carrier protein is a surface protein of hepatitis B virus, such as a PreS or S protein; or
b) a beta-coronavirus, preferably any one of SARS-CoV-2, SARS-CoV, MERS, HCoV-OC43 or HKU1, preferably wherein the heterologous immunogenic carrier protein is selected from the group consisting of the S protein, or a subdomain thereof, such as an S1 or S2 domain, or a nucleocapsid (N) protein; or
c) a human rhinovirus serotype, preferably wherein the heterologous immunogenic carrier protein is a viral capsid protein such as any one of VP1, VP2, VP3, or VP4; or
d) a RSV, preferably wherein the heterologous immunogenic carrier protein is a G-protein, or central conserved region of the G-protein; or
e) a glycolipid anchor, and wherein the RBDs fused to the anchor are surface expressed by a virus-like particle comprising a lipid bilayer envelope and a core protein of an enveloped virus, such as Moloney murine leukemia virus (MoMLV), preferably wherein the core protein is MoMLV Gag and/or Gag-Pol; or
f) a naturally-occurring mutant of any one of the foregoing.

11. The vaccine antigen of claim 1, wherein the heterologous immunogenic carrier protein is any other than an RBD of the spike (S) protein of SARS-CoV-2.

12. The vaccine antigen of claim 1, wherein the vaccine antigen comprises:
a) a single-chain fusion protein comprising at least two RBDs fused to a Hepatitis B Pres polypeptide of at least 50% length of any one of SEQ ID NO:19-26, and comprising at least 80% sequence identity to the corresponding region of the respective SEQ ID NO:19-26, preferably wherein at least one RBD is fused to the N-terminus and at least one RBD is fused to the C-terminus of the Pres polypeptide; and/or
b) at least two assembled RBDs which are each fused to a glycosyl phosphatidylinositol (GPI)-anchor and associated to the membrane of a virus-like particle expressed by a mammalian cell transfected with an expression plasmid encoding MoMLV gag-pol.

13. An isolated nucleic acid molecule encoding the vaccine antigen of claim 1, preferably comprising a polynucleotide sequence comprising at least 95% sequence identity to SEQ ID NO:17, or SEQ ID NO:18, or a codon-optimized variant of any of the foregoing, which is optimized to be expressed in a specific host cell line.

14. A vaccine comprising the vaccine antigen of claim 1 and any one or more of a pharmaceutically acceptable carrier, an excipient, or an adjuvant.

15. The vaccine of claim 14, wherein the adjuvant is selected from the group consisting of alum (aluminum phosphate gel or aluminum hydroxide gel or mixture of the two), AS04 (alum plus monophosphoryl lipid A), MF59 (oil-in-water emulsion adjuvant), and toll-like receptor agonist adjuvants (monophosphoryl lipid A plus CpG).

16. A method of
a) vaccinating a subject for prophylactic treatment against infection with SARS-CoV-2, including naturally-occurring mutants thereof, preferably to elicit neutralizing antibodies recognizing the natural RBD; and/or
b) treating a subject to induce antibodies against SARS-CoV-2, and/or to produce an antiserum or a blood plasma product which comprises antibodies against SARS-CoV-2, preferably wherein said antibodies are SARS-CoV-2 neutralizing antibodies,
the method comprising administering the vaccine of claim 14 to the subject.

17. The method of claim 16, wherein the vaccine is administered to the subject by subcutaneous, intramuscular, intranasal, microneedle, mucosal, skin, or transdermal administration.

18. A method for producing the vaccine antigen of claim 1, comprising expressing the vaccine antigen from an isolated nucleic acid molecule encoding the vaccine antigen in a recombinant eukaryotic expression system.

19. The method of claim 18, wherein the vaccine antigen is characterized by one or more of the following features:
a) the vaccine antigen comprises two, three or more RBDs;
b) said at least two RBDs consist of the same or different amino acid sequence;
c) at least one of said RBDs comprises or consists of an amino acid sequence of at least 180 amino acids length, and comprising at least 95% sequence identity to SEQ ID NO: 1 or 2, optionally comprising one or more point mutations which are the same as comprised in an RBD of one or more different naturally-occurring SARS-CoV-2 mutants;
d) said folded structure is
i. obtained by expression of the vaccine antigen in a recombinant eukaryotic expression system, preferably employing mammalian, baculovirus-infected cells, or fungal host cells, preferably human host cells; and/or
ii. determined by circular dichroism (CD) spectroscopy and/or an RBD-ACE2 interaction assay, preferably wherein the vaccine antigen is competing with a neutralizing anti-SARS-CoV-2 antibody preparation in the RBD-ACE2 interaction assay;
e) the vaccine antigen is provided as a single-chain fusion protein comprising said at least two RBDs fused to said heterologous immunogenic carrier protein, preferably comprising one or more peptide linker sequences;
f) the heterologous immunogenic carrier protein is a viral protein such as a surface protein or nucleocapsid protein, or a protein domain of any of the foregoing;
g) the heterologous immunogenic carrier protein is an antigen comprising B cell epitopes and T cell epitopes to elicit humoral and cellular immune responses in a human subject,
h) the heterologous immunogenic carrier protein is a polypeptide that is not naturally fused to RBD;
i. the heterologous immunogenic carrier protein originates from any one of:
i. a virus of the Hepadnaviridae family, such as a human hepatitis virus or hepatitis B virus, preferably wherein the heterologous immunogenic carrier protein is a surface protein of hepatitis B virus, such as a Pres or S protein; or
ii. a beta-coronavirus, preferably any one of SARS-CoV-2, SARS-CoV, MERS, HCoV-OC43 or HKU1, preferably wherein the heterologous immunogenic carrier protein is selected from the group consisting of the S protein, or a subdomain thereof, such as an S1 or S2 domain, or a nucleocapsid (N) protein; or
iii. a human rhinovirus serotype, preferably wherein the heterologous immunogenic carrier protein is a viral capsid protein such as any one of VP1, VP2, VP3, or VP4; or
iv. a RSV, preferably wherein the heterologous immunogenic carrier protein is a G-protein, or central conserved region of the G-protein; or
v. a glycolipid anchor, and wherein the RBDs fused to the anchor are surface-expressed by a virus-like particle comprising a lipid bilayer envelope and a core protein of an enveloped virus, such as Moloney murine leukemia virus (MoMLV), preferably wherein the core protein is MoMLV Gag and/or Gag-Pol; or
vi. a naturally-occurring mutant of any one of the foregoing;
j) the heterologous immunogenic carrier protein is any other than an RBD of the spike (S) protein of SARS-CoV-2;
k) the heterologous immunogenic carrier protein is any one of:
i. a Hepatitis B Pres polypeptide of at least 50% length of any one of SEQ ID NO: 19-26, and comprising at least 80% sequence identity to the corresponding region of the respective SEQ ID NO:19-26, preferably wherein at least one RBD is fused to the N-terminus and at least one peptide is fused to the C-terminus of the Pres polypeptide; and/or
ii. a glycosyl phosphatidylinositol (GPI)-anchor which is associated to the membrane of a virus-like particle expressed by a mammalian cell transfected with an expression plasmid encoding MoMLV gag-pol.

20. A method for producing a vaccine by formulating the vaccine antigen of claim 1 with any one or more of a pharmaceutically acceptable carrier, an excipient, or an adjuvant.

21. A method of producing an RBD subunit vaccine with increased immunogenicity by fusing at least a first and a second folded RBDs to said heterologous immunogenic carrier protein.

22. The method of claim 21, wherein the heterologous immunogenic carrier protein is any one of:
a) a Hepatitis B PreS polypeptide of at least 50% length of any one of SEQ ID NO: 19-26, and comprising at least 80% sequence identity to the corresponding region of the respective SEQ ID NO: 19-26, preferably wherein at least one RBD is fused to the N-terminus and at least one peptide is fused to the C-terminus of the Pres polypeptide; and/or
b) a glycosyl phosphatidylinositol (GPI)-anchor which is associated to the membrane of a virus-like particle expressed by a mammalian cell transfected with an expression plasmid encoding MoMLV gag-pol.
